SEQUENCE LISTING (4 sequences)

SEQ ID NO: 1 - DNA, 20 bp, Artificial Sequence
  synthetic construct
gaccccaaaatcagcgaaat

SEQ ID NO: 2 - DNA, 24 bp, Artificial Sequence
  synthetic construct
tctggttactgccagttgaatctg

SEQ ID NO: 3 - DNA, 17 bp, Artificial Sequence
  synthetic construct
ccacctttgacgctggg

SEQ ID NO: 4 - DNA, 23 bp, Artificial Sequence
  synthetic construct
cataccaggaaatgagcttgaca

(19) United States
(12) Patent Application Publication
(10) Pub. No.: US 2023/0174946 A1
(43) Pub. Date: Jun. 8, 2023

(54) ORGAN INFECTION MODELS

(71) Applicants: CEDARS-SINAI MEDICAL CENTER, Los Angeles, CA (US); THE REGENTS OF THE UNIVERSITY OF CALIFORNIA, Oakland, CA (US)

(72) Inventors: Barry R. Stripp, Malibu, CA (US); Apoorva Mulay, Los Angeles, CA (US); Bindu Konda, Redondo Beach, CA (US); Arun Sharma, Los Angeles, CA (US); Clive Svendsen, Pacific Palisades, CA (US); Vaithilingaraja Arumugaswami, Los Angeles, CA (US); Dhruv Sareen, Porter Ranch, CA (US); Hanan Shaharuddin, Sun Valley, CA (US); Victoria Wang, San Diego, CA (US); Roberta S. Santos, Los Angeles, CA (US)

(73) Assignees: CEDARS-SINAI MEDICAL CENTER, Los Angeles, CA (US); THE REGENTS OF THE UNIVERSITY OF CALIFORNIA, Oakland, CA (US)

(21) Appl. No.: 17/922,162

(22) PCT Filed: Apr. 30, 2021
(86) PCT No.: PCT/US2021/030322

§ 371 Date: Oct. 28, 2022

Related U.S. Application Data
(60) Provisional application No. 63/019,217, filed on May 1, 2020.
(60) Provisional application No. 63/054,440, filed on Jul. 21, 2020.
(60) Provisional application No. 63/130,158, filed on Dec. 23, 2020.

(51) Int. Cl. C12N 5/071; C12N 5/077; C12M 3/06

(52) U.S. Cl. CPC C12N 5/0676; C12M 23/16; C12N 5/0657; C12N 5/0688; C12N 2503/02; C12N 2506/45

(57) ABSTRACT

Described herein are particular infection model systems, methods of studying infection, and method of screening compounds in various model systems. Particularly, SARS-CoV-2 is studied in these organ and infection models.

### CROSS-REFERENCE TO RELATED APPLICATIONS

This application includes a claim of priority under 35 U.S.C. §119(e) to U.S. Provisional Pat. Applications No. 63/019,217, filed May 1, 2020, No. 63/054,440, filed Jul. 21, 2020, No. 63/130,158 filed Dec. 23, 2020, the entirety of all of which are hereby incorporated by reference.

### STATEMENT REGARDING FEDERALLY SPONSORED RESEARCH OR DEVELOPMENT

This invention was made with government support under Grant Nos. NS105703 and HL108793 awarded by National Institutes of Health. The government has certain rights in the invention.

### FIELD OF INVENTION

This invention relates to infection models and drug screening.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Air spaces of the human respiratory system can be broadly divided into conducting and respiratory zones that mediate transport of gasses and their subsequent exchange across the epithelial-microvascular barrier, respectively. The conducting airways include the trachea, bronchi, bronchioles and terminal bronchioles whereas respiratory air spaces include the respiratory bronchioles, alveolar ducts and alveoli. The epithelial lining of these airspaces changes in composition along the proximo-distal axis to accommodate the unique requirements of each functionally distinct zone. The pseudostratified epithelium of tracheo-bronchial airways is composed of three major cell types, basal, secretory and ciliated, in addition to less abundant cell types including brush, neuroendocrine and ionocyte. Bronchiolar airways harbor morphologically similar epithelial cell types, although there are distinctions in their abundance and functional properties. For example, basal cells are less abundant within bronchiolar airways and secretory cells include a greater proportion of club cells versus serous and goblet cells that predominate in tracheobronchial airways. Epithelial cells of the respiratory zone include a poorly defined cuboidal cell type in respiratory bronchioles, in addition to alveolar type I (ATI) and type II (ATII) cells of alveolar ducts and alveoli.

The identity of epithelial stem and progenitor cell types that contribute to maintenance and renewal of epithelia in each zone are incompletely described and largely inferred from studies in animal models. Studies in mice have shown that either basal cells of pseudostratified airways, club cells of bronchiolar airways or ATII cells of the alveolar epithelium, serve as epithelial stem cells based upon capacity for unlimited self-renewal and multipotent differentiation. Despite the inability to perform genetic lineage tracing studies to assess stemness of human lung epithelial cell types, the availability of organoid-based culture models to assess the functional potential of epithelial stem and progenitor cells provides a tool for comparative studies between mouse and human. Herein the Inventors describe methods for isolation and functional analysis of epithelial cell types from different regions of the human lung.

Described herein is tissue dissociation and cellular fractionation approaches allowing enrichment of epithelial cells from proximal and distal regions of the human lung. Herein these approaches are applied to the functional analysis of lung epithelial progenitor cells through use of 3D organoid culture models.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a system for infection modeling or test agent screening, comprising: a population of cells comprising cells selected from the group consisting of progenitor cells derived from induced pluripotent stem cells (iPSCs), cells differentiated from iPSCs, an organoid comprising the cells differentiated from iPSCs, or primary cells; and a fluidic device, a cell culture plate, or a multi-well culture plate; wherein an infectious agent and the population of cells, or the infectious agent, a test agent, and the population of cells, are in contact in the fluidic device, the cell culture plate, or the multi-well culture plate.

In various embodiments, the fluidic device can be an air-liquid interface culture or a Transwell system comprising the population of cells. In various embodiments, the fluidic device can be a microfluidic device comprising the population of cells. In various embodiments, the microfluidic device can be an organ chip.

In various embodiments, when the fluidic device is an air-liquid interface culture, a Transwell system, a microfluidic device or an organ chip, the progenitor cells derived from induced pluripotent stem cells (iPSCs) can comprise progenitor heart cells or progenitor endothelial cells. In various embodiments, the primary cells can comprise heart cells or endothelial cells. In various embodiments, the heart cells can be cardiac myocytes.

In various embodiments, when the fluidic device is an air-liquid interface culture, a Transwell system, or a microfluidic device, the progenitor cells derived from induced pluripotent stem cells (iPSCs) can comprise progenitor lung cells or progenitor pancreatic cells. In various embodiments, the primary cells can comprise lung cells or pancreatic cells. In various embodiments, the lung cells can be epithelial cells. In various embodiments, the epithelial cells can be proximal airway cells, or distal alveolar cells.

In various embodiments, the infectious agent can be a virus, a bacterium, a fungus, or a parasite. In various embodiments, the virus can be a coronavirus. In various embodiments, the coronavirus is SARS-CoV-1, MERS, or SARS CoV-2.

In various embodiments, the test agent can be an anti-viral agent. In various embodiments, the test agent can be a nucleotide analog, an anti-inflammatory agent, interferon beta, ribavirin, chloroquine, azithromycin, favipiravir, lopinavir/ritonavir or remdesivir.

Various embodiments provide for a method selecting an agent of interest, comprising: contacting a test agent with a population of cells comprising cells selected from the group consisting of progenitor cells derived from induced pluripotent stem cells (iPSCs), cells differentiated from iPSCs, an organoid comprising the cells differentiated from iPSCs, or primary cells, wherein the test agent and the population of cells are in contact in a fluidic device, a cell culture plate, or a multi-well culture plate; infecting the population of cells with an infectious agent before, simultaneously or after contacting the test agent with the population of cells; measuring a parameter in the population of cells; and selecting the test agent as the agent of interest based on the measured parameter in the population of cells.

Various embodiments provide for a method of studying an infectious agent, comprising: contacting an infectious agent with a population of cells comprising cells selected from the group consisting of progenitor cells derived from induced pluripotent stem cells (iPSCs), cells differentiated from iPSCs, an organoid comprising the cells differentiated from iPSCs, or primary cells, wherein the infectious agent and the population of cells are in contact in a fluidic device, a cell culture plate, or a multi-well culture plate; and measuring a parameter in the population of cells.

In various embodiments, the parameter can comprise a phenotype of interest, an expression level of a gene, or an expression level of a protein of interest, or combination thereof

In various embodiments, the fluidic device can be an air-liquid interface culture or a Transwell system comprising the population of cells.

In various embodiments, the fluidic device can be a microfluidic device comprising the population of cells.

In various embodiments, progenitor cells derived from induced pluripotent stem cells (iPSCs) can comprise progenitor lung cells, progenitor heart cells, progenitor endothelial cells, or pancreatic progenitor cells.

In various embodiments, the primary cells can comprise lung cells, heart cells, endothelial cells, or pancreatic cells. In various embodiments, the lung cells are epithelial cells. In various embodiments, the epithelial cells can be proximal airway cells, or distal alveolar cells.

In various embodiments, the heart cells can be cardiac myocytes.

In various embodiments, the infectious agent can be a virus, a bacterium, a fungus, or a parasite. In various embodiments, the virus can be a coronavirus. In various embodiments, the coronavirus can be SARS-CoV-1, MERS or SARS CoV-2.

In various embodiments, the test agent can be an anti-viral agent. In various embodiments, the test agent can be a nucleotide analog, an anti-inflammatory agent, interferon beta, ribavirin, chloroquine, azithromycin, favipiravir, lopinavir/ritonavir or remdesivir.

In various embodiments, the parameter can comprise a phenotype of interest, expression level of a gene of interest, expression level of a protein of interest, or combinations thereof in the population of cells.

Various embodiments of the present invention provide for a method of selecting an agent of interest, comprising: contacting a test agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; measuring a phenotype of interest or expression level of a gene or protein of interest in the population of cells; selecting the test agent as the agent of interest based on the measurement of the phenotype of interest or expression level of the gene or protein of interest.

Various embodiments of the present invention provide for a method of selecting an agent of interest, further comprising: infecting the population of cells described herein, with an infectious agent before, simultaneously or after contacting the test agent with the population of cells.

Various embodiments of the present invention provide for a method of studying an infectious agent, comprising: contacting an infectious agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; and measuring a phenotype of interest or expression level of a gene or protein of interest in the population of cells.

In various embodiments, the test agent and the population of cells can be in contact in a fluidic device, or a cell culture plate, or a multi-well culture plate. In various embodiments, the fluidic device, or the cell culture plate, or the multi-well culture plate can be a Transwell system. In various embodiments, the fluidic device can be a microfluidic device.

In various embodiments, the population of cells can be selected from the group consisting of pancreatic progenitor cells derived from induced pluripotent stem cells (iPSCs), pancreatic ductal cells derived from iPSCs, pancreatic acinar cells derived from iPSCs, an organoid comprising derived from iPSCs, an organoid comprising pancreatic acinar cells derived from iPSCs, an organoid comprising pancreatic ductal cells derived from iPSCs, and combinations thereof.

In various embodiments, the population of cells can be selected from the group consisting of pancreatic ductal cells derived from pancreatic progenitor cells, pancreatic acinar cells derived from pancreatic progenitor cells, an organoid comprising pancreatic acinar cells derived from pancreatic progenitor cells, an organoid comprising pancreatic ductal cells derived from pancreatic progenitor cells, and combinations thereof.

In various embodiments, the infectious agent can be a virus, bacterium, parasite, or fungus. In various embodiments, the infectious agent can be a coronavirus. In various embodiments, the infectious agent can be SARS-CoV-2.

In various embodiments, the test agent can be selected from the group consisting of interferon beta, ribavirin, chloroquine, azithromycin, favipiravir, lopinavir/ritonavir, and remdesivir.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels A-D) depicts isolation strategy (A) Representative image of the human lung showing proximal and distal regions selected for cell isolation. (B) H&E staining of the proximal and distal regions of the lung. Proximal airways show a pseudostratified epithelium whereas the distal regions show presence of alveoli. (C, D) Immunofluorescent staining of corresponding regions showing NGFR⁺ basal progenitor cells at the basement membrane of the proximal airways and HTII-280⁺ alveolar type II progenitors in the distal airways. Scale bare is 50 µm.

FIG. 2 (panels A-H) depicts sorting strategy for distal lung (A & E) Percentage of various cell populations before and after depletion of CD45⁺and CD31⁺ population using CD31 and CD45 magnetic beads in distal regions of the lung. (B & F) Representative image of FACS plot showing gating strategy of distal CD31⁻/CD45⁻/CD235a⁻ population before and after depletion of CD31/CD45/CD235a positive population (C & F) Epcam⁺ population before and after depletion of CD31/CD45/CD235a positive population. (D & H) HT II-280⁺/⁻ population before and after depletion of CD31/CD45/CD235a positive cells. FIGS. 2A, 2B, 2C and 2D are from the same biological sample and FIGS. 2E, 2F, 2G and 2H are from the same biological sample.

FIG. 3 (panels A-H) depicts sorting strategy for proximal lung (A & E) Percentage of various cell populations before and after depletion of CD45+and CD31+ population using CD31 and CD45 magnetic beads in proximal regions of the lung. (B & F) Representative image of FACS plot showing gating strategy of proximal CD31⁻/CD45⁻/CD235a⁻ population before and after depletion of CD31/CD45/CD235a positive population (C & F) Epcam⁺ population before and after depletion of CD31/CD45/CD235a positive population. (D & H) NGFR⁺/⁻ population before and after depletion of CD31/CD45/CD235a positive cells. FIGS. 3A, 3B, 3C and 3D are from the same biological sample and FIGS. 3E, 3F, 3G and 3H are from the same biological sample.

FIG. 4 (panels A-D, C′ D′) depicts characterization of distal organoids (A, B) Representative image of the human distal organoids cultured in PneumaCult™-ALI medium (2X) and (10X). (B) The Colony forming efficiency (%CFE) was calculated on triplicate wells of organoids derived from two biological samples. (C, D) Immunofluorescent staining of corresponding distal organoid cultures in ALI medium showing HT II-280⁺ AT2 cells (green) and nuclei (blue). (C′, D′) higher magnification of Immunofluorescent staining of corresponding distal organoids cultured in PneumaCult™-ALI medium showing HTII-280+AT2 cells (green) and nuclei (scale bar= 50 mm).

FIG. 5 (panels A-F) depicts characterization of Proximal organoids from the human Proximal lung (A, B) Representative image of the human Proximal organoids cultured in PneumaCult™-ALI medium scale bar 50 mm. (C) The Colony forming efficiency (%CFE) was calculated on triplicate wells of organoids derived from three biological replicates. (D) Immunofluorescent staining of differentiated proximal organoids expressing Acetylated tubulin (red). (E) FOXJ1 (red) marking ciliated cells (F) goblet cells expressing MUC5AC (red). (E, F) basal cells expressing K5 (green) at day 30. scale bar = 50 mm.Table 1: Cell isolation

FIG. 6 (panels A-I) shows comparing sorting strategy for mouse lung (Fresh Tissue VS Frozen Tissue Vs frozen Lung cells): A, B & C are from same biological sample (Fresh mouse lung). (A) Flow cytometry of mouse fresh lung cells suspension showing gating strategy. (B) Representative image of FACS plot showing gating strategy of CD31-/CD45-population and (C) Epcam+ population. D, E & F are from same biological sample (Frozen mouse lung tissue). (D) Flow cytometry of cells isolated from mouse frozen lung tissue, showing gating strategy. (E) FACS plot showing gating strategy of CD31-/CD45-population and (F) Epcam+ population. G, H & I are from same biological sample (frozen mouse lung cells). (G) Flow cytometry of frozen mouse lung cells showing gating strategy. (H) FACS plot showing gating strategy of CD31-/CD45 population and (I) Epcam+ population.

FIG. 7 (Panels A-L) shows comparing sorting strategy for human distal lung (Fresh Tissue VS Frozen Tissue Vs Frozen Lung cells): A to L are from same biological sample with different conditions (Fresh Tissue, Frozen Tissue and Frozen cells) and sorted on same day. (A) Aating strategy for cells isolated from fresh distal lung tissue, (B) percentage of live cells by DAPI staining, (C) gating strategy for CD31⁻/CD45⁻ population, (D) Epcam⁺ and HT II- 280⁺ population. (E) showing gating strategy for cells isolated from frozen distal lung tissue, (F) percentage of live cells by DAPI staining, (G) gating strategy for CD31⁻/CD45⁻ population and (H) Epcam⁺ and HT II-280⁺ population. (I) showing gating strategy for frozen distal lung cells, (J) percentage of live cells by DAPI staining, (k) gating strategy for CD31⁻/CD45⁻ population and (L) Epcam⁺ and HT II- 280⁺ population.

FIG. 8 (panels A-G) depict Colony Forming Efficiency of HT II-280⁺ cells from Human Distal lung. Fresh Lung Tissue Vs Frozen Lung Tissue Vs Frozen Cells. A, B, C & D are from same biological sample. (A) Representative images of organoids formed from HT II-280⁺ cells, day 20 from Fresh distal lung, (B) Frozen Distal tissue and (C) Frozen distal lung cells. 2000 cells / well were added and cultured in SAEGM medium. (D) Colony Forming Efficiency of HT II-280⁺ cells from the corresponding sample measured on day 20. Representative images of organoids formed from HT II-280⁺ cells from two different biological distal lung samples. 5000 cells/well were cultured in PneumaCult™ ALI medium for 30 days. (E) Organoids culture from Fresh distal lung tissue, (F) Organoids cultured from Frozen Tissue. (G) Colony Forming Efficiency of HT II-280+ cells on day 30 from two biological samples with triplicates.

FIG. 9 (panels A-L) shows Comparing Sorting Strategy for Human Proximal Lung (Fresh VS Frozen Vs Frozen Lung cells): A to L are from same biological sample with different conditions (Fresh Tissue, Frozen Tissue and Frozen cells) and sorted on same day. (A) Gating strategy for cells isolated from fresh proximal lung tissue, (B) gating strategy for CD31⁻/CD45⁻ population, (C) Epcam⁺ and (D) NGFR⁺ population. (E) Showing gating strategy for cells isolated from frozen proximal lung tissue (F) gating strategy for CD31⁻/CD45⁻ population, (G) Epcam⁺ and (H) NGFR⁺ population. (I) Showing gating strategy for proximal frozen lung cells, (J) gating strategy for CD31⁻/CD45⁻ population, (K) Epcam⁺ and (L) NGFR⁺ population.

FIG. 10 (panels A-G) depicts Colony Forming Efficiency of NGFR⁺ cells from Human Proximal lung. Fresh Lung Tissue Vs Frozen Lung Tissue Vs Frozen Cells. A, B, C & D are from same biological sample. (A) Representative images of organoids formed from NGFR⁺ cells, day 20 from Fresh proximal lung, (B) Frozen proximal tissue and (C) Frozen proximal lung cells. 2000 cells / well were added and cultured in PneumaCult EX Basal medium. (D) Colony Forming Efficiency of Epcam⁺ cells from the corresponding sample measured on day 20. (E, F & G) Representative images of organoids formed from NGFR⁺ cells from two different biological proximal lung samples. 5000 cells/well were cultured in PneumaCult™ ALI medium for 30 days. (E) Organoids culture from Fresh proximal lung tissue, (F) Organoids cultured from Frozen Tissue. (G) Colony Forming Efficiency of NGFR⁺ cells on day 30 from two biological samples with triplicates.

FIG. 11 (panels A-G) shows Comparing Colony Forming Efficiency and Sorting profile in Fresh tissue vs Frozen tissue for both Distal Lung and Proximal Lung. A to H are from same biological sample. (A & B) Organoids formed from HT II-280⁺ cell and FACS profile of Human Fresh Distal tissue. (C & D) Organoids formed from HT II-280⁺ cell and FACS profile of Frozen Distal tissue. (E & F) organoids formed from NGFR⁺ cell and FACS profile of Fresh Proximal tissue. (G & H) Organoids formed from NGFR⁺ cell and FACS profile of Frozen Proximal tissue (A & C) Colony Forming Efficiency is similar in both fresh and frozen distal tissue and (E & G) proximal fresh and frozen tissue.

FIG. 12 (panels A-B) depicts Characterization and Comparing Immunohistochemistry of Distal Organoids from Fresh Tissue Vs Frozen Tissue. (A) Representative immunofluorescent staining of human fresh distal organoids cultured in PneumaCultTM -ALI and (B) frozen distal organoids showing HT II-280⁺ AT2 cells (green) and nuclei (blue). Scale bar = 50 mm.

FIG. 13 (panels A-C) depicts Characterization and Comparing Immunohistochemistry of Proximal Organoids from Fresh Tissue Vs Frozen Tissue. (A) Immunofluorescent staining of differentiated proximal organoids expressing MUC5AC (red) in goblet cells and basal cells expressing K5 (green) at day 30 from fresh Proximal lung tissue and (B) Frozen proximal lung tissue. (C) FOXJ1 (red) marking ciliated cells and basal cells expressing K5 (green) at day 30 from fresh proximal tissue. Scale bar= 50 mm.

FIG. 14 shows an example of lung cells infected with SARS-CoV-2 in the presence of candidate drug agents.

FIG. 15 shows an example of lung cells infected with SARS-CoV-2 in the presence of candidate drug agents.

FIG. 16 shows an example of lung cells infected with SARS-CoV-2 in the presence of candidate drug agents.

FIG. 17 shows a Flowchart of research strategy. Expression of ACE2 gene in hiPSCs and hiPSC-CMs.

FIG. 18 (panels A-G) depicts SARS-CoV-2 is internalized and replicates within hiPSC-CMs in vitro, eliciting cytopathic effect and contractility alterations. A) Human iPSC-CMs exhibit standard sarcomeric markers including cardiac troponin T (cTnT) and alpha-actinin. DAPI stains DNA in cell nuclei. B) HiPSC-CMs grown in monolayer can be infected by SARS-CoV-2, as indicated by immunofluorescence for cTnT and SARS-CoV-2 “spike” protein. Cells were subjected to SARS-CoV-2 at multiplicity of infection (MOI) of 0.1 for 3 days before fixing in paraformaldehyde for immunofluorescence. C) After SARS-CoV-2 infection, hiPSC-CMs exhibit signs of cellular apoptosis, indicated by cleaved caspase-3 expression and morphological changes seen under bright field (BF). A second SARS-CoV-2 antibody marks a viral-specific double-stranded intermediate RNA (dsRNA). Cells were subjected to SARS-CoV-2 at multiplicity of infection (MOI) of 0.1 for 3 days before fixing in paraformaldehyde for immunofluorescence. D) Magnified inset from panel B showing a merged immunofluorescence image for SARS-CoV-2 spike protein and DAPI. Arrows indicate perinuclear accumulation of viral particles, and suggests active viral protein translation and genome replication at perinuclear ribosomes and membranous compartments. E) Magnified inset from panel C showing a merged immunofluorescence image for SARS-CoV-2 dsRNA and DAPI. Arrows indicate perinuclear viral replication sites. F) Quantification of immunofluorescence indicating percentage of cells positive for spike protein, viral dsRNA, cleaved caspase-3 (CC3), and dsRNA+CC3. N=5-7 images quantified for each stain for mock and infected conditions. * indicates p < 0.05. G) Quantification of beats per minute in wells containing mock control hiPSC-CMs versus wells containing hiPSC-CMs infected with SARS-CoV-2 for 72 hours. N=6 for each condition.

FIG. 19 (panels A-D) shows that SARS-CoV-2 infects and replicates within normal human proximal airway cells. (A) Workflow for establishment of human proximal airway ALI cultures and their infection with SARS-CoV-2. (B) ALI cultures of proximal airway epithelial cells are susceptible to SARS-CoV-2 infection which peaked at 2 dpi; n= 3 to 6 cultures from 2 independent donors. Circles indicate mock cultures and triangles indicate SARS-CoV-2 infected cultures. Red and green colors indicate cultures from separate donors. Data were analyzed using Two-Way ANOVA with Sidak’s post-hoc correction and represented as fold change for individual cultures ± SEM relative to mean N gene expression at 1 dpi ****p<0.0001. 2 days after SARS-CoV-2 infection, viral spike protein (green) was found to heterogeneously co-localize with (C) acetylated tubulin positive ciliated cells (red) and (D) a proportion of Muc5AC positive goblet cells (red). Scale bar = 20 µm. Arrows indicate colocalization of markers.

FIG. 20 (panels A-E) shows that SARS-CoV-2 infects and replicates within normal human distal alveolar organoids. (A) Workflow for establishment of human distal alveolar organoid cultures and their infection with SARS-CoV-2. (B) Enzymatic and mechanical disruption of alveolar organoids to expose the apical surface was essential for robust infection, as evaluated by detection of SARS-CoV-2 N gene abundance; n = 3 to 4 organoid cultures established from 2 different donor samples for each condition. (C) Viral infection levels peaked at 2 days post infection; n=3 organoid cultures. Data were analyzed using Two-Way ANOVA with Sidak’s post- hoc correction and represented as fold change for individual cultures ± SEM relative to mean N gene expression at 1 dpi. ****p=0.0002 for (B) and ***p=0.0009 ****p<0.0001 for (C). (D) Viral infection was assessed at 2 dpi by antibody against SARS-CoV-2 “spike” protein (green) and colocalized with AT2 cell marker HTII-280 (red) (E) Infected alveolar organoids (red) also demonstrated signs of cellular apoptosis at 3 dpi, indicated by positive staining for cleaved caspase-3 (green); scale bar= 20 µm.

FIG. 21 (panels A-F) shows Primary human lung epithelial models for study of SARS-CoV-2 induced host and drug validation. (A) Heatmap showing RNA seq analysis of differentially expressed genes at 2 dpi. (B) Volcano plot of gene expression changes in SARS-CoV-2 infected vs mock cultures defined by p-value and >2-fold change. Several viral genes such as Virus_N, virus_ORFlab, virus_ORF3a were detected in the infected samples. Cytokines such as IFNB1, and antiviral response genes OAS1, GAS2, ISG15 and MX1 were significantly upregulated. (C) The most upregulated canonical transcriptional pathway in SARS-CoV-2 infected alveolar cultures was IFN signaling pathway. In addition to TLR and NFKB signaling. (D) Downregulated canonical transcriptional pathways include antigen presentation and Th1 and Th2 activation pathways. (E) Pre-treatment of alveolar organoid cultures with IFNB1, hydroxychloroquine and Remdesivir significantly reduced viral replication. The effect of Remdesivir on viral replication was more pronounced than that of IFNB1 or hydroxychloroquine. Data are represented as log2fold change for individual cultures ± SEM, normalized to mean infection and analyzed using One-Way ANOVA with Tukey’s post-hoc test **p=0.0012 for IFNB1, **p=0.0044 for HCQ and ****p<0.0001 for Remdesivir. The 3 different colors indicate cultures from different biological replicates. (F) Pre-treatment of proximal ALI cultures with Remdesivir significantly reduced viral infection/replication. Pre-treatment with hydroxychloroquine and IFNB did not have an effect on viral replication; n= 3-4 independent cultures. Data are represented as log2fold change for individual cultures, normalized to mean infection and analyzed using One-Way ANOVA plus Tukey’s posthoc test ****p<0.0001.

FIG. 22 shows that SARS-CoV-2 induces cell-autonomous and non-cell-autonomous apoptosis of AT2 cells. Number of cleaved-caspase 3 positive cells was significantly higher in SARS-CoV-2 infected alveolar organoids compared to mock alveolar organoids. Only a percentage of the apoptotic cells were positive for viral spike protein indicating that SARS-Co-V2 infections induces apoptosis of neighboring cells in addition to infected cells. Spike protein staining was indetectable in mock cultures. N= 7 different images of alveolar organoids for each condition. Data are represented as mean percentage of cells staining positive for target protein ± SEM and analyzed using Two-Way ANOVA plus Sidak’s post-hoc test ****p<0.0001.

FIG. 23 shows the Effect of drugs on 3D alveolar organoid cultures from individual biological replicates. Effect of treatment of IFNB1, hydroxychloroquine and remdesivir on 3D organoid cultures from 3 different donors showed variability for IFNB1 and hydroxychloroquine effect amongst donors; Variability among donors for hydroxychloroquine effect was higher. However, Remdesivir showed consistently strong inhibition of viral replication/infection irrespective of donor origin of cells. N= 2 to 3 cultures from 3 independent biological replicates. Data are represented as log2foldchange for individual cultures ± SEM, normalized to mean infection for individual biological replicates and analyzed using One-Way ANOVA plus Tukey’s post-hoc test within individual biological replicate Donor 1: *p=0.021 ****p<0.0001; Donor 3: ** p=0.0024 ****p<0.0001.

FIG. 24 (panels A-G) shows that SARS-CoV-2 infects and replicates within 1 normal human proximal airway cells. (A) Workflow for establishment of human proximal airway ALI cultures and their infection with SARS-CoV-2. (B) ALI cultures of proximal airway epithelial cells are susceptible to SARS-CoV-2 infection which peaked at 2 dpi; n= 3 to 6 cultures from 2 independent donors. Circles indicate mock cultures and triangles indicate SARS-CoV-2 infected cultures. Red and green colors indicate cultures from separate donors. Data were analyzed using Two-Way ANOVA with Sidak’s posthoc correction and represented as fold change for individual cultures ± SEM relative to mean N gene expression at 1 dpi ****p<0.0001. 2 days after SARS-CoV-2 infection, viral spike protein (green) was found to heterogeneously co-localize with (C) Percentage of cells from proximal airway ALI cultures infected with SARS-CoV-2 at 2 dpi. n= 14-15 fields from 2 biological replicates. (D) FOXJ1 positive ciliated cells (red) and (E) MUC5AC positive goblet cells (red) infected by SARS-CoV-2 (green). Scale bar = 20 µm. (F) Percentage of infected cells that are either ciliated cells or goblet cells. Change in the percentage of (G) ciliated and (H) goblet cells post SARS-CoV-2 infection. n= 7-8 fields from 2 biological replicate. Data is presented as mean± SEM (significance determined by two tailed t-test). *p<0.05.

FIG. 25 (panels A-L) shows SARS-CoV-2 infects and replicates within normal human distal AT2 cells. (A) Sections of Matrigel® embedded alveospheres showing colocalization of AT2 markers HTII 280 and SPC. (B) Whole mount staining of alveospheres dispersed from Matrigel® for AT2 marker HTII 280. (C) Percentage of HTII 280 positive and HTII 280 negative cells in alveospheres n=3-5 fields from 3 biological replicates (Two tailed t test) (D) Whole mount alveospheres stained for the AT2 marker, HTII 280 and AT1 cell marker HTI 56. ACE2 staining in sectioned alveospheres (E) and in AT2 cells dissociated from alveospehers. (G) Workflow for establishment of human distal AT2 cultures and their infection with SARS-CoV-2. (H) N gene expression in SARS-CoV-2 infected cultures peaked at 2 days post infection; n=3 independent cultures. Data were analyzed using Two-Way ANOVA with Sidak’s post-hoc correction and represented as fold change for individual cultures ± SEM relative to mean N gene expression at 1 dpi. (I) Viral load in supernatant increased from 1 dpi to 3 dpi ***p<0.001 ****p<0.0001. (J) Percentage of cells infected (n=3) ***p<0.001. (K) Viral infection was assessed at 2 dpi by antibody against SARS-CoV-2 “spike” protein (green) and colocalized with AT2 cell marker HTII-280 (red). (L) Percentage of HTII 280 positive cells infected by SARS-CoV-2; n=3-5 fields from each of 3 biological replicates.

FIG. 26 (panels A-G) shows Primary human AT2 cultures as a model to study SARS-CoV-2 induced host response. (A) Principal component analysis of infected AT2 cultures and MOCK cultures showing variance in the global transcriptome of the two groups (n= 5 from 3 biological replicates for both MOCK and SARS-CoV-2 infected cultures). (B) Heatmap showing RNA seq analysis of differentially expressed genes between SARS-CoV-2 infected and MOCK AT2 cultures at 2 dpi. (C) Volcano plot of gene expression changes in SARS-CoV-2 infected vs mock cultures defined by p-value and >2-fold change. (D) Heat map of normalized TPM counts for various Interferon ligands, receptors and ISGs. (E) Quantification of normalized TPM counts of various downstream ISGs between MOCK and SARS-CoV-2 infected cultures (n=5; significance determined by two-tailed t-test) *p<0.05 **p<0.01 ***p<0.001 ****p<0.0001. The most highly upregulated (F) and downregulated (G) canonical pathways in SARS-CoV-2 infected alveolar cultures.

FIG. 27 shows SARS-1 CoV-2 infection triggers apoptosis of alveolar cells. (A) Quantification of normalized TPM counts for apoptosis related genes between MOCK and SARS-CoV-2 infected AT2 cultures (n=5; significance determined by two-tailed t-test). *p<0.05 **p<0.01 ***p<0.001 ****p<0.0001 (B) Infected AT2 cells (red) also demonstrated signs of cellular apoptosis at 3 dpi, indicated by positive staining for cleaved caspase-3 (green) scale bar= 20 µm. (C) Comparison of percentage of cells staining positive for CC3 in intact Matrigel® embedded MOCK alveospheres, AT2 cultures dispersed from Matrigel® and infected cultures dispersed from Matrigel® n=7 (One Way ANOVA with Tukey’s posthoc test). *p<0.05; **p<0.01; ***p<0.001, ****p<0.0001. (D) Pre-treatment of AT2 cultures with IFNB1, Hydroxychloroquine and Remdesivir significantly reduced viral replication. The effect of Remdesivir on viral replication was more pronounced than that of IFNB1 or hydroxychloroquine. Data are represented as log2fold change for individual cultures ± SEM, normalized to mean infection and analyzed using One-Way ANOVA with Tukey’s post-hoc test **p=0.0012 for IFNB1, **p=0.0044 for HCQ and ****p<0.0001 for Remdesivir. The 3 different colors indicate cultures from different biological replicates.

FIG. 28 depicts SARS-CoV-2 induces an interferon 1 proinflammatory response in AT2 cells. Quantification of TPM counts for Type 1 and Type 3 interferon ligands (A) and various interferon receptors (B) for n=5 independent cultures from 3 biological replicates. Data is presented as mean TPM counts ± SEM and analyzed using Two-tailed t-Test. *p<0.05 ***p<0.001 (C) Validation of upregulation of key ISGs using qRT-PCR. Data are represented as fold change n=3 biological replicates cultures ± SEM, normalized to mean of MOCK expression and analyzed using Two tailed t-test. *p<0.5, **p<0.01, ***p<0.001, ****p<0.0001.

FIG. 29, panels A-D, depicts expression of ACE2 and TMPRSS2 in exocrine pancreatic cells. A. iPan^{EXO} Ductal cells CK19 (red) exhibiting ACE2 (green) expression. B. iPan^{EXO} Acinar cells Amylase (red), MIST1 (gray) exhibit ACE2 (green) expression. C. iPan^{EXO} Acinar and iPan^{ENDO} cultures contain some endocrine C-peptide expressing cells, which also co-stain with ACE2. D. iPSC-derived pancreatic exocrine cells as well as human acinar tissues and human ductal cell line H6C7 express ACE2 and TMPRSS2. Data is shown as mean ± SEM with statistical significance determined by unpaired two-tailed t-test. *p<0.05, **p <0.01, ***p<0.001, ****p<0.0001. Scale bar represents 100 µm, and 20 µm for zoomed panels adjacent to main images.

FIG. 30, panels A-C, shows that SARS-CoV-2 can infect iPan^{EXO} Ductal Cells. A. Differentiation scheme of pancreatic ductal cells and infection of SARS-CoV-2 on Day 26. B. Immunocytochemistry staining of SARS-CoV-2 at Day 1 and Day 3 (Scale bar is 200 µm). C. RT-qPCR showing upregulation of SARS-CoV-2 from mock to infected cells at different days. Data is shown as mean ± SEM with statistical significance determined by unpaired two-tailed t-test. *p<0.05, **p <0.01, ****p<0.0001. Scale bar represents 200 µm.

FIG. 31, panels A-C, shows that SARS-CoV-2 elicits abnormal cellular phenotypes in iPan^{EXO} Ductal cultures. A. Day 1 and Day 3 of post infected iPan^{EXO} Ductal cells. Immunocytochemistry staining of ductal cell markers CK19 (red), SOX9 (gray), and SARS-CoV-2 infected cells (green) on Day 1 and Day 3 with varying titers of virus (Mock, MOI 0.05, and 0.1). B. SOX9 translocation in infected cells. (Scale bar is 200 µm). C. Histogram showing the ratio of cells with mis localized cytoplasmic SOX9 over total nuclear SOX9 positive cells in the culture, comparing Mock vs SARS-CoV-2 infected ductal cultures at MOI 0.05 and 0.1. Data is shown as mean ± SEM with statistical significance determined by unpaired two-tailed t-test. *p<0.05, **p <0.01. Scale bar represents 200 µm.Zoomed panels adjacent to main images are 2.3x larger.

FIG. 32, panels A-E, depicts that iPan^{EXO} Acinar cultures can be infected with SARS-CoV-2 and result in upregulation of some proinflammatory genes. A. Differentiation and infection timeline for the iPan^{EXO} Acinar cells. Cells were grown 16 days before being infected. Cells were fixed or lysed on the first and third days of infection. B. Immunocytochemistry staining of SARS-CoV-2 on Day 1 and 3 of Mock and infected cells. C. Quantification of immunocytochemistry images shows significant increase in SARS-CoV-2 positive cells in the population treated with the virus at MOI 0.1. D. qPCR shows upregulation of SARS-CoV-2 in infected cells. E and F. qPCR of inflammation markers CXCL12, NFKB, and STAT3 show significant upregulation between Mock and infected cells on day 3 of infection. Data is shown as mean ± SEM with statistical significance determined by unpaired two-tailed t-test. *p<0.05, **p<0.01, ***p<0.001. Scale bar represents 130 µm, and 10 µm for zoomed panels adjacent to main images.

FIG. 33, panels A-G, depicts RNAseq analysis of infected iPan^{EXO} cells on days 1 and 3 post infection. A. Principle Component Analysis of infected and mock samples collected on day 1 and day 3, respectively. B. Differentially Expressed Genes Heat map showing the relative expression levels of transcripts differentially expressed with adjusted p-values less than 0.01. C. Volcano plot of the log10 adjusted p-value of each expressed transcript on day 3 versus the log2 fold change. Transcripts that did not demonstrate differential expression with an adjusted p-value of less than 0.05 and a log2 fold-change in either direction greater than 0.5 are plotted in grey. Those that did were sized proportionally to their mean expression level, and genes of interest have been labeled. D. Gene Ontology terms in the Biological Process database were plotted based on the probability of the observed enrichment. The 500 most upregulated and the top 500 most downregulated transcripts were submitted to Enrichr. The fraction of genes in the gene set which were present in the 500 transcripts under analysis are listed in blue. E. Enrichment scores for the 500 most downregulated transcripts on day 3 when compared to the Gene Expression Omnibus’ gene sets of genes upregulated in response to viral perturbation. F. Enrichment scores for the most upregulated and downregulated transcripts on day 3 within the COVID-19-related Drug and Gene Set Library.

FIG. 34, panels A-F, shows that post-mortem human pancreas shows SARS-CoV-2 infectivity and co-localization with multiple pancreatic cell types. Immunohistochemistry results from pancreatic tissues of COVID-19 patient A20-32 show SARS-CoV-2 staining in green and pancreatic acinar markers in red, such as A. Chymotrypsin, B. Amylase; C. ductal marker cytokeratin 19 (CK19), D. CFTR; E. endocrine marker C-peptide in gray; and E. peripheral macrophage marker CD68 in red. F. Another set of pancreatic tissues from COVID-19 patients (n=5) and Control subjects (n=6) were utilized for RNA extraction. Real-time qPCR results show increased mRNA expression of ductal markers CA2, CFTR, KRT19 and CFTR. Data is shown as mean±SEM with statistical significance determined by unpaired two-tailed t-test. *p<0.05. Scale bar represents 50 µM.

FIG. 35 shows iPan^{EXO} organoids exhibit ACE2 expression. iPanEXO organoids exhibit polarization (E-cadherin), as well as markers of both pancreatic acinar (CTRC) and ductal cells (CK19, SOX9). These organoids exhibit ACE2 expression similar to adherent iPanEXO cultures. Scale bar represents 200 µm for large rectangle images, 50 µm for small square images, and 10 µm for zoomed panels adjacent to main images.

FIG. 36 shows that iPan^{EXO} ductal cells express ACE2. Day 3 post infected iPanEXO ductal cells exhibit ACE2 expression in SARS-CoV-2 infected and uninfected Mock control groups. The images below show the magnified regions of interest of DAPI and ACE2 staining in the dotted line boxes shown in the main panel for Mock and MOI 0.05 groups. Scale bar represents 200 µm.

FIG. 37 shows that C-peptide positive cells are infected by SARS-CoV-2. iPan^{EXO} acinar cultures contain small clusters of C-peptide protein expressing cells. A portion of these C-peptide positive cells were found to additionally express SARS-CoV-2 on Day 3 (72 hours post-infection). Scale bar represents 130 µm, and 20 µm for zoomed panels adjacent to main images.

FIG. 38, panels A and B, shows that inflammatory markers were not altered by SARS-CoV-2 infection at Day 1 and as well as IL1B and T FA at Day 3 A. RT-qPCR of infected and non-infected samples collected 24 hours (Day 1) after infection do not show significant change of mRNA expression of CXCL12, IL1B, TNFA, NFKB, or STAT3. B. RT-qPCR of infected and non-infected samples collected 72 hours (Day 3) after infection do not show significant change of IL1B or TNFA mRNA expression. Data is shown as mean ± SEM with statistical significance determined by unpaired two-tailed t-test.

FIG. 39, panels A-D, depicts Post-Mortem Human Pancreas Staining from other COVID-19 patients. Patients i. A20-32 and ii. A20-35 exhibit SARS-CoV-2 (green) co-stained with A. pancreatic acinar markers CTRC (red) and B. Amylase (red), C. pancreatic ductal markers CK19 (red) as well as D. pancreatic endocrine C-peptide (gray) with macrophage marker CD68 (red). Scale bar represents 50 µM. Scale bar in zoomed panels represents 25 µM.

FIG. 40, panels A and B, depicts Post-Mortem Human Pancreas from another other COVID-19 patient. Patient A20-3693 exhibits SARS-CoV-2 (green) co-stained with A. s A (red) and B. few areas of endothelial CD31 + cells. Scale bar represents 50 µM.

FIG. 41, panels A-C, depicts mRNA expression of additional pancreatic and inflammatory markers from post-mortem COVID-19 and Control patients. Post-mortem pancreatic tissues from COVID-19 patients (n=6) and Control patients (n=6) were probed for mRNA expression of A. endocrine markers NGN3 and INS; B. acinar markers CTRC, AMY1A, PTF1A and MIST1; and C. inflammatory markers IL1B, TNFA, CXCL12, NFKB1 and STAT3. Data is shown as mean ± SEM with statistical significance determined by unpaired two-tailed t-test.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the term “organ chip” (also referred to as “organ on chip”) refers to a microfluidic culture device are capable of recapitulating the microarchitecture and functions of living organs.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Modulation” or “modulates” or “modulating” as used herein refers to upregulation (i.e., activation or stimulation), down regulation (i.e., inhibition or suppression) of a response or the two in combination or apart.

“Pharmaceutically acceptable carriers” as used herein refer to conventional pharmaceutically acceptable carriers useful in this invention.

“Promote” and/or “promoting” as used herein refer to an augmentation in a particular behavior of a cell or organism.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Therapeutically effective amount” as used herein refers to the quantity of a specified composition, or active agent in the composition, sufficient to achieve a desired effect in a subject being treated. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

COVID-19 affects multiple organ systems resulting in tissue injury and organ failure. It is imperative to understand the susceptibility and tissue injury mechanisms of various tissue systems for SARS-CoV-2 virus infection in 2D, 3D and microfluidic CHIP culture platforms. As a result, one can develop susceptible human cell system for antiviral drug screening to treat COVID-19.

The Inventors have extensive expertise in tissue cell types, whether from primary or induced pluripotent stem cell (iPSC) sources, including organoids in fluidic, microfluidic and chip platforms. This includes lung, heart, endothelial cells, pancreatic cells or even use of exosomes derived from therapeutic cell types. Preliminary studies with lung and heart models are described herein.

Further information is found in U.S. App. No. 15/458,185, 15/352,289, PCT App. No. PCT App. No. PCT/US2017/49115, PCT App. No. PCT/US2017/49193, PCT App. No. PCT/US2017/16079, PCT App. No. PCT/US2017/16098, PCT App. No. PCT/US2017/16079, PCT App. No. PCT/US2017/16098, PCT App. No. PCT/US2017/016098, PCT App. No. PCT/US2017/16079, PCT App. No. PCT/US2018-022511, PCT App. No. PCT/US2016/57724, and PCT App. No. PCT/US2017/49115, PCT App. No. PCT/US2019/023749, in U.S. App. No. 62/243,642, 62/277,723, 62/332,727, 62/380,780, and 63/019,217 which are fully incorporated by reference herein.

COVID-19 caused by infection with the SARS-CoV-2 virus, is the most recent of a series of severe viral infections that typically initiate in the upper respiratory tract but have the potential to cause life-threatening pneumonia due to infection and inflammation of the lower respiratory tract. Unlike human coronaviruses that lead to a self-limiting upper respiratory tract infection, SARS-CoV-2 and related viruses SARS-CoV and MERS-CoV are thought to originate from bats and cause severe symptoms in their human hosts due to lack of host-pathogen adaptation. Similar zoonotic transmission of other respiratory pathogens, such as H1N1 influenza A virus are associated with recurrent pandemics with severe pulmonary complications seen among infected individuals due to infection of the lower respiratory tract. Even though acute viral pneumonia appears to be a common pathological outcome of infection with these severe respiratory viruses, mechanisms leading to these adverse outcomes are poorly understood. We sought to develop culture models of proximal and distal human lung epithelium as a platform to define disease mechanisms and for rapid drug discovery in the setting of current and future severe respiratory viral infections. Moreover, this study is the first report of a primary in vitro system of the adult human alveoli to model COVID-19.

Our study described herein provides an in vitro model to investigate infections such as SARS-CoV-2 infection along the proximal-distal axis of the human lung epithelium. Most of the current knowledge of SARS-CoV-2 infection of the lung conies from experiments in non-physiological cell lines that express angiotensin-converting enzyme2 (ACE2), the receptor for SARS-Co-V2 entry and attachment, such as Vero E6 cells or lung cell lines with or without exogenous expression of ACE2. However, these models do not replicate the complex physiology of the different polarized lung epithelial cell types. Moreover, commonly used animal models for preclinical drug screening such as mice are not natural hosts to SARS-CoV-2 infection. Therefore, there is an urgent need for development of model systems that can mimic the physiology and host response of the human lung and provide a relevant platform for screening potential therapeutic agents that target SARS-CoV-2 infection and replication.

More recently, some studies have utilized primary epithelial cultures of nasal and proximal airway epithelium and organ on chip methods to study SARS-CoV-2 infection of the upper airways, the initial site of entry and infection. We utilized the well differentiated ALI culture system to study SARS-CoV-2 infection of proximal airway epithelial cells and found that SARS-Co-V2 predominantly infected ciliated cells in addition to a sub-population of goblet cells. This observation is congruent with previous reports of infection of ciliated cells by SARS-CoV and recent reports of infection of ciliated cells by SARS-CoV-2, in in vitro cultures of proximal airways as well as COVID-19 patient biopsies (Hou et al., 2020). A number of studies employing single cell RNA sequencing analysis have described the expression of ACE2 in the respiratory tracts and in the nasal passages and large airways, highest levels of ACE2 expression have been reported in ciliated cells. Although proximal airways are the initial site of SARS-CoV-2 attachment and infection, it is infection and inflammation of the distal lung that drives the severe and fatal symptoms in COVID-19 among highly susceptible patients. Recent single cell transcriptome studies have shown that ACE2, is predominantly expressed by AT2 cells. AT2 cells are bifunctional cells that serve as facultative progenitors, contributing to epithelial maintenance in addition to fulfilling specialized functions such as surfactant production. In this study we describe the development of an in vitro model of primary AT2 cells isolated from the adult human lung. In our model, AT2 were readily infected by SARS-CoV-2 and demonstrated a significant upregulation of heat shock proteins and chaperons inducing cellular apoptosis. Moreover, our model demonstrated cell autonomous and non-cell autonomous apoptosis induced by viral infection which may contribute to alveolar injury seen in COVID-19. Infection of AT2 cells was accompanied by a cell-intrinsic proinflammatory response and upregulation of the interferon signaling pathway, characterized by upregulation of type 1 and type 3 interferon genes as well as several interferon signaling genes. Recent studies have reported that interferon response is diminished or delayed in severe cases of COVID-19, similar to that seen in severe cases of MERS and SARS-CoV. However, we observed a moderate interferon response in our model cells after SARS-Co-V2 infection. A recently published report of SARS-CoV-2 infection of pluripotent stem cell-derived AT2 cells also demonstrated an induction of interferon signaling response similar to our model of primary adult AT2 cells (Huang et al., 2020). The presence of an IFN response in cells derived from normal donors may explain the lack of a severe phenotype in a large proportion of COVID-19 patients. It will be of interest to evaluate the host response to SARS-CoV-2 infection in primary AT2 cells derived from donors with pre-existing lung conditions in future. It is also important to note that our model system does not consist of an immune cell component. We speculate that the epithelial innate immune response may provide activating signals leading to global activation of the host immune response and provide therapeutic targets for mitigation of uncontrolled lung inflammation and adverse patient outcomes. To better understand host immune response to SARS-CoV-2 infection, co-culture systems of infected AT2 cells with immune cells can be developed in future.

Further we tested the efficacy of selected potential therapeutic agents against SARS-CoV-2 infection of AT2 cells and found that viral infection and/or replication was strongly suppressed by the candidate drug, Remdesivir. Remdesivir is currently the most promising candidate drug and has been granted emergency use authorization for treatment of hospitalized COVID-19 patients by the FDA. Type 1 interferons are known antiviral agents and IFNB1 has been suggested to reduce SARS-CoV-2 infection in Vero Cells and more recently in patients with severe COV1D-19 in an ongoing clinical trial (NCT04385095). Consistent with these findings, we also observed significant suppression viral infection upon treatment of AT2 cultures with IFNB1. Thus, our platform provides a relevant model to effectively screen and validate drugs targets against SARS-Co-V-2. Infection of AT2 cells drives acute respiratory distress syndrome in severe cases of COVID-19. We sought to develop culture models of proximal and distal human lung epithelium as a platform to study early response to SARS-CoV-2 infection and for rapid drug discovery in the setting of current and future severe respiratory viral infections.

With respect to pancreatic cells the inventors have developed and validated in our laboratory novel methods to generate pancreatic progenitors from human induced pluripotent stem cells (iPSCs), which can be fated into endocrine (β-cells) and exocrine (acinar and ductal) cells. These cultures contain cells that are representative of human endocrine pancreas expressing NKX6.1 and C-peptide positive cells and exocrine pancreas with expression of Amylase and Chymotrypsin (CTRC) (iPanEXO Acinar cells) and Cytokeratin19 (CK19) and SOX9 (iPanEXO Ductal). Here, the use of these iPSC-derived pancreatic cells was employed to dissect the effects of SARS-CoV-2 infection. We show for the first time that iPSC-derived pancreatic cells including endocrine (iPanENDO) and exocrine cell types (iPanEXO Acinar and Ductal cells) are susceptible to SARS-CoV-2 infection, resulting in morphological alterations as well as impaired expression of key markers. Importantly, these cellular phenotypes occurred in the presence of inflammatory impacts. These observations were confirmed in post-mortem pancreatic tissues from COVID-19 patients. Thus, these results suggest the pancreatic cells may be susceptible to infection by SARS-CoV-2, and an iPSC-based model provides a valuable new platform for understanding the pancreas-specific cellular responses to SARS-CoV-2 infection as well as for antiviral drug testing against SARS-CoV-2.

The main symptoms described by COVID-19 patients are related to the respiratory tract, such as cough, dyspnea, pneumonia, sore throat and acute respiratory distress syndrome. However, nearly one quarter of COVID-19 patients present with other symptoms not-related to the respiratory tract but related to the gastrointestinal system, such as diarrhea, nausea, vomiting, and abdominal pain, irrespective of respiratory perturbations, which suggests that the virus may not affect solely the respiratory tract. Many other patients are asymptomatic. Interestingly, some of those who report abdominal pain also present elevated levels of serum pancreatic enzymes, such as amylase and lipase, accompanied or not with an enlargement of the pancreas or dilatation of the pancreatic duct, which are acute pancreatitis-like symptoms. Since most of the studies exploring the association of the viral infection with abnormalities of the pancreas are clinical and observational, we were interested in learning more about this taking advantage of the iPSC-derived pancreatic cells, including effects on exocrine cells (acinar and ductal), which have not been explored in the published studies.

In this study, the seminal observation was that human iPSC-derived pancreatic cultures containing endocrine, acinar and ductal cells show ACE2 expression, both at protein and mRNA levels. Importantly, ACE2 expression is co-localized with endocrine, acinar, and ductal markers (CK19 and SOX9). It is now accepted that SARS-CoV-2 enters the cells through ACE2/TMPRSS2 entry; thus the likely reason that our iPSC-derived pancreatic cultures are permissive for SARS-CoV-2 infection. Interestingly, ACE2 expression was also present in cultures of primary human acinar cells as well H6C7 human ductal cell line. Other studies have previously shown that human pancreas highly express ACE2, mainly in the exocrine portion. When infected with SARS-CoV-2, the iPSC-derived pancreatic cultures rapidly uptake the virus within 24 and 72 hours, as demonstrated by immunocytochemistry and mRNA expression of the virus. The presence of the virus was detected in pancreatic endocrine, exocrine (ductal and acinar) cells. To the best of our knowledge, this is the first time that human iPSC-derived pancreatic cultures containing exocrine cells have been shown to be capable of being infected by SARS-CoV-2.

Specifically, compared to the uninfected mock controls, iPan^{EXO} acinar infected cultures had significantly higher levels of SARS-CoV-2. It has been previously reported that a common feature of coronaviruses is the use of virus-engineered double membrane vesicles are a central site for viral RNA synthesis. Granular morphology was noted in all SARS-CoV-2 positive cells and could be representative of viral replication and transportation occurring in vesicles throughout the cells, though more in-depth analysis is needed to confirm this. Immunocytochemistry staining showed co-staining of SARS-CoV-2 in Chymotrypsin C positive cells, implying infection of acinar cells specifically. No major changes in cell morphology or density were noted, however upregulation of pancreatic proinflammatory gene CXCL12 was observed. Previous studies have found evidence for SARS-CoV-2 infection inducing a cytokine storm, the body’s overproduction of pro-inflammatory markers. Increased circulating levels of chemokines and cytokines, including CXCL10, CXCL9, and IL1B have been seen in COVID-19 patients. Similarly, upregulation of CXCL12 by more than 10-fold has been found when examining the plasma of COVID-19 patients as opposed to healthy patients.

SARS-CoV-2 also was successful in infecting the iPan^{EX0} Ductal cultures, with a subset of SARS-CoV-2 positive cells co-staining with ductal markers CK19 and SOX9. Interestingly, the morphology of infected ductal cells differed distinctly from that of the uninfected ductal cells. The nuclear compartmentalization of SOX9, which is typically observed in ductal cells was absent, and replaced with a cytoplasmic staining pattern, suggesting a nuclear mislocalization of SOX9 as a potential indicator of cellular stress imposed by the virus in infected cells. Chakravarty et al. reported that the cytoplasmic accumulation of SOX9 was correlated with increased cell proliferation of invasive carcinoma in breast tissue (Chakravarty et al., 2011). Other studies have also reported the presence of SOX9 in an array of cancers such as breast carcinoma, lung adenocarcinoma, ovarian cancer and prostate cancer. In cancer cells, SOX9 seems to play a key role in controlling ECM, cell adhesion and remodeling of the cytoskeleton during tumor invasion. Further, these infected ductal cells also have markedly enlarged/elongated morphology, a feature that is also common in cancerous or inflamed epithelial cells converting to fibrotic cells undergoing a dynamic process known as epithelial-mesenchymal transition (EMT). Based on the findings, further elucidation of the mechanisms involved in response to the SARS-CoV-2 infection in pancreatic ductal cells is required which could perhaps explain similar cellular responses in other pancreatic injury models.

Next generation RNA-sequencing analysis of infected iPSC-derived pancreatic cultures show a marked difference in gene expression between the infected and uninfected cultures. Clear separation of infected and uninfected samples could be observed as soon as Day 1 (24 hours) which was amplified at Day 3 post infection. Among the upregulated biological processes in the Day 3 post-infection samples are SRP-dependent protein-targeting processes, viral gene transcription and translation, and nonsense-mediated mRNA decay. The viral replication pathway upregulation implies that not only infection, but active replication of SARS-CoV-2 is occurring. As mentioned previously, studies have found that SARS-CoV makes use of host cell components, such as endoplasmic reticulum and cell membrane, to create double membrane vesicles for replication. The upregulation of SRP-protein targeting processes could be a reflection of host cell machinery being repurposed for viral replication. It has additionally been found with murine hepatitis virus, a murine coronavirus, that the nonsense-mediated mRNA decay pathway plays a role in the degradation of CoV mRNA early in infection. Our results could be indicative of an early host defense response.

Upregulated cellular components in infected cells are primarily ribosomal and cytosolic. This is in agreement with previous findings that SARS-CoV-2 associates with the host endomembrane system, with 40% of SARS-CoV-2-interacting proteins having functions in the endomembrane system. SARS-CoV-2 protein translation, assembly, and release necessitates interaction with endomembrane components and could therefore lead to their upregulation. Conversely, the top three downregulated cellular components are related to the nucleus. SARS-CoV nucleocapsid proteins (N) are found to localize to the nucleolus. Computational modeling for RNA localization suggests SARS-CoV-2 has even stronger nuclear localization than SARS-CoV, and previous research has found evidence that SARS-CoV-2 proteins, including Nsp5, Nsp9, and Nsp 15, interact with components of the nucleus. Of note, many important cellular processes related to cell replication and cytokinesis occur in the nucleus. Other coronaviruses, including avian infectious bronchitis virus and murine hepatitis virus, have been found to have rolls in cell cycle arrest, with evidence showing arrest induction leads to increased viral replication. SARS-CoV protein ORF7a has been found to reduce cyclin D3, which halts the cell cycle at the G0/G1 phase. Our results for the most upregulated and downregulated transcripts within the COVID-19-related Drug and Gene Set Library align with that of other coronaviruses, particularly SARS-CoV and MHV, with the top two downregulated transcripts being SARS-CoV-2 related. Taken all together, our results suggest that SARS-CoV-2, like other coronaviruses, elicits similar transcript-level signatures to promote viral replication in pancreatic cells. Future studies are necessary to confirm these mechanistic implications of these findings.

Past studies have primarily focused on susceptibility of in vitro cultured cells to SARS-CoV-2 infection without always confirming findings in human COVID-19 patient organs and tissues. Post-mortem human pancreata from COVID-19 patients was utilized here to further confirm observations in cell culture. Pancreatic tissues from COVID-19 patients showed SARS-CoV-2 was co-localized with AMY⁺ and CTRC⁺ acinar cells, CK19⁺ and CFTR⁺ ductal cells and C-peptide⁺ endocrine β-cells, confirming that this novel coronavirus is capable of infecting both exocrine and endocrine pancreatic cells. The co-infection with C-peptide⁺ islet clusters is particularly interesting given that diabetes and diabetes-related complications, glycemic control and high body mass index are associated with high mortality rates related to COVID-19, and are considered independent risk factors. In this study, male sex, older age, renal impairment, non-white ethnicity, socioeconomic deprivation, and previous stroke and heart failure were associated with increased COVID-19-related mortality in both T1D and T2D patients.

While not wishing to be bound by any particular theory, we believe that pancreas is likely not the primary site of direct infection by SARS-CoV-2 and instead the virus may be entering the pancreas through the peripheral circulatory system via macrophages. Previous studies have shown that certain viruses use peripheral monocytes/macrophages as reservoirs for productive self-replication, thus favoring virus transmission and target tissue dissemination. In the post-mortem pancreatic tissue from COVID-19 patients, we observed evidence of SARS-CoV-2 co-localizing with CD68, which is a marker for peripheral macrophages, specifically in close association with some C-peptide⁺ pancreatic islet clusters that were also positive for SARS-CoV-2. This suggests that SARS-CoV-2 may enter the pancreas peripherally through infected monocytes/macrophages in circulation that could have migrated to the pancreas as a result of a dysregulated macrophage response. Such observations are supported by reports in previous studies. Since both exocrine and endocrine pancreatic cells express ACE2, as shown by our study and others, these cells could have been a target for the virus presented by infected macrophages. Infectivity of macrophages by SARS-CoV-2 has been shown by previous studies, which may be through ACE2-dependent and ACE2-independent pathways. The route of SARS-CoV-2 entry into the pancreas remains an interesting question and future studies should explore this in greater detail.

To begin understanding the effects of SARS-CoV-2 infectivity in the human pancreas, we also interrogated perturbations in several endocrine, exocrine and inflammatory genes from post-mortem tissue of COVID-19 and non-COVID-19 control subjects. Notably, important ductal genes were upregulated in the samples from COVID-19 patients, such as CK19 (Cytokeratin 19; KRT19 gene), CA2 and CFTR. It is known that KRT19 overexpression is associated with intracellular stress conditions of the pancreas, such as reported by in patients with poor prognosis of pancreatic ductal adenocarcinoma (PDAC). Cytoskeletal proteins, which consist of different subfamilies of proteins including microtubules, actin and intermediate filaments, are essential for survival and cellular responses. Their response to important intracellular stress such as mitochondrial, endoplasmic reticulum and oxidative stress can be dysregulated. In addition, cytoskeletal responses have been reported to be an important agent in both immune and adaptive immunity.

Overall, we show SARS-CoV-2 can infect pancreatic endocrine and exocrine cells in cultured cells as well as in an intact organ of a human patient, thus leading to abnormal molecular and cellular phenotypes. Our results are consistent with previously reported mechanisms of SARS-CoV-2 and SARS coronaviruses in other cell types, and suggest that SARS-CoV-2 can adversely impact the pancreas. This study supports the utility of iPSC-derived pancreatic cells as an excellent model system to explore the detrimental impacts of SARS-CoV-2 on pancreatic cells. With this study, we provide a novel, patient-specific model for future studies of SARS-CoV-2 on the pancreas.

As such, various embodiments of the present invention are based, at least in part, by these findings.

Various embodiments of the present invention provide for a system for infection modeling or test agent screening, comprising: a population of cells comprising cells selected from the group consisting of progenitor cells derived from induced pluripotent stem cells (iPSCs), cells differentiated from iPSCs, an organoid comprising the cells differentiated from iPSCs, or primary cells; a fluidic device, a cell culture plate, or a multi-well culture plate; wherein an infectious agent and the population of cells are in contact in the fluidic device, the cell culture plate, or the multi-well culture plate.

Various embodiments of the present invention provide for a system for infection modeling or test agent screening, comprising: a population of cells comprising cells selected from the group consisting of progenitor cells derived from induced pluripotent stem cells (iPSCs), cells differentiated from iPSCs, an organoid comprising the cells differentiated from iPSCs, or primary cells; a fluidic device, a cell culture plate, or a multi-well culture plate; wherein an infectious agent, a test agent, and the population of cells, are in contact in the fluidic device, the cell culture plate, or the multi-well culture plate.

In various embodiments, the fluidic device is an air-liquid interface culture. In various embodiments, the fluidic device is a Transwell system comprising the population of cells.

In various embodiments, the fluidic device is a microfluidic device comprising the population of cells. In various embodiments, the microfluidic device is an organ chip.

In various embodiments, wherein the fluidic device, the cell culture plate, or the multi-well culture plate comprises an air-liquid interface culture, Transwell system, microfluidic device or an organ chip, the population of cells comprises progenitor cells derived from induced pluripotent stem cells (iPSCs) selected from progenitor heart cells or progenitor endothelial cells.

In various embodiments, wherein the fluidic device, the cell culture plate, or the multi-well culture plate comprises an air-liquid interface culture, Transwell system, or a microfluidic device, the population of cells comprises progenitor cells derived from induced pluripotent stem cells (iPSCs) selected from progenitor lung cells or progenitor pancreatic cells.

In various embodiments, the lung cells are epithelial cells. In various embodiments, the epithelial cells are proximal airway cells, or distal alveolar cells. In various embodiments, the heart cells are cardiac myocytes.

In various embodiments, the population of cells comprises cells differentiated from iPSCs. In various embodiments, wherein the fluidic device, the cell culture plate, or the multi-well culture plate comprise an air-liquid interface culture, Transwell system, or a microfluidic device, or organ chip, the population of cells comprises the cells differentiated from iPSCs comprise heart cells and endothelial cells.

In various embodiments, the population of cells comprises cells differentiated from iPSCs. In various embodiments, wherein the fluidic device, the cell culture plate, or the multi-well culture plate comprises an air-liquid interface culture, Transwell system, or a microfluidic device, the population of cells comprises the cells differentiated from iPSCs comprise lung cells or pancreatic cells.

In various embodiments, wherein the fluidic device, the cell culture plate, or the multi-well culture plate comprises an air-liquid interface culture, Transwell system, or a microfluidic device, or organ chip, the population of cells comprises an organoid comprising the cells differentiated from iPSCs selected from heart cells or endothelial cells. In other examples, population of cells comprises an organoid comprising heart cells (e.g., cardiac myocytes).

In various embodiments, wherein the fluidic device, the cell culture plate, or the multi-well culture plate comprises an air-liquid interface culture, Transwell system, or a microfluidic device, the population of cells comprises an organoid comprising the cells differentiated from iPSCs selected from lung cells or pancreatic cells. In other examples, the population of cells comprises an organoid comprising the epithelial cells (e.g., proximal airway cells, or distal alveolar cells).

In various embodiments, the population of cells comprises primary cells. In various embodiments, wherein the fluidic device, the cell culture plate, or the multi-well culture plate comprises an air-liquid interface culture, Transwell system, microfluidic device or an organ chip, the primary cells comprise heart cells or endothelial cells. In various embodiments, the heart cells are cardiac myocytes.

In various embodiments, the population of cells comprises primary cells. In various embodiments, wherein the fluidic device, the cell culture plate, or the multi-well culture plate comprises an air-liquid interface culture, Transwell system, or microfluidic device, the primary cells comprise lung cells or pancreatic cells. In various embodiments, the lung cells are epithelial cells. In various embodiments, the epithelial cells are proximal airway cells, or distal alveolar cells.

In various embodiments, the infectious agent is a virus, a bacterium, a fungus, or a parasite. In various embodiments, the virus is a coronavirus. In various embodiments, the coronavirus is SARS-CoV-1, MERS, or SARS CoV-2.

In various embodiments, the test agent is an anti-viral agent. In various embodiments, the test agent is an antibacterial agent. In various embodiments, the test agent is an antifungal agent. In various embodiments, the test agent is an anti-parasitic agent. In various embodiments, the test agent is a nucleotide analog, an anti-inflammatory agent, interferon beta, ribavirin, chloroquine, azithromycin, favipiravir, lopinavir/ritonavir or remdesivir.

Various embodiments of the present invention provide for a method selecting an agent of interest, comprising: contacting a test agent with a population of cells comprising cells selected from the group consisting of progenitor cells derived from induced pluripotent stem cells (iPSCs), cells differentiated from iPSCs, an organoid comprising the cells differentiated from iPSCs, or primary cells, wherein the test agent and the population of cells are in contact in a fluidic device, a cell culture plate, or a multi-well culture plate; infecting the population of cells with an infectious agent before, simultaneously or after contacting the test agent with the population of cells; measuring a parameter in the population of cells; and selecting the test agent as the agent of interest based on the measured parameter in the population of cells.

Various embodiments provide for a method of studying an infectious agent, comprising: contacting an infectious agent with a population of cells comprising cells selected from the group consisting of progenitor cells derived from induced pluripotent stem cells (iPSCs), cells differentiated from iPSCs, an organoid comprising the cells differentiated from iPSCs, or primary cells, wherein the infectious agent and the population of cells are in contact in a fluidic device, a cell culture plate, or a multi-well culture plate; and measuring a parameter in the population of cells.

In various embodiments, the parameter comprises a phenotype of interest, an expression level of a gene, or an expression level of a protein of interest, or combination thereof

In various embodiments, the fluidic device is an air-liquid interface culture. In various embodiments, the fluidic device is a Transwell system comprising the population of cells.

In various embodiments, the fluidic device is a microfluidic device comprising the population of cells. In various embodiments, the microfluidic device is an organ chip.

In various embodiments, the population of cells comprises progenitor cells derived from induced pluripotent stem cells (iPSCs). In various embodiments, the progenitor cells derived from induced pluripotent stem cells (iPSCs) comprise progenitor lung cells, progenitor heart cells, progenitor endothelial cells, or progenitor pancreatic cells. In various embodiments, the lung cells are epithelial cells. In various embodiments, the epithelial cells are proximal airway cells, or distal alveolar cells. In various embodiments, the heart cells are cardiac myocytes.

In various embodiments, the population of cells comprises cells differentiated from iPSCs. In various embodiments, the cells differentiated from iPSCs comprise lung cells, heart cells, endothelial cells, or pancreatic cells.

In various embodiments, the population of cells comprises an organoid comprising the cells differentiated from iPSCs. For example, an organoid comprising lung cells, heart cells, endothelial cells, or pancreatic cells. In other examples, an organoid comprising the epithelial cells (e.g., proximal airway cells, or distal alveolar cells). In other examples, an organoid comprising heart cells (e.g., cardiac myocytes).

In various embodiments, the population of cells comprises primary cells. In various embodiments, the primary cells comprise lung cells, heart cells, endothelial cells, or pancreatic cells. In various embodiments, the lung cells are epithelial cells. In various embodiments, the epithelial cells are proximal airway cells, or distal alveolar cells. In various embodiments, the heart cells are cardiac myocytes.

In various embodiments, the infectious agent is a virus, a bacterium, a fungus, or a parasite. In various embodiments, the virus is a coronavirus. In various embodiments, the coronavirus is SARS-CoV-1, MERS, or SARS CoV-2.

In various embodiments, the test agent is an anti-viral agent. In various embodiments, the test agent is an antibacterial agent. In various embodiments, the test agent is an antifungal agent. In various embodiments, the test agent is an anti-parasitic agent. In various embodiments, the test agent is a nucleotide analog, an anti-inflammatory agent, interferon beta, ribavirin, chloroquine, azithromycin, favipiravir, lopinavir/ritonavir or remdesivir.

In various embodiments, the parameter comprises a phenotype of interest, expression level of a gene of interest, expression level of a protein of interest, or combinations thereof in the population of cells.

Various embodiments of the present invention provide for a method of selecting an agent of interest, comprising: contacting a test agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; measuring a phenotype of interest or expression level of a gene or protein of interest in the population of cells; selecting the test agent as the agent of interest based on the measurement of the phenotype of interest or expression level of the gene or protein of interest.

Various embodiments of the present invention provide for a method of selecting an agent of interest, comprising: contacting a test agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; infecting the population of cells with an infectious agent before, simultaneously or after contacting the test agent with the population of cells; and measuring a phenotype of interest or expression level of a gene or protein of interest in the population of cells; selecting the test agent as the agent of interest based on the measurement of the phenotype of interest or expression level of the gene or protein of interest. In various embodiments, the infecting the population of cells with an infectious agent is done before contacting the test agent with the population of cells. In various embodiments, the infecting the population of cells with an infectious agent is done simultaneously with contacting the test agent with the population of cells. In various embodiments, the infecting the population of cells with an infectious agent is done after contacting the test agent with the population of cells.

Various embodiments of the present invention provide for a method of studying an infectious agent, comprising: contacting an infectious agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; and measuring a phenotype of interest or expression level of a gene or protein of interest in the population of cells.

In various embodiments, the test agent and the population of cells are in contact in a fluidic device, or a cell culture plate, or a multi-well culture plate. In various embodiments, the fluidic device, or the cell culture plate, or the multi-well culture plate is a transwell system. In various embodiments, the fluidic device is a microfluidic device. In various embodiments, the microfluidic device is an organ chip.

In various embodiments, the population of cells selected are from the group consisting of pancreatic progenitor cells derived from induced pluripotent stem cells (iPSCs), pancreatic ductal cells derived from iPSCs, pancreatic acinar cells derived from iPSCs, an organoid comprising derived from iPSCs, an organoid comprising pancreatic acinar cells derived from iPSCs, an organoid comprising pancreatic ductal cells derived from iPSCs, and combinations thereof.

In various embodiments, the population of cells selected are from the group consisting of pancreatic ductal cells derived from pancreatic progenitor cells, pancreatic acinar cells derived from pancreatic progenitor cells, an organoid comprising pancreatic acinar cells derived from pancreatic progenitor cells, an organoid comprising pancreatic ductal cells derived from pancreatic progenitor cells, and combinations thereof.

In various embodiments, the infectious agent is a virus.

In various embodiments, the infectious agent is a coronavirus. In various embodiments, the infectious agent is SARS-CoV-2.

In various embodiments, the infectious agent is hepatotropic virus, Coxackie virus, cytomegalovirus (CMV), human immunodeficiency virus (HIV), herpes simplex virus (HSV), mumps, varicella-zoster virus, or hepatitis.

In various embodiments, the infectious agent is a bacterium. In various embodiments, the bacterium is mycoplasma, legionella, salmonella, and leptospira.

In various embodiments, the infectious agent is a parasite. In various embodiments, the infectious agent is toxoplasmosis, cryptosporidium, ascaris, lumbricoides, Fasciola hepatica, or E. granulosus tapeworm.

In various embodiments, the infectious agent is a fungus. In various embodiments, the infectious agent is aspergillus.

In various embodiments, the test agent is selected from the group consisting of interferon beta, ribavirin, chloroquine, azithromycin, favipiravir, lopinavir/ritonavir, and remdesivir. In various embodiments, the test agent is an antibody.

In various embodiments, the phenotype of interest or expression level of the gene or protein of interest can be for example, an inflammatory marker, or proinflammatory gene.

In these embodiments, the cells can be studied and candidate drugs can be tested for their effects on the cells. Further, the mechanism of infection and the resulting cascade of pathway activation or deactivation brought on by the infectious agents can be studied.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Differentiation Protocol

Human lung tissue was obtained from deceased tissue donors in compliance with consent procedures developed by International Institute for the Advancement of Medicine (IIAM) and approved by the Cedars-Sinai Medical Center Internal Review Board.

### Tissue Processing for Isolation of Lung Cells From Either Tracheo-Bronchial or Small Airway/Parenchymal (Small Airways and Alveoli) Regions

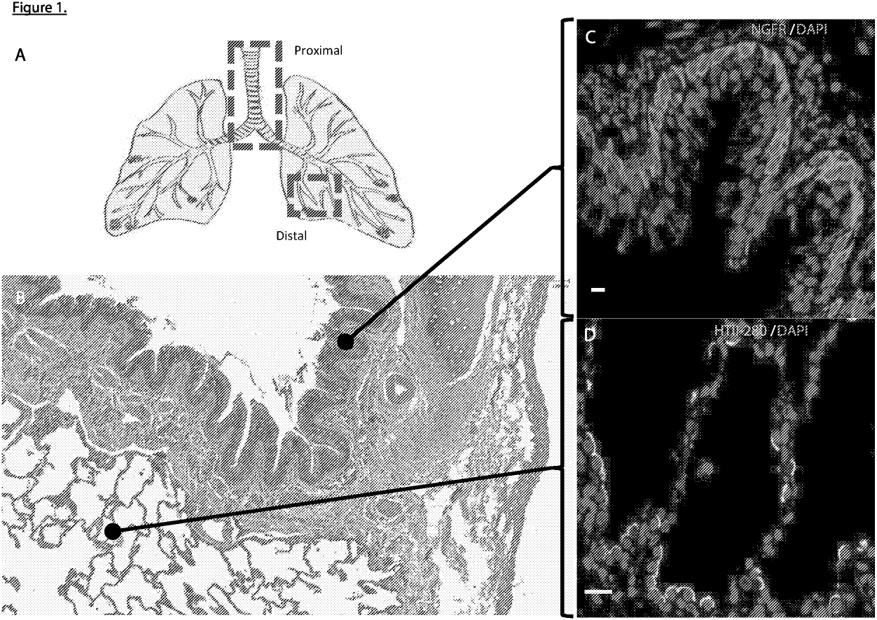

Prepare and autoclave all dissection instruments, glassware and the appropriate solutions one day prior to cell isolation. Upon receiving lung tissue identify and separate the proximal and distal regions as shown in the video. Briefly, the trachea along with the bronchi up to the first branching was considered ‘proximal’ and small airways (>2 mm in diameter) along with the surrounding tissue were considered ‘distal’ (FIG. 1A)

### Enrichment and Sub Setting of Small Airway and Alveolar Epithelial Progenitor Cells From Distal Lung Tissue

### Distal Tissue Preparation

Place distal lung tissue in a sterile petri dish (150×15 mm) in a biosafety cabinet. Dice tissue into approximately 1 cm³ pieces and place in a clean 50 mL tube. Wash tissue three times with chilled HBSS, discarding the HBSS wash each time to remove blood and epithelial lining fluid.

Place tissue in a new petri dish and blot dry with sterile Kim wipes. Using forceps and scissors, remove as much visceral pleura as possible. Use scissors to mince tissue into pieces of approximately 2 mm diameter.

Transfer minced tissue into a clean petri dish and mince further by chopping to an approximate size of 1 mm with a sterile single sided razor blade.

### Enzyme Digestion

Note: Liberase™ stock solution is 5 mg/mL (100×) and DNase stock is 2.5 mg/mL (100×).

Add 50 µg/mL Liberase™ and 25 µg/mL DNase solution solutions to sterile HBSS in a 50 mL conical tube.

Transfer minced tissue to a 50 mL conical tube and incubate for 40-60 minutes at 37° C. with continuous shaking using a Thermomixer set at 900 rpm.

Note: Incubation time in the enzymes can vary depending on the type or condition of the tissue. For example, enzymatic digestion of normal tissue takes approximately 45 minutes. However fibrotic tissue from IPF samples can require a longer incubation time of up to 60 minutes. Therefore, tissue should be carefully monitored during this step to prevent damage to the surface markers which is crucial for FACS and incubation times should be standardized based on the tissue type.

### Single Cell Isolation

Triturate tissue by drawing 5 times through a 16 G needle fitted to a 60 mL syringe. Draw tissue suspension into a wide-bore pipette and pass through a series of PluriSelect cell strainers (500 µm, 300 µm, 100 µm, 70 µm, 40 µm) under vacuum pressure. Wash the strainer with 20 mL of HBSS+ buffer to collect remaining cells. The recipe for HBSS+ buffer can be found in Table 2.

Add an equal volume of HBSS+ buffer to the filtrate to inhibit Liberase activity and prevent over-digestion.

Centrifuge filtrates at 500 × g for 5 minutes at 4° C.

Carefully remove and discard the supernatant. Add 1 mL of Red Blood Cell (RBC) lysis buffer to the pellet, gently rock the tube to dislodge the pellet and incubate on ice for 1 minute.

Note: The amount and time in the RBC lysis solution depends upon the size of the pellet. It is important to maintain the cells on ice and monitor time in RBC lysis solution carefully to prevent lysis of target cells. If RBC lysis is insufficient, the Inventors repeat the step.

Add 10-20 mL HBSS+ buffer to neutralize RBC lysis buffer. Centrifuge filtrates at 500 × g for 5 minutes at 4° C.

Note: Lysed red blood cells (ghost cells) may form a cloudy layer above the cell pellet. In this case, resuspend pellet in 10 mL of HBSS+ buffer and strain the suspension through 70 µm cell strainer to eliminate the ghost cells. Centrifuge filtrate at 600 × g for 5 minutes at 4° C. and proceed with further steps.

### Depletion of Immune Cells, Endothelial Cell (Optional Step)

Deplete CD31⁺ endothelial cells and CD45⁺ immune cells from the pool of total cells using the Milteny MACS CD31 & CD45 microbeads conjugated to monoclonal anti-human CD31 and CD45 antibody (isotype mouse IgG1) and LS columns in accordance to the manufacturer’s protocol.

Collect flow through consisting primarily of epithelial and stromal cells in a fresh sterile tube and centrifuge it at 600 × g for 5 mins at 4° C. Perform a cell count to ascertain the total number of cells in the flow through.

### Cell Surface Staining for Fluorescence Associated Cell Sorting (FACS)

Resuspend 1 × 10⁷ cells per 1 mL of HBSS+ buffer. Add primary antibodies at the required concentration and incubate the cells for 30 mins at 4° C. in dark. In this study, fluorophore conjugated primary antibodies were used unless otherwise stated. Details of antibody sources and titers are described in Table 5.

Note: HTII-280 is currently the best surface reactive Ab that allows subsetting of distal lung cells into predominantly airway (HTII-280⁻) and alveolar (HTII-280⁺) fractions. A caveat to this strategy is that AT1 cells are not stained using this method. However, AT1 cells are poorly represented in distal lung preps, presumably due to their fragility and loss during selection of viable cells by FACS and thus only represent a rare contaminant of the airway cell fraction).

Wash cells by adding 3 mL of HBSS+ buffer and centrifuge at 600 × g for 5 minutes at 4° C.

If using unconjugated primary antibodies, add required concentration of an appropriate fluorophore conjugated secondary antibody and incubate for 30 mins on ice.

Wash off excess secondary antibody by adding 3 mL of HBSS+ buffer and centrifuge at 600 × g for 5 mins at 4° C.

Discard supernatant and resuspend cells in HBSS+ buffer per 1 × 10⁷ cells/ mL.

Filter cells into 5 mL polystyrene tubes through a strainer cap to ensure formation of a single cell suspension.

Add DAPI (1 µg/mL) to stain permeable (dead) cells.

Note: It is essential to use appropriate single-color and Fluorescence minus one (FMO) controls (i.e. antibody staining cocktail minus one antibody each), to minimize false positives during FACS. In this study the Inventors use positive and negative selection beads for empirical compensation for overlap of emission spectra between fluorophores (Table 5).

Note: FACS enrich cell types of interest. Viable epithelial cells are enriched based upon their CD45-negative, CD31-negative, CD236-positive cell surface phenotype and negative staining for DAPI. This epithelial cell fraction can be further subsetted based on staining for cell type-specific surface markers, such as specific staining for HTII-280-positive cells that are enriched for AT2 cells. In contrast, negative selection for HTII-280 allows the enrichment of small airway epithelial cells such as club and ciliated cells.

### Enrichment and Subsetting of Epithelial Progenitor Cells From Tracheo-Bronchial Airways

### Tissue Preparation

Dissect out proximal airways (trachea/bronchi) from the lungs. Open airways along their length using scissors to expose the lumen and add 50 µg/mL Liberase™ to fully cover the tissue. Incubate for 20 minutes at 37° C. with continuous shaking using a Thermomixer set at 900 rpm.

Remove proximal airway from centrifuging tube and place it in a sterile petri dish (150×15 mm). Gently scrape the surface of the airway using a scalpel to completely strip luminal epithelial cells from the tissue. Wash the petri dish with a sterile 5 mL of HBSS+ buffer to collect all dislodged luminal epithelial cells and transfer the dislodged cells to 50 mL Falcon tube.

Triturate suspension by drawing 5 times through 16 G needle and 18 G needle fitted to a 10 mL syringe to get single cell suspension.

Centrifuge suspension at 500 × g for 5 mins at 4° C. Resuspend the pellet in a fresh HBSS+ buffer and store these luminal airway cells on ice, ready to be combined with the single cell suspension generated from the minced proximal airways in the further steps.

Using scissors, cut remaining tracheobronchial tissue along its rings to generate small strips of tissue and transfer the strips to a fresh petri dish.

Mince the tissue strips using a single sided razor blade to make smaller pieces. NOTE: Since the proximal airways are cartilaginous, they cannot be minced as finely as finely as the distal lung tissue.

Transfer minced tissue into the Gentle MACS C tubes, add 2 mL of Liberase™ to the tube ensuring that the tissue is submerged.

Load the C tube onto the Gentle MACS Octo Dissociator (Table 3) and run Human Lung Protocol-2 to mechanically dissociate tissue further.

Note: MACS Octo Dissociator offers an optimized gentle MACS program called human lung protocol-2 for this specific application.

### Enzyme Digestion and Single Cell Isolation

Transfer approximately 2 gm of minced proximal tissue from the C tube into each 50 mL Falcon tube and add 50 µg/mL Liberase™ and 25 µg/mL DNase solution to each tube.

Note: To ensure efficient dissociation, tubes should not be filled beyond the 30 mL mark.

Incubate the minced tissue for 45 minutes at 37° C. with continuous shaking using a Thermomixer set at 900 rpm

Pass the dissociated tissue suspension through a series of PluriSelect cell strainers (500 µm, 300 µm, 100 µm, 70 µm, 40 µm) under vacuum pressure as mentioned above and collect the flow through. Wash the strainer with 20 mL of HBSS+ buffer to collect remaining cells.

Note: Since proximal tissue is cartilaginous and bulky as compared to the distal tissue, there is a higher possibility of clogging of the filters. Using a funnel as shown in the video can help prevent overflowing of the liquid while passing through the strainers.

Add an equal volume of HBSS+ buffer to the filtrate to inhibit Liberase activity and prevent over-digestion.

Add the isolated luminal proximal airway cells from 3.1.4 to the cell suspension at this step.

Centrifuge the combined cell suspension at 600 × g for 10 mins. Remove supernatant and repeat cell wash in HBSS+ buffer.

Perform depletion of CD45⁺ immune cells and CD31⁺ endothelial cells as mentioned above in 2.4

### Cell Surface Staining for Flow Cytometry

Methods for staining proximal airway cells are as described for distal lung tissue. Viable epithelial cells are enriched based upon their CD45-negative, CD31-negative, CD236-positive cell surface phenotype and negative staining for DAPI. This epithelial cell fraction can be further subsetted based upon staining for cell type-specific surface markers, such as NGFR, allowing enrichment of basal (NGFR-positive) and non-basal (NGFR-negative; secretory, ciliated, neuroendocrine) cell types.

### Organoid Culture (Table 5)

Add 2000-5000 sorted proximal or distal epithelial cells and 7.5 × 10⁴ MRC5 cells to a sterile 1.5 mL tube.

Centrifuge at 500 × g for 5 mins at 4° C.

Note: It is important to manually confirm the cell count obtained from the sorter in order to ensure accuracy organoid colony forming efficiency.

Carefully remove and discard the supernatant and resuspend the cell pellet in 50 µl of ice-cold media supplemented with antibiotics. Keep the cell suspension on ice.

Add 50 µl of ice cold 1X growth factor depleted MatriGel® to the vial and gently pipette the suspension on ice to mix.

Note: It is important to use ice cold media and maintain cells on ice to avoid premature polymerization of the MatriGel®.

Transfer the cell suspension to a 24 well TransWell culture insert, taking care to avoid introduction of air bubbles.

Incubate at 37° C. for 30-45 mins to allow the MatriGel® to solidify.

Add 600 µl of pre-warmed growth medium to the Well.

Culture at 37° C. in a 5% CO² incubator for 30 days, during which time the media should be changed every 48 hrs.

Note: The culture duration can be altered based on the purpose of the experiment. Longer endpoints are used to study differentiation whereas shorter endpoints of 7 days, 14 days etc., can be used if the purpose of the experiment is not to achieve complete differentiation.

Note: Media was supplemented with Fungizone (0.4%) and Penstrep (1%) for the first 24 hours after seeding and 10 µM Rho kinase inhibitor (Table 4) for the first 72 hrs.

10 µM TGFβ inhibitor (Table 4) was added to the culture media for 15 days to maintain the cells in the proliferative phase and suppress overgrowth of fibroblasts.

Note: Results differ according to culture medium. Results shown herein were generated using PneumaCult™-ALI Medium, which in the Inventors’ hands results in generation of large organoids from distal lung, well differentiated and larger organoids from proximal lung.

### Organoid Staining (Table 6)

### Fixing and Embedding of Organoids

Aspirate the media from the Insert and well of TransWell and rinse once with warm PBS.

Fix the cultures by placing 300 µl PFA (2% w/v) in the Insert and 500 µl in the well for 1 hr at 37° C. Remove fixative and rinse with warm PBS taking care not to dislodge the MatriGel® plug.

Note: Fixed organoids can be stored submerged in PBS at 4° C. for one to two weeks before initiating further steps.

Aspirate PBS, invert TransWell and carefully cut TransWell membrane along its periphery. Using forceps, remove transwell membrane, taking care not to disturb the MatriGel® plug.

In a petri-dish, tap TransWell to recover the MatriGel® plug.

Add a drop of 37° C. HistoGel® to the MatriGel® plug and maintain at 4° C. until the HistoGel® solidifies.

Transfer the HistoGel®/MatriGel® plug to an embedding cassette, dehydrate through increasing concentrations of ethanol (70, 90 and 100%), clear in xylene and embed in paraffin wax.

Cut 7 µm sections on a microtome and collect on positively charged slides.

### Immunofluorescence Staining of Organoids

Place slides at 65° C. for 30 minutes to dewax the slides.

Deparaffinize the sections by immersion in xylene and rehydrate through decreasing concentrations of ethanol.

Perform High temperature antigen retrieval in Antigen Unmasking Solution, citric acid base using a Retriever 2100.

Surround the tissue with a hydrophobic barrier using a Pap pen.

Block non-specific staining between the primary antibodies and the tissue, by incubating in Blocking buffer (Table 7).

Incubate sections in the appropriate concentration of primary antibodies (Table 6) diluted in incubation solution (Table 7) overnight at 4° C. in a humidified chamber.

Rinse sections 3 times at room temperature with a washing buffer (Table 7).

Incubate in the appropriate concentration of fluorochrome conjugated secondary antibody for 1 hr at room temperature.

Rinse sections 3 times at room temperature with 0.1% Tween 20- TBS.

Incubate sections for 5 mins in DAPI (1 µg/mL).

Rinse sections once in 0.1% Tween 20- TBS., dry and mount in Fluormount G solution.

Note: Source and optimal working dilution of primary and secondary antibodies used for immunofluorescence staining are included in Table 6.

### Example 2

### Source Lung Tissue

The trachea and extrapulmonary bronchus (FIG. 1A) were used as the source tissue for isolation of proximal airway epithelial cells and subsequent generation of proximal organoids. Distal lung tissue that includes both parenchyma and small airways of less than 2 mm in diameter (FIG. 1A) were used for the isolation of small airway and alveolar epithelial cells (distal lung epithelium) and generation of either small airway or alveolar organoids. Proximal airways lined by a pseudostratified epithelium include abundant basal progenitor cells that are immunoreactive for the membrane protein NGFR (FIGS. 1B and 1C). In contrast, epithelial cells lining alveoli included a subset showing apical membrane immunoreactivity with the HTII-280 monoclonal antibody, suggestive of their alveolar type 2 cell identity (FIGS. 1B and 1D). These surface markers were used to subset single cell suspensions of epithelial cells isolated from either proximal or distal regions.

### Example 3

### Tissue Dissociation and Cell Fractionation

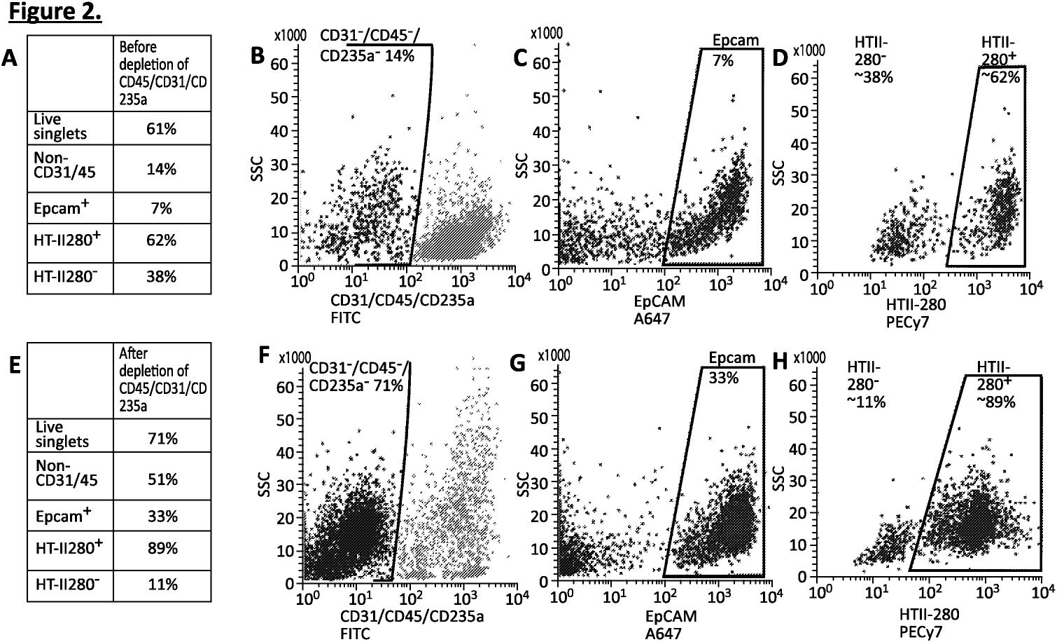

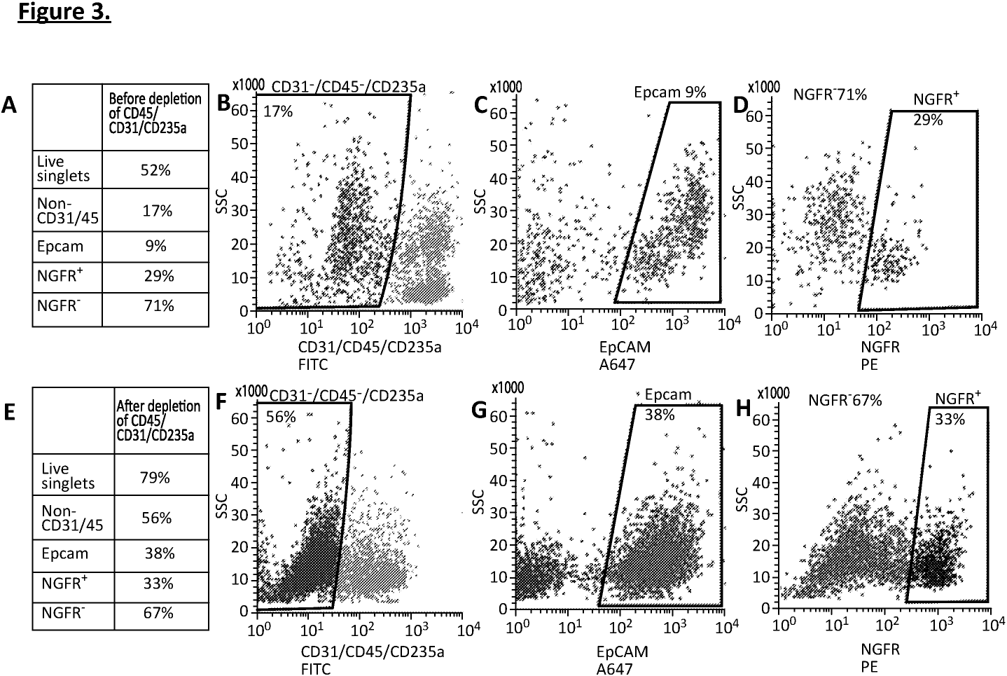

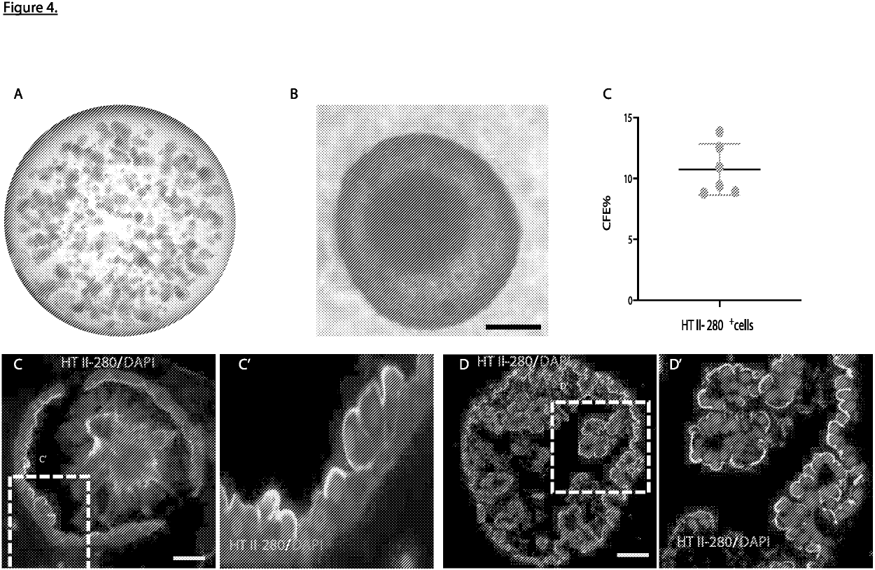

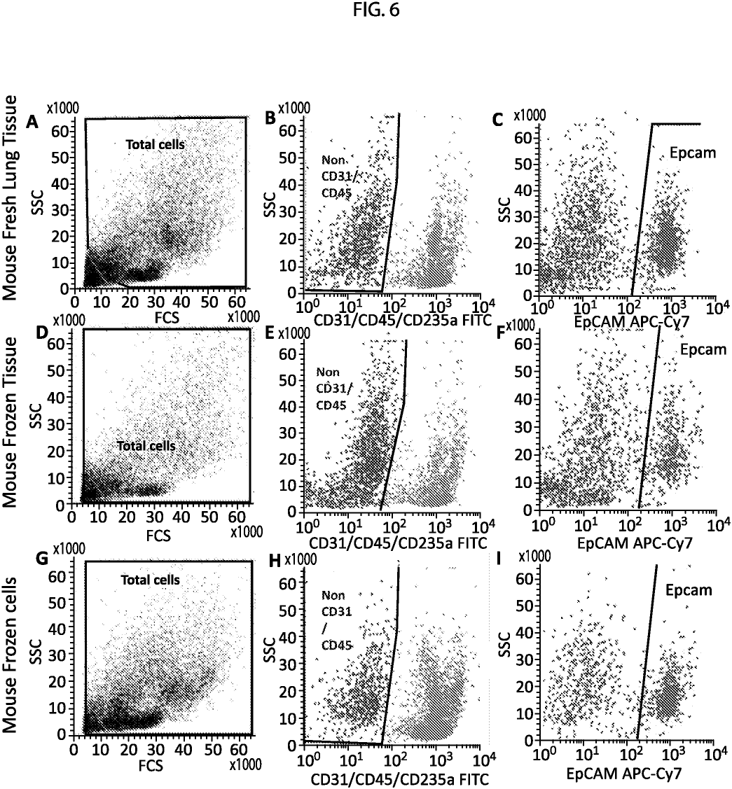

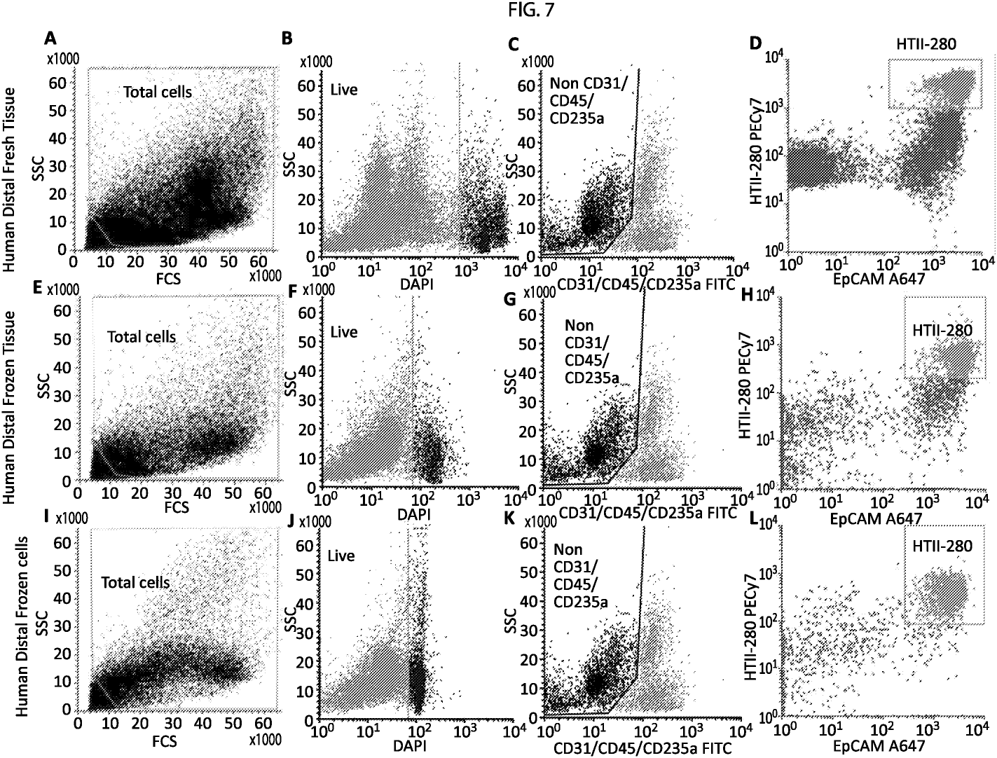

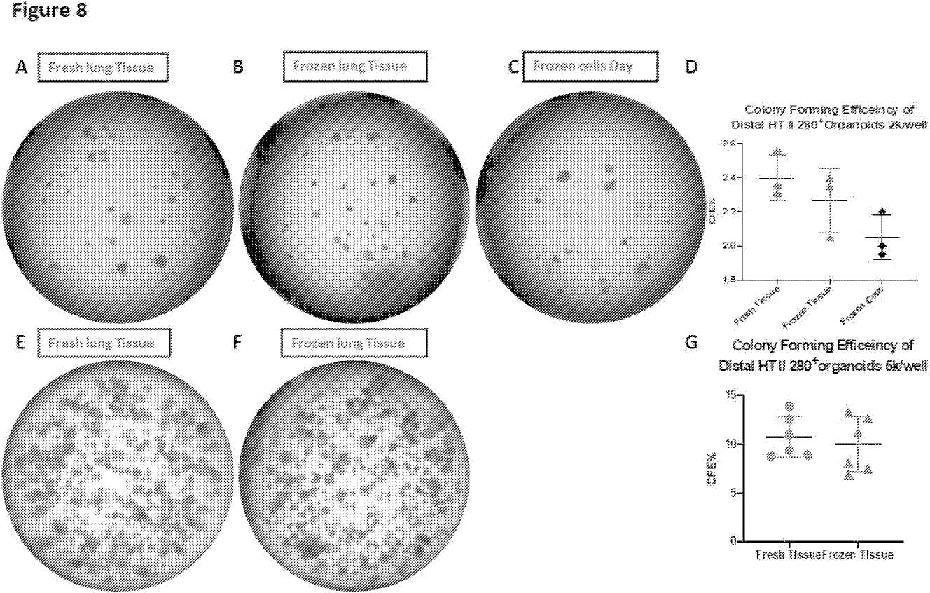

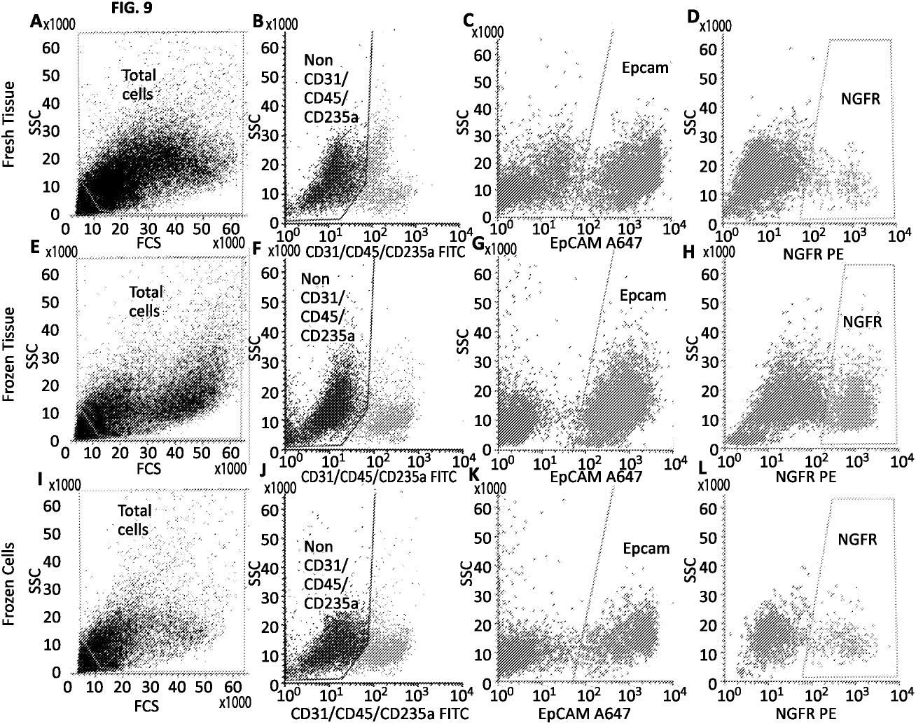

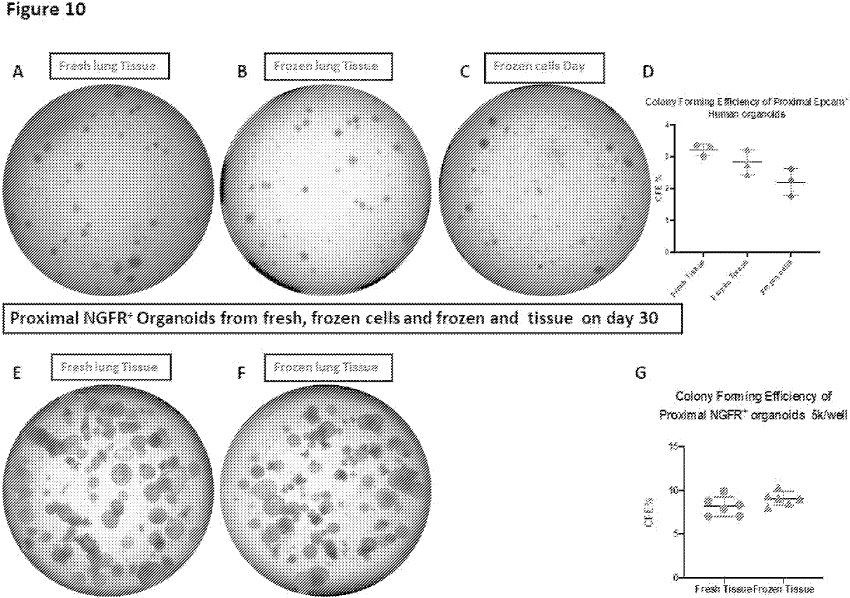

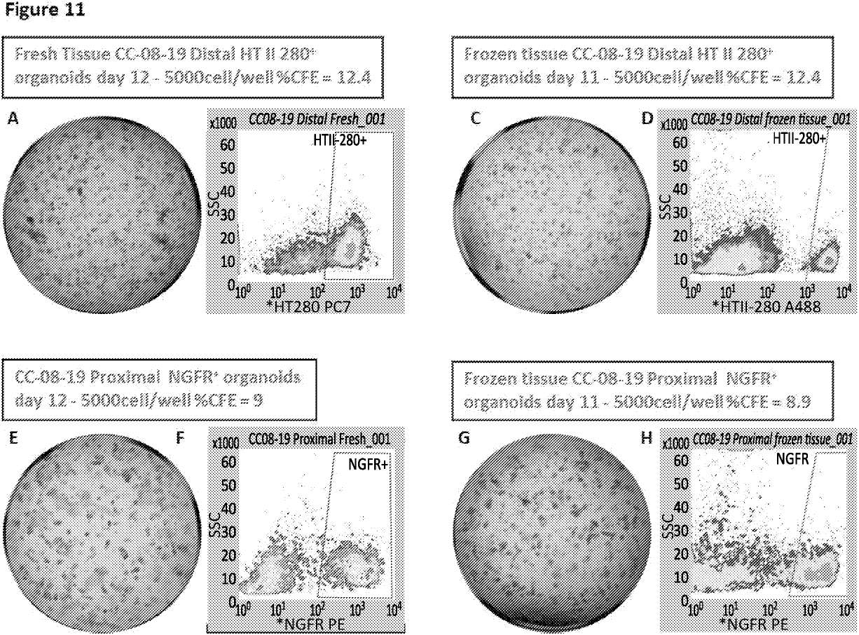

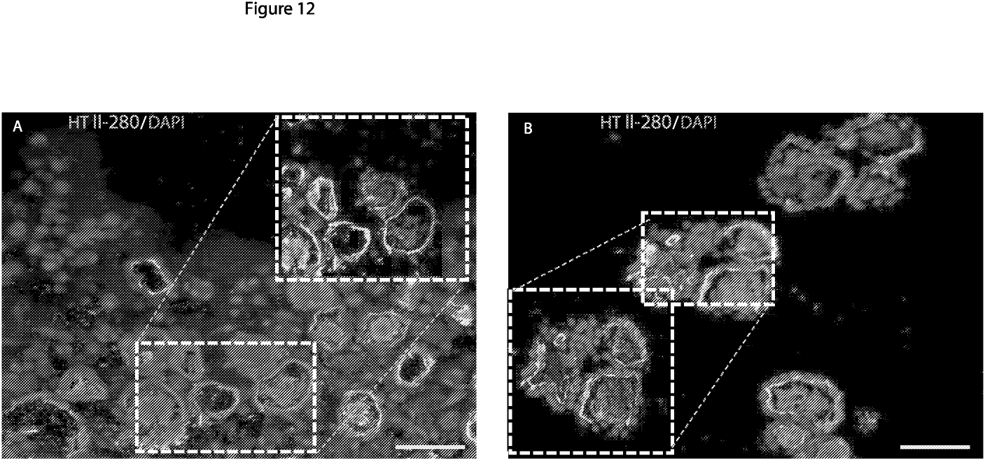

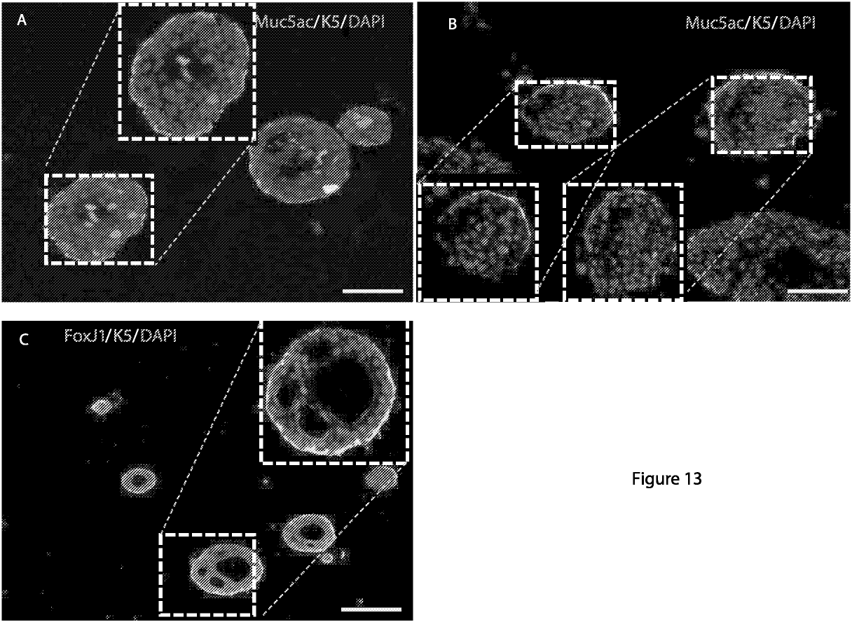

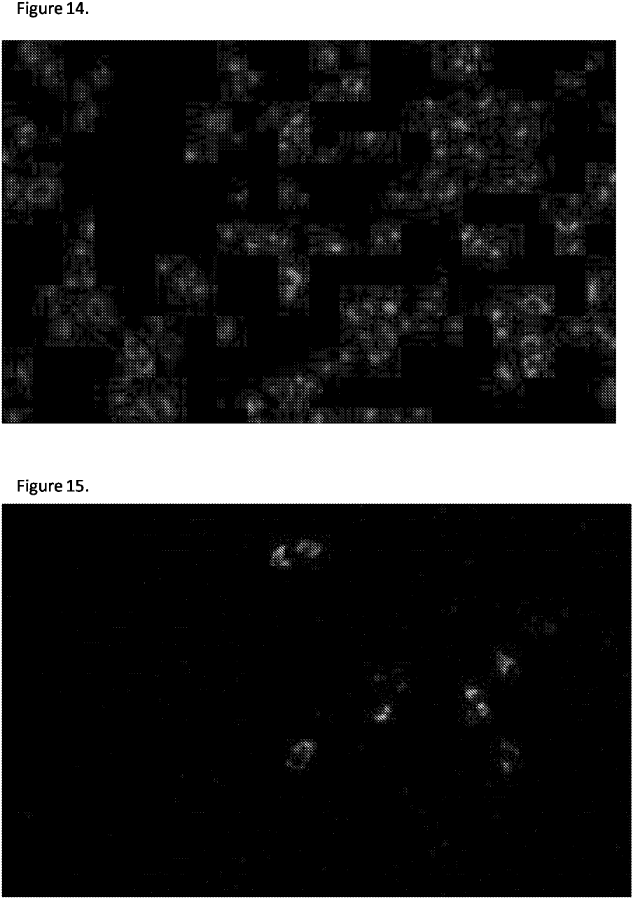

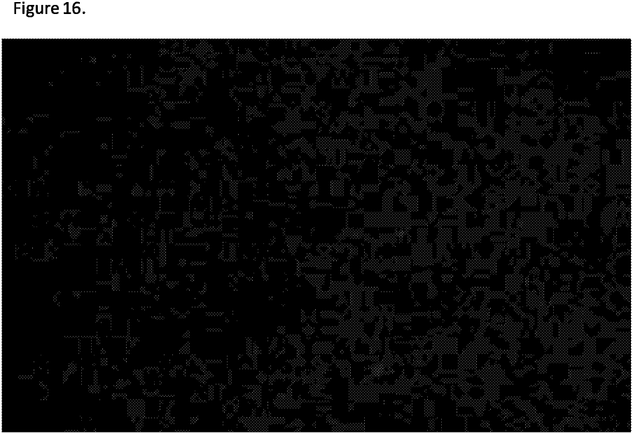

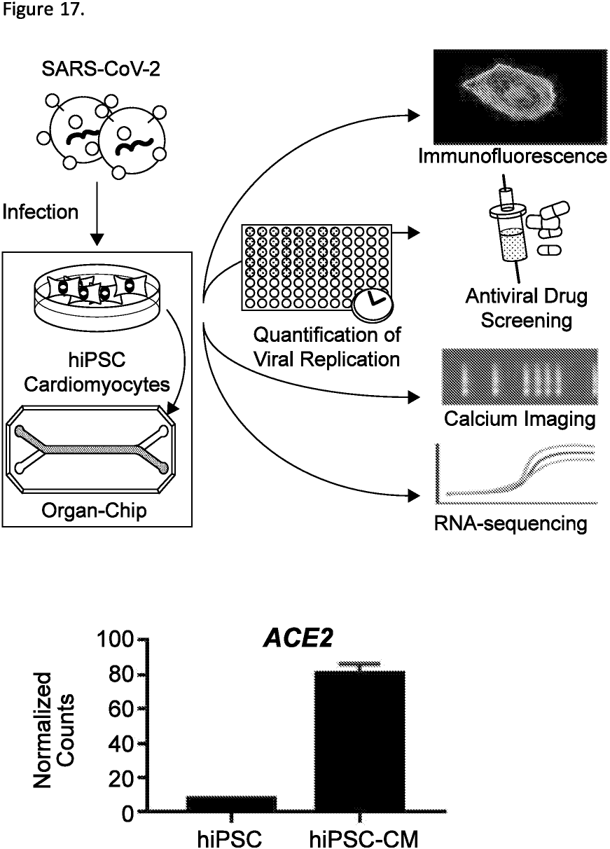

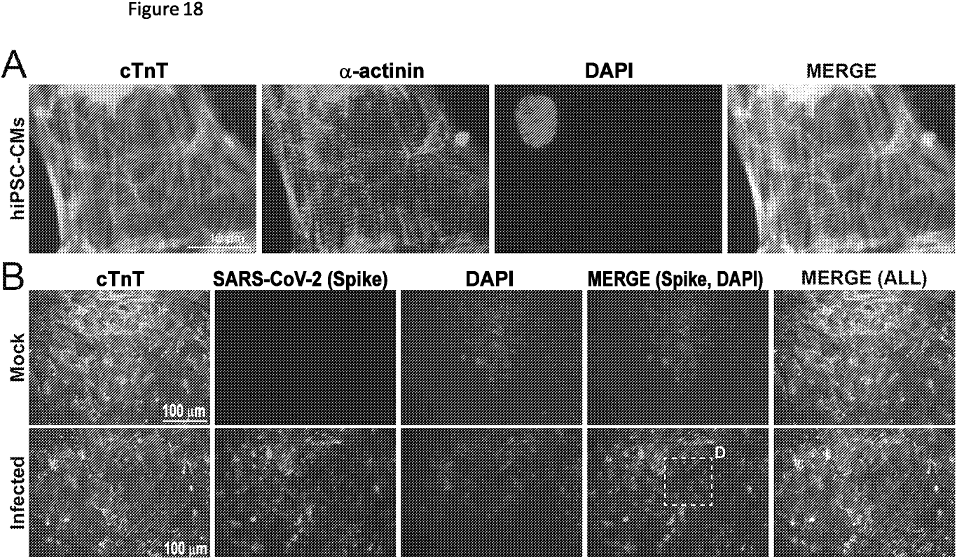

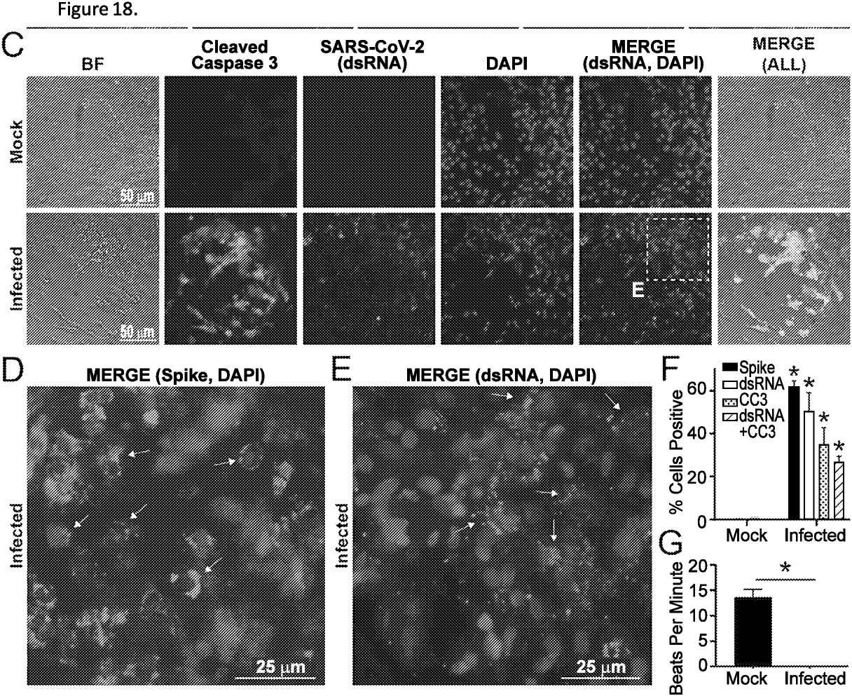

Single cell suspensions of total cells were isolated from either proximal or distal regions of human lung tissue and fractionated using both magnetic bead and FACS to yield enriched epithelial cell populations (FIG. 2 & FIG. 3). Abundant contaminating cell types including red blood cells, immune cells and endothelial cells were stained using antibodies to CD235a, CD45 and CD31, respectively, followed by magnetic-associated cell sorting (MACS) for depletion of these cell types from the total pool of lung cells. The resulting “depleted” cell suspensions were significantly enriched for epithelial cell populations in both distal (FIG. 2E) and proximal (FIG. 3E) tissue samples, with corresponding increase in FACS efficiency. After depletion of CD235al CD45/CD31 positive cells using MACS the percentage of CD32⁻/CD45⁻/CD235a⁻ increased from 14% (FIGS. 2A, 2B) to 51.7% (FIGS. 2E, 2F) in distal population. Further FACS depletion of cells staining positively for either CD235a, CD45 or CD31, elimination of cells with positive staining for DAPI and positive selection for the epithelial cell surface marker CD326, led to highly enriched distal cell population that accounted for 33.5% (FIGS. 2E and 2G) compared to 7% (FIGS. 2A, 2D) before depletion of negative population. Further subsetting of distal epithelial cell populations was achieved by fractionation based upon surface staining with the HTII-280 monoclonal antibody (FIGS. 2D & 2H), respectively. Accordingly, distal lung epithelial cells included 4.3% HTII-280⁺ and 2.6% HTII-280⁻ subsets (FIG. 2D without depleting of CD31/CD45/CD235a) and 30% HTII-280⁺ and 3.6% HTII-280⁻ subsets (FIG. 2H after depleting of CD31/CD45/CD235a).

Total cells isolated from proximal region were depleted for CD235a/ CD45/CD31 positive cells using MACS and the % of CD31⁻/CD45⁻/CD235a⁻ increased from 17% (FIGS. 3A, 3B) to 56.6% (FIGS. 3E, 3F). Positive selection for the epithelial cell surface marker CD326 in cells isolated from proximal region, led to highly enriched proximal cell population that accounted for 38% (FIGS. 3E and 3G) of total lung cell fractions compared to 9.3% (FIGS. 3A,3D) without depletion of negative population respectively. Further subsetting of proximal epithelial cell populations was achieved by fractionation based upon surface staining with antibodies to NGFR (FIGS. 3D, 3H), respectively. Accordingly, proximal lung epithelial cells included 2.7% NGFR⁺ and 6.5% NGFR⁻ subsets (FIG. 3D without depleting of CD31/CD45/CD235a) and 13% were NGFR⁺ and 25% NGFR⁻ (FIG. 3H after depleting of CD31/CD45/CD235a).

### Example 4

### Lung Organoid Cultures

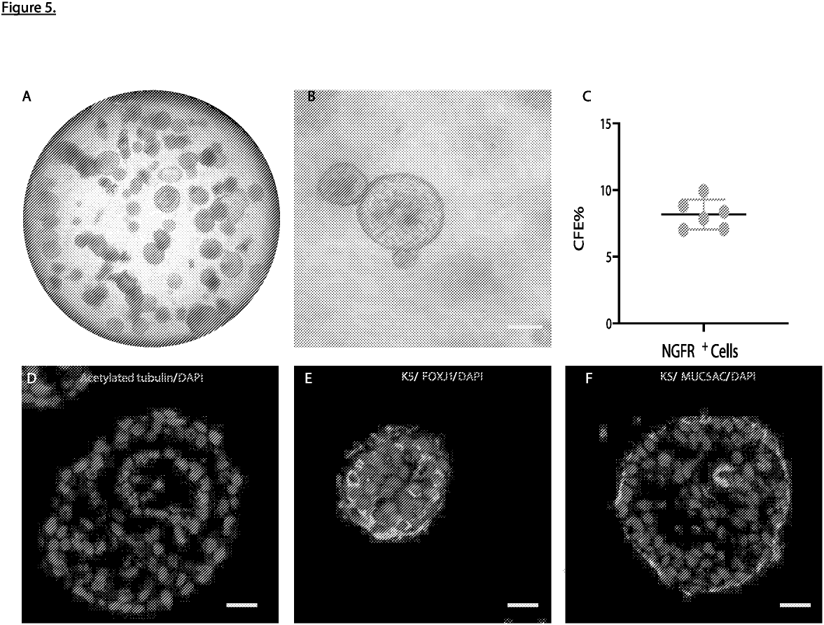

Distal lung epithelial organoids were cultured within growth-factor depleted MatriGel® in media that were empirically tested to optimize for organoid growth and differentiation. Three different media were evaluated including PneumaCult™-ALI medium, small airway epithelial cell growth medium (SAECG medium) and mouse Basal medium (media compositions are included in Table 4). Optimal organoid growth was obtained using PneumaCult™-ALI medium, which was selected for further studies. Cultures of HTII-280⁺ distal lung epithelial cells yielded rapidly expanding organoids with an average colony-forming efficiency of 10% (FIGS. 4A-4C). Immunofluorescence staining of day 30 cultures using the HTII-280 monoclonal antibody revealed lumen-containing organoids composed predominantly of HTII-280+ distal lung epithelial cells (FIGS. 4D, 4D’ and 4E, 4E’). Cultures of distal lung epithelial HTII-280- cells yielded organoids that were composed of a pseudostratified epithelium resembling that of small airways (not shown).

Proximal lung epithelial organoids were cultured from NGFR⁺ cells seeded into MatriGel® and cultured for 30 days in PneumaCult™-ALI medium. Large lumen-containing organoids were observed (FIGS. 5A and 5B) with an average colony-forming efficiency of 7.8% (FIG. 5C). Organoids were composed of a pseudostratified epithelium composed of self-renewing Krt5-immunoreactive basal cells and differentiated luminal cell types including AcT+/FOXJ1+ ciliated cells and MUC5AC+ secretory cells (FIGS. 5D-F).

### Example 5

### Materials List

TABLE 1

Materials for cell isolation
Cell Isolation

| | | |
|---|---|---|
| VWR® Razor Blades | VWR | 55411-050 |
| Falcon® Disposable Petri Dishes, Sterile, Coming® | VWR | 25373-187 |
| pluriStrainer® 40 µm (Cell Strainer) | Pluriselect | 43-50040-51 |
| pluriStrainer® 70 µm (Cell Strainer) | Pluriselect | 43-50070-51 |
| pluriStrainer® 100 µm (Cell Strainer) | Pluriselect | 43-50100-51 |
| pluriStrainer® 300 µm (Cell Strainer) | Pluriselect | 43-50300-03 |
| pluriStrainer® 300 µm (Cell Strainer) | Pluriselect | 43-500500-03 |
| Funnel | Pluriselect | 42-50000-03 |
| connecting ring | Pluriselect | 41-50000-01 |
| Red Blood Cell lysis buffer | eBioscience | 00-4333-57 |
| Liberase™ TM Research Grade | Sigma Aldrich | 5401127001 |
| HBSS Hank’s Balanced Salt Solution 1× 500 ml | VWR | 45000-456 |
| 30 mL Sterile syringes, Luer-Lok Tip | VWR | BD302832 |
| BD Precisionglide needle 16G | VWR | 305198 |
| BD Precisionglide needle 18G | VWR | 305199 |
| Red Biosafety Bags | | |

TABLE 2

Composition of the FACS Buffer

| | | | |
|---|---|---|---|
| HBSS Hank’s Balanced Salt Solution 1× 500 ml | VWR | 45000-456 | 500 ml bottle |
| EDTA (0.5 M), pH 8.0, RNase-free | Thermofisher scientific | AM9260G | 500 µl |
| HEPES (1 M) | Thermofisher scientific | 15630080 | 5 ml |
| Amphotericin B | Thermofisher scientific | 15290018 | 2 ml |
| Penicillin-Streptomycin-Neomycin (PSN) Antibiotic Mixture | Thermofisher scientific | 15640055 | 5 ml |
| Fetal Bovine Serum | Gemini Bio-Products | 100-106 | 10 ml |

TABLE 3

Equipment

| | | |
|---|---|---|
| Thermomixer | Eppendorf | 05-412-503 |
| GentleMACS Octo Dissociator | MACS Miltenyi Biotec | 130-095-937 |
| GentleMACS C Tubes | MACS Miltenyi Biotec | 130-096-334 |
| LS Columns | MACS Miltenyi Biotec | 130-042-401 |
| Leica ASP 300 s Tissue processor | | |

TABLE 4

Composition of Organoid Culture mediums

| ThinCert™ Tissue Culture Inserts, Sterile | Greiner Bio-One | 662641 | |
|---|---|---|---|
| PneumaCult™-ALI Medium | StemcellTechnologies | 5001 | |
| Small Airway Epithelial cell Growth Medium | PromoCell | C-21170 | |
| Y-27632 (ROCK inhibitor) 100 mM stock (1000×) | Stemcell Technologies | 72302 | |
| Mouse Basal medium | | | |
| DMEM/F-12, HEPES | ThermoFisher scientific | 11330032 | 50 ml |
| Insulin-Transferrin-Selenium (ITS -G) (100×) | ThermoFisher scientific | 41400045 | 500 µl |
| Amphotericin B | Thermofisher scientific | 15290018 | 50 µl |
| Penicillin-Streptomycin-Neomycin (PSN) Antibiotic Mixture | Thermofisher scientific | 15640055 | 500 µl |
| Fetal Bovine Serum | Gemini Bio-Products | 100-106 | 5 ml |
| SB431542 TGF-β pathway inhibitor (stock 10 mM) | Stem cell | 72234 | 5 µl |

TABLE 5

List of antibodies for FACS

| | | | |
|---|---|---|---|
| Mouse igM anti human HT2-280 | Terrace Biotech | TB-27AHT2-280 | 1:300 |
| FITC anti-human CD31 | BioLegend | 303104 | 1:100 |
| FITC anti-human CD45 | BioLegend | 304054 | 1:100 |
| FITC anti-human CD235a | BioLegend | 349104 | 1:100 |
| Alexa Fluor® 647 anti-human CD326 (EpCAM) Antibody | BioLegend | 369820 | 1:50 |
| PE anti-human CD271(NGFR) | BioLegend | 345106 | 1:50 |
| CD31 MicroBead Kit, human | Miltenyi Biotec | 130-091-935 | 20 µl/ 10⁷ total cells |
| CD45 MicroBeads, human | Miltenyi Biotec | 130-045-801 | 20 µl/ 10⁷ total cells |
| DAPI | Sigma Aldrich | D9542-10MG | 1:10000 |

TABLE 6

List of antibodies for immunofluorescence

| Histogel | Thermo Scientific | HG-4000-012 | |
|---|---|---|---|
| Primary Antibodies | | | |
| Anti HT2-280 | Terracebiotech | TB-27AHT2-280 | 1:500 |
| Keratin 5 Polyclonal Chicken Antibody, Purified [Poly9059] 100 µl | Biolegend | 905901 | 1:500 |
| PDPN / Podoplanin Antibody (clone 8.1.1) | LifeSpan Biosciences | LS-C143022-100 | 1:300 |
| MUC5AC Monoclonal Antibody (45M1) | ThermoFisher Scientific | MA5-12178 | 1:300 |
| Sox-2 Antibody | Santa Cruz biotechnologies | sc-365964 | 1:300 |
| FOXJ1 Monoclonal Antibody (2A5) | Thermo Fisher Scientific | 14-9965-82 | 1:300 |
| Purified Mouse Anti-E-Cadherin | bd biosciences | 610182 | 1:1000 |
| Human Uteroglobin/SCGB1A1 Antibody | R and D systems | MAB4218 | 1:300 |
| Secondary Antibodies | | | |
| FITC anti-mouse IgM Antibody | BioLegend | 406506 | 1:500 |
| Goat anti-Hamster IgG (H+L), Alexa Fluor 594 | Thermo Fisher Scientific | A-21113 | 1:500 |
| Goat anti-Mouse IgG2a Cross-Adsorbed Secondary Antibody, Alexa Fluor 488 | Thermo Fisher Scientific | A-21131 | 1:500 |
| Goat anti-Mouse IgG2a Cross-Adsorbed Secondary Antibody, Alexa Fluor 568 | Thermo Fisher Scientific | A-21134 | 1:500 |
| Goat anti-Mouse IgG2b Cross-Adsorbed Secondary Antibody, Alexa Fluor 568 | Thermo Fisher Scientific | A-21144 | 1:500 |
| Donkey anti-rabbit 1gG, 488 | Thermo Fisher Scientific | A-21206 | 1:500 |
| Goat anti-Mouse IgG 1 Cross-Adsorbed Secondary Antibody, Alexa Fluor 488 | Thermo Fisher Scientific | A-21121 | 1:500 |

### Example 6

### Advantages of Cryobanked Human Lung Tissue

Frozen lung tissue provides better logistical flexibility and can be shipped like frozen cells for analysis at different sites.

Lung samples collected from different patients at different time points can be cryobanked for simultaneous processing for either genomic or functional analysis.

Viability of cells isolated following application of this cryobanking protocol is comparable that seen with fresh tissue.

### Example 7

### Protocols

Human lung tissue was obtained from deceased tissue donors in compliance with consent procedures developed by International Institute for the Advancement of Medicine (IIAM) and approved by the Cedars-Sinai Medical Center Internal Review Board.

### Freezing Distal and Proximal Regions of the Lung

Prepare and autoclave all dissection instruments, glassware and the appropriate solutions one day prior to cell isolation.

Upon receiving lung tissue identify and separate the proximal and distal regions and mince the tissue as described in Konda et al 2020 (refer the first article here). Briefly, dice tissue into approximately 1 cm3 pieces and place in a clean 50 mL tube. Wash tissue three times with chilled HBSS, discarding the HBSS wash each time to remove blood and epithelial lining fluid.

Place tissue in a new petri dish (150 × 15 mm) and blot dry with sterile Kim wipes. Using forceps and scissors, remove as much visceral pleura as possible. Use scissors to mince tissue into pieces of approximately 3-4 mm diameter.

Add approximately 1 to 1.5 grams of tissue to a 2 mL cryovial and add 1 mL of the cryoprotective media, CryoStor (Table 1).

Label tubes and move the vials to a cell freezing container. Fill the freezing container with isopropyl alcohol place them in -80 degrees overnight.

Transfer the frozen vials to the vapor phase of a liquid nitrogen vessel and record the locations.

Note: Ensure that the tissue is completely submerged in freezing medium in order to prevent formation of ice crystals during the freezing process and prevent tissue damage.

### Enrichment and Sub Setting of Epithelial Progenitor Cells From Fresh Lung Tissue

Enrichment and fractionation of the epithelial, small airway and alveolar epithelial progenitor cells from distal lung tissue or tracheobronchial basal progenitor cells from proximal lung tissue using appropriate cell surface markers as described in Konda et al 2020

### Enrichment and Sub Setting of Epithelial Progenitor Cells From Frozen Lung Tissue

Thaw frozen proximal or distal tissue by placing the vial at 37 degrees for 1-2 mins.

Transfer the tissue to a sterile Petri dish, add 5 ml of FACS buffer (Table 1) to the tissue and allow the tissue to equilibrate in an incubator at 37° C. for 10 mins.

Transfer the contents into a sterile 50 ml tube and centrifuge at 500 × g for 5 mins.

Aspirate the supernatant and move the tissue to a new sterile Petri dish. Finely mince the tissue using single sided razor blade and perform enzymatic digestion to obtain a single cell suspension of total cells as mentioned in Konda et al 2020.

Deplete CD31+ endothelial cells and CD45+ immune cells from the pool of total cells using the Milteny MACS CD31 & CD45 microbeads conjugated to monoclonal anti-human CD31 and CD45 antibody (isotype mouse IgG1) and LS columns in accordance to the manufacturer’s protocol.

Collect flow through consisting primarily of epithelial and stromal cells in a fresh sterile tube and centrifuge it at 600 × g for 5 mins at 4° C. Perform a cell count to ascertain the total number of cells in the flow through

### Cell Surface Staining for Fluorescence Associated Cell Sorting (FACS) in Both Proximal and Distal Lung

Resuspend 1 × 10⁷ cells per 1 mL of HBSS+ buffer. Add primary antibodies at the required concentration and incubate the cells for 30 mins at 4° C. in dark. Details of antibody sources and titers are described in Konda et al 2020.

Wash off excess antibody by adding 3 mL of HBSS+ buffer and centrifuge at 600 × g for 5 mins at 4° C.

Discard supernatant and resuspend cells in HBSS+ buffer per 1 × 10⁷ cells/ mL.

Filter cells into 5 mL polystyrene tubes through a strainer cap to ensure formation of a single cell suspension.

Add DAPI (1 µg/mL) to stain permeable (dead) cells.

### Organoid Culture and Staining

The protocol for organoid culture and staining is explained in detail in Konda et al 2020. Mention how many cells you seed per transwell.

### 10x RNA Sequencing Experiment

Capture the CD45-CD31-CD326+ lung epithelial cells from fresh and frozen tissue using a 10X Chromium device (10X Genomics) and prepare libraries according to the Single Cell 3′ v2 Reagent Kits User Guide (10X Genomics).

Quantify the barcoded sequencing libraries by quantitative PCR using the KAPA Library Quantification Kit (KAPA Biosystems, Wilmington, MA).

Sequenced the libraries using Novaseq 6000 (Illumina)

### Example 8

### Induced Pluripotent Stem Cell-Derived Cardiomyocytes (iPSCs-CMs)

### Cardiac Differentiation of Human iPSC

NOTE: All media should be at least at RT when added.

After dissociation with EDTA or 1x cell detachment solution, seed approximately 100,000 human iPSCs on ECMS coated 6-well culture plates for differentiation (same steps as cell passaging procedures). When the cells reach 85% confluency, change the medium to RPMI/B27 without insulin medium with 6 µM GSK3-beta inhibitor CHIR99021 (CHIR) and maintain for 48 hr.

After 48 hr, replace the CHIR-containing culture medium with RPMI/B27 without insulin medium and leave alone for 24 hr (until day 3).

At day 3, change the media to RPMI/B27 without insulin with 5 µM Wnt inhibitor IWR1 and maintain for 48 hr (until day 5). NOTE: Wnt inhibition can also be attempted using other small molecule compounds, as described in earlier studies¹¹. IWR1 was selected over other small molecule Wnt inhibitors due to the increased range in which it has been shown to be effective in inhibiting Wnt signaling¹¹.

At day 5, change the medium back to RPMI/B27 without insulin medium and leave for 48 hr (until day 7).

At day 7, replace the medium with RPMI/B27 medium (with insulin) and replace medium every 3 days thereafter with the same medium. Spontaneous beating of cardiomyocytes should first be visible at approximately day 8 to day 10.

### Purification of Human Cardiomyocytes Through Glucose Starvation

At day 10 post-differentiation, change the medium in each well of the 6-well plate to 2 ml low glucose medium and maintain the cells in this medium for 3 days (until day 13).

At day 13, return cells to RPMI/B27 medium (with insulin).

Optionally, replate cardiomyocytes prior to the second round of glucose starvation to help loosen non-cardiomyocytes from the culture plate, allowing for easier dissociation of non-cardiomyocytes during glucose starvation.

At day 13, aspirate medium, wash once with PBS, and dissociate the cells into single cells using 500 µl of cell disassociation enzyme for 5 min at 37° C. Specifically, after 5 min of enzyme treatment, use a 1,000 µl pipette to manually dissociate cardiomyocytes from the 6-well plate by repeatedly pulling up the cell disassociation enzyme and spraying it against the cardiomyocyte monolayer. Up to 30 pipetting repetitions may be required to dissociate the cardiomyocytes into single cells.

After cells are dissociated and are in single-cell form, collect all cells into a 15 ml conical tube filled with 5 ml of RPMI/B27 medium with insulin to dilute out the cell disassociation enzyme and centrifuge for 4 min at 200 × g. Aspirate and discard the supernatant.

Re-suspend the cells with 2 ml RPMI/B27 medium and plate onto a new ECMS-coated 6-well plate. Typically, higher confluency of cardiomyocytes helps with cell survival during replating. Aim to replate 2 million cells per new 6-well dish for optimal survival during replating.

At day 14, change the medium back to 2 ml of low glucose medium for a second glucose deprivation cycle. Culture the cells in this low glucose state for 3 more days. Most of the non-cardiomyocytes will die in this low-glucose culture condition.

At day 17, change the medium to 2 ml of RPMI/B27 medium with insulin. The remaining cells will be highly purified cardiomyocytes. These cardiomyocytes can be used for gene expression analysis, drug screening, metabolic analysis, and various other downstream assays.

Further information is found in, Sharma, A., Li, G., Rajarajan, K., Hamaguchi, R., Burridge, P.W., and Wu, S.M. (2015). Derivation of highly purified cardiomyocytes from human induced pluripotent stem cells using small molecule-modulated differentiation and subsequent glucose starvation. Journal of visualized experiments: JoVE., which is fully incorporated by reference herein.

### Example 9

### Induced Pluripotent Stem Cell-Derived Endothelial Cells (iPSCs-ECs)

Techniques for producing induced pluripotent stem cell-derived endothelial cells include:

In one example, iECs can be made by culturing (iPSCs) in the presence of CHIR99012 for about 2 days to generate mesoderm, culturing mesoderm in the presence of BMP4, VEGF, and FGF2 for about 2 days to generate vascular progenitor cells, culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In various embodiments, the vascular progenitors are cultured in the presence of EGM-MV3 and VEGF, and passages 2, 3, 4 or more times to generate endothelial cells. For example, iECs can express key markers such as CD31, CD34, VEGF, and VEGFA. Using a combination of growth factors, the Inventors were able to successfully produce endothelial cell types. Based on the described protocols, it appears that endothelial markers are more and purely expressed in Day 20 compared to Day 10 of differentiation -> time for maturation. Differentiation to be confirmed with other experiments: Dil-ac-LDL uptake, and TEER (resistance).

Alternatively, one can generate a quantity of endothelial cells made by a method of generating endothelial cells, including culturing (iPSCs) in the presence of CHIR99012 for about 2 days to generate mesoderm, culturing mesoderm in the presence of BMP4, VEGF, and FGF2 for about 2 days+ to generate vascular progenitor cells, culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In other embodiments, the vascular progenitor cells express one or more of: CD31+, CD34+, VEGF+, and VEGFA+ at day 20.

Methods to convert human induced pluripotent stem cells (hiPSCs) to cardiomyocytes (hiPSC-CMs) and endothelial cells (hiPSC-ECs) have enabled cardiovascular cells to be mass-produced in vitro for disease modeling and drug screening. The hiPSC-CMs express most ion channels and sarcomeric proteins found in adult human CMs and can spontaneously contract. Similarly, hiPSC-ECs can uptake LDL and undergo angiogenic sprouting. These hiPSC-CMs and hiPSC-ECs can be made in approximately two weeks using defined differentiation protocols, and they can be genetically customized using genome editing technologies such as CRISPR/Cas9. Additionally, hiPSC-derived CMs and ECs can recapitulate, at the cellular level, phenotypes for cardiovascular diseases including viral myocarditis, Brugada syndrome, long-QT syndrome, dilated cardiomyopathy, hypertrophic cardiomyopathy, chemotherapy-induced cardiomyopathy, and congenital heart disease. The hiPSC-CMs are also responsive to inotropic drugs such as norepinephrine, and their beating rates can be controlled via electrical stimulation. Because hiPSC-CMs and hiPSC-ECs can be purified and replated for downstream applications, research groups in academia and industry have started utilizing these cells as a complementary platform to non-human and non-cardiovascular model systems, for high-content imaging and drug screening assays.

### Example 10

### Human iPSCs and Organ Chips Model SARS-CoV-2-Induced Viral Myocarditis

Coronavirus disease 2019 (COVID-19), which is caused by the severe acute respiratory syndrome coronavirus 2 (SARS-CoV-2). There is mounting evidence that SARS-CoV-2 infection may cause cardiac complications including elevated cardiac stress biomarkers, arrhythmias, and heart failure. A recent Chinese study demonstrated significantly elevated troponin levels among some COVID-19 patients, indicating cardiac injury, and notably, cardiac injury was associated with increased risk of mortality.

The etiology of cardiac injury in COVID-19, however, remains unclear. Studies have raised suspicion for cardiac injury mediated by direct myocardial viral infection and resulting myocarditis. Cardiac tissue highly expresses the ACE2 receptor, further suggesting feasibility of direct viral internalization in cardiomyocytes. Alternatively, cardiac injury may be ischemia-mediated. The profound inflammatory and hemodynamic impacts seen in COVID-19 has been hypothesized to cause atherosclerotic plaque rupture or oxygen supply-demand mismatch resulting in ischemia. Elucidating the mechanism of cardiac injury will be essential in guiding therapeutic strategies. Specifically, antiviral agents could potentially mitigate cardiac complications if the underlying mechanism of cardiac injury is direct viral infection. The aforementioned platform allows evaluation of whether SARS-CoV-2 can directly infect human cardiomyocytes, study the behavior of virally infected cardiomyocytes, and establish a cardiomyocyte-specific antiviral drug screening platform against SARS-CoV-2.

### Example 11

### Modeling SARS-CoV-2 Acute Viral Infection Using hiPSC-Derived Cardiomyocytes

As described, the hiPSC-CMs express relevant proteins found in adult human CMs, can spontaneously contract, can be made in weeks using defined differentiation protocols, and can be genetically customized using genome editing. Additionally, hiPSC-derived CMs can recapitulate cellular phenotypes for cardiovascular diseases including viral myocarditis, hypertrophic cardiomyopathy, and chemotherapy-induced cardiomyopathy.

The hiPSC-CMs are responsive to inotropic drugs such as norepinephrine, and beating rates can be controlled via electrical stimulation. Because hiPSC-CMs can be purified and replated for downstream applications, research groups in academia and industry have started utilizing these cells as a complementary platform to non-human and non-cardiovascular model systems, for high-content imaging and drug screening assays. hiPSC-CMs can serve as an in vitro model for coxsackievirus B3-induced myocarditis and antiviral drug screening platform. This study showed that hiPSC-CMs express the receptor needed for infection by coxsackievirus (another positive-sense RNA virus), are susceptible to viral infection, and can be used to predict antiviral drug efficacy.

The Inventors propose adapting the methodologies developed in this previous study, along with integrating microfluidic cardiac organ-chip technology, to establish a hiPSC-CM-based, in vitro model of SARS-CoV-2 cardiac-specific viral infection. This in vitro, mechanistic study using human induced pluripotent stem cell-derived cardiomyocytes (hiPSC-CMs) can serve as a surrogate model system for true human cardiomyocytes. The Inventors will use hiPSC-CMs derived from multiple hiPSC lines representative of a diverse range of individuals, since all populations are susceptible to infection by SARS-CoV-2.

### Example 12

### Determine hiPSC-CM Susceptibility and Functional Response to Infection by SARS-CoV-2

First, hiPSC-CMs will be produced from 3 independent hiPSC lines Preliminary data indicates that hiPSC-CMs express the ACE2 receptor required for SARS-CoV-2 viral internalization, and thus hiPSC-CMs are susceptible to infection. One introduces SARS-CoV-2 onto the hiPSC-CMs at a high multiplicity of infection, to ensure viral internalization. Given that the known replication time for coronavirus is hours, The Inventors anticipate expression of SARS-CoV-2 proteins to occur within hours of infection on hiPSC-CMs. The Inventors will conduct immunofluorescence for viral proteins such as the SARS-CoV-2 “spike” glycoproteins to confirm infection, similar to immunofluorescence for coxsackievirus viral protein 1 (VP1) in The Inventors’ previous study. Additionally, since viral infection is known to induce cardiac arrhythmias, The Inventors will use phase contrast microscopy and calcium imaging to examine SARS-CoV-2-induced changes in hiPSC-CM calcium handling and beating patterns. As shown previously, hiPSC-CMs may experience alterations in contractility prior to the onset of visible virus-induced cytopathic effect. Thus, calcium imaging will be conducted within hours after initial infection. This will allow confirmation of whether human cardiomyocytes are susceptible to acute viral infection by SARS-CoV-2. Such results could provide the clinicians responsible for managing COVID-19 patients with critical information about the cardiac-specific effects of COVID-19, and may lead to the development of novel treatment strategies.

In a preliminary study, infection of purified hiPSC-CMs by SARS-CoV-2 and functional contractility analysis, purified hiPSC-CMs were replated into 96-well plates at 100,000 cells per well and allowed to regain contractility (Supplemental Movie 1) before being subjected to SARS-CoV-2 infection. The SARS-CoV-2 was obtained from the Biodefense and Emerging Infections (BEI) Resources of the National Institute of Allergy and Infectious Diseases (NIAID)and titered on Vero-E6 cells (see Methods). The hiPSC-CMs were infected with SARS-CoV-2 at a multiplicity of infection (MOI) of 0.1 for 72 hours. A mock treatment without virus was used as a control condition. After 72 hours, hiPSC-CMs from both mock and infected conditions were fixed in 4% paraformaldehyde and stained for cardiac marker cardiac troponin T (cTnT), SARS-CoV-2 viral capsid “spike” protein, and DAPI nuclear stain to determine if the virus can enter and proliferate within hiPSC-CMs (FIG. 1B). The infected hiPSC-CMs stained positively for spike protein,suggesting that SARS-CoV-2 can establish active infection in hiPSC-CMs. To determine if SARS-CoV-2 induced a cytopathic effect on hiPSC-CMs, mock and infected hiPSC-CMs were stained for the apoptosis marker cleaved caspase-3, as well as for the double stranded RNA (dsRNA) intermediate unique to positive sense RNA virus infection (FIG. 1C). The dsRNA and spike protein stains represent two independent assays for visualizing SARS-CoV-2 viral uptake and genome replication in hiPSC-CMs. A proportion of infected cells were positive for dsRNA and also stained positive for cleaved caspase-3, indicating that hiPSC-CMs were undergoing virus-induced apoptosis. Notably, both the SARS-CoV-2 spike protein and dsRNA intermediates localized at perinuclear regions in hiPSC-CMs (FIGS. 1D, 1E), consistent with prior results with coronavirus infection in non-cardiomyocytes (Hagemeijer et al., 2012) and coxsackievirus infection on hiPSC-CMs (Sharma et al., 2014). This suggests the presence of a viroplasm whereby hiPSC-CM ribosomal machinery and other membranous components are being co-opted for viral replication and protein translation. Immunofluorescence results were quantified, indicating percentages of cells that were positive for viral stains against dsRNA and spike protein, as well as cells positive for cleaved caspase-3 (FIG. 1F). Simple beat rate contractility analysis was also conducted, whereby 30 second videos were taken of wells containing mock and infected hiPSC-CMs at 72 hours after infection with SARS-CoV-2 (Supplemental Movie 1). Functionally, infected hiPSC-CMs ceased beating after 72 hours of SARS-CoV-2 infection, whereas mock wells continued to contract (FIG. 1G). Taken together, these results suggest that hiPSC-CMs are susceptible to SARS-CoV-2 infection and downstream detrimental cytopathic effects that the SARS-CoV-2 may be able to replicate in distinct perinuclear locations within hiPSC-CMs co-opting cellular organelles for viral protein translation, and that SARS-CoV-2 infection reduces functional contractility in hiPSC-CMs.

### Example 13

### Employ hiPSC-CMs and Microfluidic Organ Chips to Establish a Cardiac-Specific Antiviral Drug Screening Platform Against SARS-CoV-2

A number of repurposed and de-novo drugs are currently being investigated for their ability to reduce SARS-CoV-2 viral replication and thus alleviate COVID-19 symptoms (Table 8). Given the possibility of cardiac-specific infection by SARS-CoV-2, The Inventors aim to establish an hiPSC-CM-based platform by which candidate antiviral compounds can be screened for their efficacy. This is piggybacking upon The Inventors’ previous study, which demonstrated that interferon beta, ribavirin, and other known antivirals can significantly reduce coxsackievirus proliferation in vitro in a dose-dependent fashion. Similarly, The Inventors will infect hiPSC-CMs with SARS-CoV-2 and test the ability of candidate drugs to stymie infection using a high-throughput timecourse and dose response approach. The Inventors will subsequently conduct transcriptomic analysis of infected cells treated with candidate antivirals, to determine the mechanism of antiviral action for each of these compounds.

TABLE 8

Drugs repurposed for potential COVID-19 treatment

| | |
|---|---|
| Drug | Mechanism of Action |
| interferon beta | Activation of viral RNA and protein clearance pathways |
| ribavirin | Nucleoside inhibitor of viral RNA synthesis |
| chloroquine | endosomal and lysosomal regulation; zinc ionophore |
| azithromycin | Antibiotic |
| favipiravir | inhibition of viral RNA-dependent RNA polymerase |
| lopinavir/ritonavir | Protease inhibitor |
| remdesivir | nucleotide analog, interferes with viral RNA polymerase |

Together, these studies will provide confirmation of cardiac-specific antiviral efficacy for candidate drugs being used to treat patients with COVID-19. Such a platform could also be used to test de-novo antivirals under active development.

### Example 14

### Platforms to Test Viral Infection Using Organoids

3D organoid culture: organoids may be directly tested with viral infection or drug screening

Regional lung organoid models - distal human lung organoids: unfractionated distal lung epithelial cells organoids

Distal organoids are infected either in situ or after Dispase (protease which cleaves fibronectin, collagen IV, and to a lesser extent collagen I) treatment to remove the Matrigel®.

Regional lung organoid models – proximal human lung organoids: using epithelial cells isolated from trachea-bronchial regions of human lung tissue.

Air-Liquid Interface (ALI) cultures: alternative culturing platform

Differentiated air-liquid interface (ALI) cultures - tracheo-bronchial ALI cultures. Tracheo-bronchial epithelial cells are isolated as above and expanded in 2D culture on collagen-coated plates. After expansion to P1-5 these cells can either be cryobanked or seeded (4 × 10⁴ - 1 × 10⁵) into collagen-coated TransWells to prepare ALI cultures.

Differentiated air-liquid interface (ALI) cultures - small airway ALl cultures.

Methods have been developed for the enrichment of small airway epithelium from dissociated distal lung tissue. Work is in progress to develop and validate small airway ALI cultures.

### Technical Information

### 3D Organoid Culture

Regional lung organoid models - distal human lung organoids: For the current study the Inventors are using unfractionated distal lung epithelial cells for simplicity. Distal lung epithelial cells are recovered from dissociated tissue by FACS, selecting viable cells based upon exclusion of the DNA dye propidium iodide and subsequently using depletion of CD45 and CD31 positive cells and positive selection of CD326 positive cells for enrichment of the epithelial fraction. The Inventors have the potential to further fractionate this population of epithelial cells into alveolar and small airway, based upon either positive or negative selection of cells with the HTII-280 monoclonal antibody. With sufficient tissue and isolated cells this would have been the Inventors’ preferred approach as it would allow modeling of both alveolar and small airway compartments. For each organoid culture well, distal lung epithelial cells are then mixed with culture expanded MRC5 lung fibroblasts (5 × 10³ epithelial cells, 7.5 × 10⁴ MRC5 cells) in a total volume of 100 ul and 50 ul of growth factor depleted MatriGelⓇ added prior to mixing and placing in a 6.5 mm TransWell. Organoids are cultured for 7-10 days in expansion medium (Pneumacult ALI medium supplemented with a Wnt agonist [CHIR] and Tgf-beta inhibitor [SB431542]) prior to differentiating in the same medium lacking either Wnt agonist or Tgf-beta inhibitor for 4-7 days – medium is placed in the lower compartment of TransWell cultures with the upper surface of polymerized MatriGel® at the air interface. These cultures will be infected with SARS-CoV2 by topical application to the culture inserts harboring organoids.

Regional lung organoid models – proximal human lung organoids: These are prepared as above but using epithelial cells isolated from trachea-bronchial regions of human lung tissue. Infection with SARS-CoV2 will be performed as above.

Air-Liquid Interface (ALI) cultures: culture methods themselves have been established in the literature.

Differentiated air-liquid interface (ALI) cultures - tracheo-bronchial ALI cultures. Tracheo-bronchial epithelial cells are isolated as above and expanded in 2D culture on collagen-coated plates. After expansion to P1-5 these cells can either be cryobanked or seeded (4 × 10⁴ - 1 × 10⁵) into collagen-coated TransWells to prepare ALI cultures. The seeded epithelial cells will be allowed to expand to confluence in submerged cultures until confluence using the Pneumacult Ex or Ex-plus media supplemented with hydrocortisone and Rho kinase inhibitor, Y27632, after which time media are removed from the apical compartment of cultures. The media in the basal compartment is changed to Pneumacult ALI media supplemented with hydrocortisone and heparin and the epithelial cells are allowed to differentiate at the air interface to yield a mature pseudostratified epithelium (typically after 14-28 days of culture at air interface). Experimental infection with SARS—CoV2 will be accomplished by application of virus to either apical or basolateral surfaces of ALI cultures.

Differentiated air-liquid interface (ALI) cultures – small airway ALI cultures. In the process. These culture models are being developed and validated at present. They are being developed from distal lung HTII-280-negative cells described above. ALI cultures and infection with SARS-CoV2 will be as detailed above.

### Example 15A

### Cell Isolation

Human lung tissue was obtained from deceased organ donors in compliance with consent procedures developed by International Institute for the Advancement of Medicine (IIAM) and approved by the Internal Review Board at Cedar-Sinai Medical Center.

Human lung tissue was processed as described previously with the following modifications. For isolation of proximal airway cells, trachea and the first 2-3 generation of bronchi were slit vertically and enzymatically digested with Liberase (50 µg/mL) and DNase 1 (25 µg/mL) incubated at 37° C. with mechanical agitation for 20 minutes, followed by gentle scraping of epithelial cells from the basement membrane. The remaining tissue was finely minced and further digested for 40 minutes at 37° C. For distal alveolar cell isolation, small airways of 2 mm diameter or less and surrounding parenchymal tissue was minced finely and enzymatically digested for 40-60 minutes as described before. Total proximal or distal dissociated cells were passed through a series of cell strainers of decreasing pore sizes from 500 µm to 40 pm under vacuum pressure and depleted of immune and endothelial cells by magnetic associated cell sorting (MACS) in accordance to the manufacturing protocol (Miltenyi Biotec). Viable epithelial cells were further enriched by fluorescence associated cell sorting (FACS) using DAPI (Thermo Fisher Scientific) and antibodies against EPCAM (CD326), CD45 and CD31 (Biolegend) on a BD Influx cell sorter (Becton Dickinson).

### Culture and Differentiation of Proximal Airway Epithelial Cells at Air Liquid Interface

FACS enriched proximal airway epithelial cells were expanded in T25 or T75 flasks coated with bovine type I collagen (Purecol, Advanced biomatrix) in Pneumacult Ex media (STEMCELL Technologies), supplemented with 1X Penicillin-Streptomycin-Neomycin (PSN) Antibiotic Mixture (Thermo Fisher Scientific) and 10 µM Rho kinase inhibitor, Y-27632 (STEMCELL technologies). Upon confluence cells were dissociated using 0.05% Trypsin-EDTA (Thermo Fisher Scientific) and seeded onto collagen coated 0.4 µm pore size transparent cell culture inserts in a 24-well supported format (Coming) at a density of 7.5 × 10⁴ cells per insert. Cells were initially cultured submerged with 300 µL of Pneumacult Ex media in the apical chamber and 700 µL in the basement chamber for 3-5 days. Upon confluence, cells were cultured at air liquid interface in 700 µL Pneumacult ALT media (STEMCELL Technologies) supplemented with 1X PSN and media was changed every 48 hrs. Cultures were maintained at 37° C. in a humidified incubator (5% CO2) and used for SARS-CoV-2 infection after 16-20 days of differentiation.

### Culture of 3D Alveolar Organoids

Five thousand FACS enriched distal lung epithelial cells were mixed with 7.5 × 10⁴ MRC5 human lung fibroblast cells (ATCC CCL-171) and resuspended in a 50:50 (v/v) ratio of ice cold Matrigel® (Coming) and Pneumacult ALT medium. 100 uL of the suspension was seeded onto the apical surface of a 0.4 µm pore-size cell culture insert in a 24 well supported format. After polymerization of Matrigel® (RP, 700 µL of Pneumacult ALT medium was added to the basement membrane. Media was supplemented with 50 µg per ml of Gentamycin (Sigma Aldrich) for the first 24 hrs. and 10 µM Rho kinase inhibitor for the first 48 hrs. 2 µM of the Wnt pathway activator, CHTR-99021 (STEMCELL technologies) was added to the media at 48 hrs and maintained for the entire duration of culture. Media was changed every 48 hrs. Cultures were maintained at 37° C. in a humidified incubator (5% CO2) and used for SARS-Co-V2 infection after 15 days.

### SARS-CoV-2 Infection of ALI and Organoid Cultures

All studies involving SARS-CoV-2 infection of proximal and distal lung epithelial cells were conducted in the UCLA BSL3 High-Containment Facility. SARS-CoV-2, isolate USA-WA1/2020, was sourced from the Biodefense and Emerging Infections (BEI) Resources of the National Institute of Allergy and Infectious Diseases (NIAID). SARS-CoV-2 was amplified once in Vero-E6 cells and viral stocks were stored at -80° C. Vero-E6 cells were cultured in Eagle’s minimal essential medium (MEM) (Coming) supplemented with 10% fetal bovine serum (Coming), penicillin-streptomycin (100 units/ml, Gibco), 2 mM L-glutamine (Gibco) and 10 mM HEPES (Gibco). Cells were incubated at 37° C. in a humidified incubator (5% CO₂).

Prior to infection of alveolar organoid cultures, Matrigel® was dissolved by adding 500µL of Dispase (500 µg/ml, Gibco) to the apical and basement chambers of inserts and incubating for 1 hour at 37° C. Organoids were harvested, washed, gently triturated with a P1000 tip by pipetting up and down. SARS-CoV-2 inoculum (1×10^4 TCID50 per well) was added to the alveolar organoids in a 2 ml conical tube and incubated for 2 hours at 37° C. (5% CO2). Every 15 minutes, tubes were gently mixed to facilitate virus adsorption on to the cells. Subsequently, inoculum was replaced with fresh Pneumacult ALI medium and organoids were transferred to the apical chamber of the insert in 100 µL volume with 500 µL of the medium in the basement chamber. Cultures were incubated at 37° C. (5% CO2) and harvested at indicated time points for sample collection. For infection of proximal airway ALI cultures, 100 µl of SARS-CoV-2 virus inoculum (1×10^4 TCID50 per well) was added on to the apical chamber of inserts. Cells were incubated at 37° C. (5% CO2) for 2 hours for virus adsorption. Subsequently, the cells were washed and fresh Pneumacult media (500 µl in the base chamber) was added. Cells were incubated at 37° C. (5% CO2 and harvested for analysis at indicated time points for sample collection.

### Drug Validation Experiments

Hydroxychloroquine (Selleck Chemicals Cat. No. S4430) and Remdesivir (Selleck Chemicals Cat. No. S8932) were dissolved in DMSO to a stock concentration of 10 mM. IFNB1 stock of 10′6 units/ml was provided by Dr. Jay Kolls. To test the efficacy of the drugs against SARS-CoV-2 infection/replication in proximal airway epithelial cells and AT2 cells, cultures were treated with 10 µM of Hydroxychloroquine or Remdesivir, or 100 units/ml of IFNB1.

For treatment of proximal airway epithelial cells, Pneumacult ALI media containing drugs was added to ALI cultures (100 µL to the apical chamber and 700 µL to the basement chamber), 3 hours prior to infection. 100 µL of SARS-CoV-2 viral inoculum (1×10⁴ TCID50 per well) was added to the apical chamber. After 2 hours of viral adsorption, cells were washed and 700 µL of fresh Pneumacult ALI media containing drugs was added to the basement chamber. Drugs were maintained in the media for the duration of culture post infection. ALI cultures without drug treatment in the presence or absence (mock) of viral infections were included as controls.

For alveolar organoid drug study, dissociated organoids were suspended in 100 µL of Pneumacult ALI media containing drugs 3 hours prior to SARS-CoV-2 infection. Viral infections were performed as described previously. Drugs were maintained in the media for the duration of culture post infection. Organoid cultures without drug treatment in the presence or absence (mock) of viral infections were included as controls.

### Immunofluorescence Staining

1-3 days after SARS-CoV-2 infection, cultures were fixed by adding 500 µL of 4% paraformaldehyde in phosphate-buffered saline (PBS) for 20 minutes. Fixed samples were permeabilized and blocked for 1 hour in a ‘blocking buffer’ containing PBS, 2% bovine serum albumin, 5% goat serum, 5% donkey serum and 0.3% Triton X-100. Primary antibodies diluted in the blocking buffer were applied to samples and incubated overnight at 4° C. The following primary antibodies were used: Acetylated tubulin (1:200, Sigma Aldrich, Cat. No. T6793); Mucin 5AC, 45M1 (1:500, Thermo Fisher Scientific, Cat. No. MA5-12178); HTII-280 (1:500, Terrace Biotech, Cat. No. TB-27AHT2-280); SARS-CoV-2 spike (S) protein (1:100, BEI Resources NR-616 Monoclonal Anti-SARS-CoV S Protein (Similar to 240C) SARS coronavirus); Cleaved caspase-3 (1:200, Cell Signaling Technology Cat. No. 9661). Samples were washed 3 times for 5 minutes each with PBS. Appropriate secondary antibodies conjugated to fluorophores (Thermo Fisher Scientific) were applied to the samples for 1 hour at room temperature. Samples were washed 3 times for 5 minutes each with PBS followed by addition of DAPI (1:5000) for 5 minutes. Insert membranes were carefully detached from their transwell support with a fine scalpel, and transferred to a glass microscopic slide. Samples were mounted using Fluoromount G (Thermo Fisher Scientific), imaged on a Zeiss LSM 780 Confocal Microscope and images were processed using Zen Blue software (Zeiss).

### Real Time Quantitative PCR (RT-qPCR)

Total RNA was extracted from mock and SARS-CoV-2 infected proximal airway ALI and alveolar organoid cultures lysed in Trizol (Thermo Fisher Scientific) using the chloroform-iso-propanol-ethanol method. 500 ng of RNA was reversed transcribed into cDNA in a 20 µL reaction volume using iScript cDNA synthesis kit (Biorad) in accordance to manufacturer’s guidelines. RT-qPCR was performed on 10 ng of CDNA per reaction in triplicates for each sample using SYBR green master mix (Thermo Fisher Scientific) on a 7500 Fast Real Time PCR system (Applied Biosystems). Primers sequences for detection of SARS-CoV-2 N gene were obtained from the Center for Disease Control’s resources for research labs. Primer pairs used are as follows:

2019-nCoV _N1 Forward: GAC CCC AAA ATC AGC GAA AT (SEQ ID NO:1)
2019-nCoV _N1 Reverse: TCT GGT TAC TGC CAG TTG AAT CTG (SEQ ID NO:2)
GAPDH Forward: CCACCTTTGACGCTGGG (SEQ ID NO:3)
GAPDH Reverse: CATACCAGGAAATGAGCTTGACA (SEQ ID NO: 4)

### Bulk RNA sequencing

2 days after SARS-CoV-2 infection, total RNA was extracted for bulk RNA sequencing as described before. RNA quality was analyzed using the 2100 Bioanalyzer (Agilent Technologies and quantified using QubitTM (ThermoFisher Scientific). Library construction was performed by the Cedars-Sinai Applied Genomics, Computation and Translational Core using the Lexogen RiboCop rRNA Depletion kit and Swift Biosciences RNA Library Sciences. Sequencing was performed using the NovaSeq 6000 (Illumina) with single-end 75 bp sequencing chemistry. On average, about 20 million reads were generated from each sample. Raw reads were aligned using Star aligner 2.6.1/RSEM 1.2.28 with default parameters, using a custom human GRCh38 transcriptome reference downloaded from www.gencodegenes.org, containing all protein coding and long non-coding RNA genes based on human GENCODE version 33 annotation with SARS-Cov2 virus genome MT246667.1 www.ncbi.nlm.nih.gov/nuccore/MT246667.1.

Differential gene expression was determined by DESeq2. Top differential genes were determined based on fold change and test statistics. Pathway analysis was performed using Ingenuity Pathway Analysis.

### Statistics

Statistical analysis was performed using GraphPad Prism version 8 software. Data is presented as linear fold change or log2 fold change ± SEM. SARS-CoV-2 infection time course for ALI and organoid cultures was analyzed using Two-Way ANOVA with Sidak’s post hoc correction. Drug validation experiments were analyzed using One-way ANOVA with Tukey’s post hoc correction.

### SARS-CoV-2 Infects and Replicates Within Human Proximal Airway Epithelial Cells

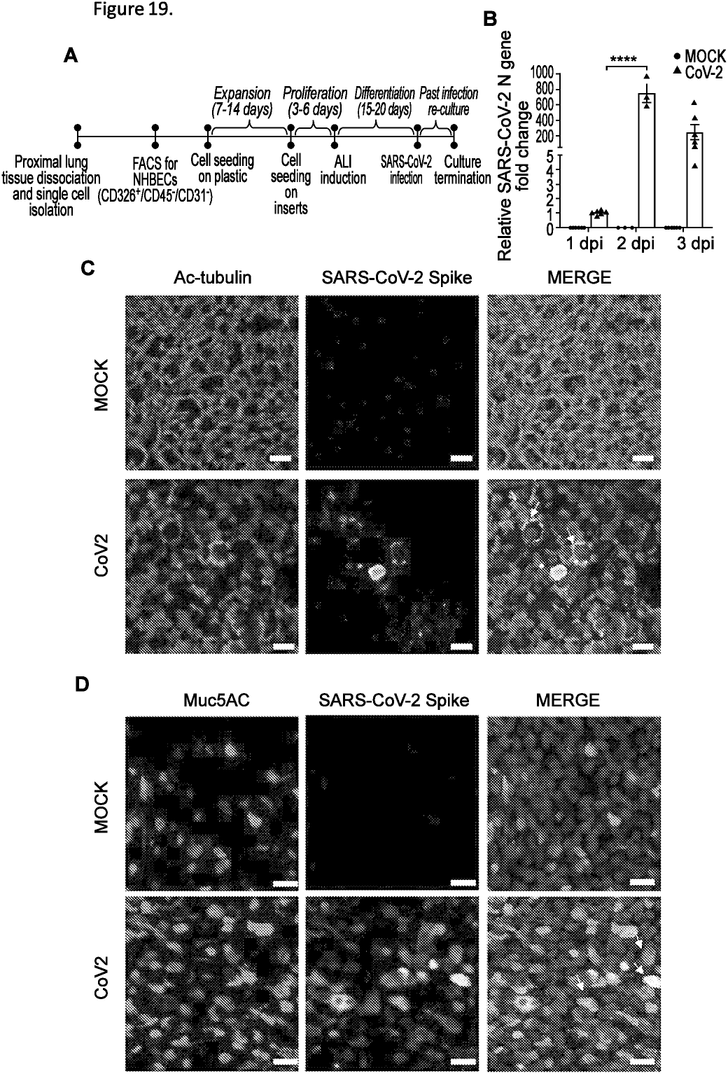

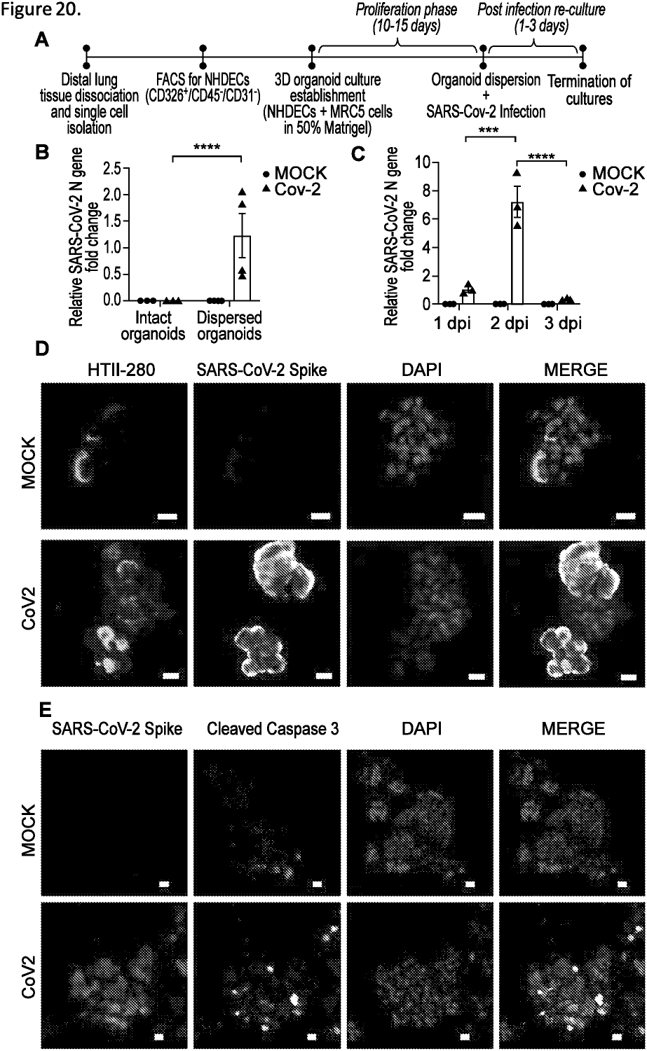

Since the upper respiratory tract represents the most likely initial site of respiratory virus infection, we initially utilized the well-established air-liquid interface (ALI) culture system to study the effect of SARS-CoV-2 infection on proximal airway epithelial cells (FIG. 19A). Human trachea-bronchial epithelial cells (HBECs) isolated from trachea and upper bronchi were differentiated at ALI for 16-20 days to yield a pseudostratified mucociliary epithelium and infected with SARS- CoV-2 (1×10^4 TCID50 per well). Cells were harvested for analysis at 1 to 3 days post infection (dpi) (FIG. 19A). SARS-CoV-2 readily infected well-differentiated proximal airway cells leading to viral replication and gene expression as indicated by induction of SARS-CoV-2 Nucleoprotein gene (N gene) RNA in infected samples. N gene abundance, reflective of either viral mRNA expression or a result of genome replication increased 750-fold at 2 dpi and declined at 3 dpi, however, the later decline was not statistically significant. Viral N gene expression was undetectable in corresponding mock cultures at all 3 time points (FIG. 19B).

Further analysis of infected cultures revealed that SARS-CoV-2 infection of proximal airway ALI cultures was heterogenous. SARS-CoV-2 predominantly targeted ciliated cells, evidenced by colocalization of SARS-CoV-2 viral capsid “spike” protein with the ciliated cell marker, acetylated tubulin (FIG. 19C). This observation is congruent with previous reports of infection of ciliated cells by SARS-CoV and recent reports of infection of ciliated cells by SARS-CoV-2, in in vitro cultures of proximal airways as well as COVID-19 patient biopsies. A number of studies have described the expression of the SARS-CoV-2 entry receptor, angiotensin-converting enzyme2 (ACE2) in the respiratory tracts. In the nasal passages and large airways highest levels of ACE2 expression have been reported in ciliated cells. However, we also observed SARS-CoV-2 infection within a small proportion of goblet cells, expressing the mucin, Muc5ac (FIG. 19D).

### SARS-CoV-2 Infects and Replicates Within Human Alveolar Type 2 Cells

A number of recent studies have utilized various cell lines, primary epithelial cultures of nasal and proximal airway epithelium to study SARS-CoV-2 infection of the upper airways, the initial site of entry and infection. However, there is a lack of a tractable system to model SARS-CoV-2 infection in the distal gas-exchange region of the lung, the alveolus, which represents the site of severe pathology and pneumonia. Recent single cell transcriptome studies have shown that ACE2, the host cell surface receptor for SARS-CoV-2 attachment and infection, is predominantly expressed by alveolar type 2 (AT2) cells. AT2 cells are bifunctional cells that serve both as progenitors that contribute to epithelial maintenance in addition to fulfilling specialized functions such as surfactant production. We established a 3D organoid culture model of the human alveoli to model SARS-CoV-2 infection of the distal lung in in vitro (FIG. 20A). This is the first study to report the development of a primary in vitro model of the adult human alveoli to study the pathogenesis of SARS-CoV-2 infection. Using this system, primary epithelial cells isolated from normal human distal lung tissue were cultured in the presence of stromal support cells leading to the generation of alveolar organoids. Organoid cultures were exposed to SARS-CoV-2 (1×10^4 TCID50 per well) and harvested for analysis 1-3 dpi. We found that intact organoids were refractory to viral infection but gentle physical and enzymatic disruption was permissive for viral infection and replication, as assessed by relative N gene expression (FIG. 20B). Disruption of organoids exposed the apical cell surfaces of cells that otherwise face inwards towards the air-filled lumen in the 3D organoid structure, thus presumably enabling the virus to access its entry receptor, ACE2. Study of viral infection kinetics from 1 dpi to 3 dpi demonstrated that viral infection peaked at 2 dpi. N gene expression at 2 dpi was 7-fold higher compared to the mean infection at 1 dpi and declined significantly by 3 dpi. Viral N gene expression was below the detection limits in corresponding mock cultures at all 3 time points (FIG. 20C).

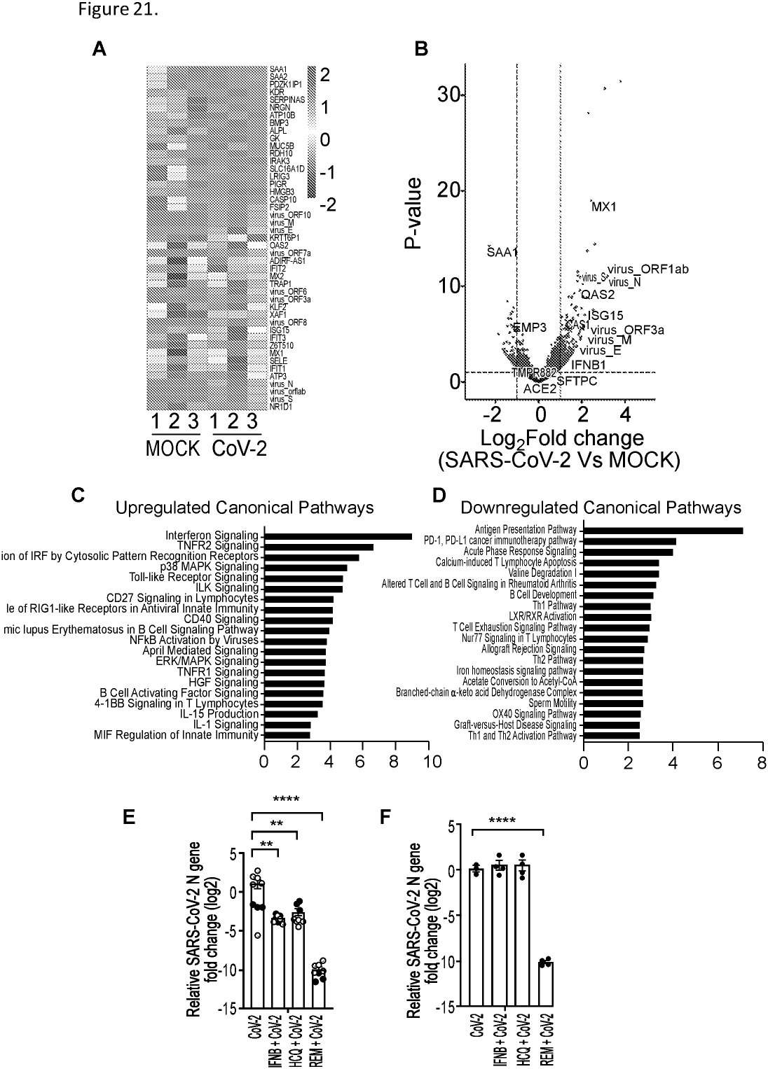

We used immunofluorescence confocal microscopy to confirm infection of organoid cultures with SARS-CoV-2. Mock and infected alveolar organoids were stained for the well-known AT2 cell marker, HTII-280 and SARS-CoV-2 spike protein. Organoid cultures showed abundant expression of HTII-280 and in infected cultures, viral spike protein localized to the HTII-280⁺ AT2 cells (FIG. 20D). By 3dpi, infected alveolar organoids exhibited significantly enhanced staining for the apoptotic marker, cleaved caspase-3 compared to mock cultures. Interestingly only a proportion of the apoptotic cells were infected, thus demonstrating that SARS-CoV-2 induced a cytopathic effect on infected AT2 cells as well as neighboring uninfected cells (FIG. 20E, FIG. 21). SARS-CoV-2 induced apoptosis of neighboring uninfected epithelial cells is suggestive of the potential for a non-cell autonomous effect on alveolar epithelial integrity.

Taken together, these data demonstrate that AT2 cells are susceptible to SARS-CoV-2 infection and suggest that infection triggers both cell-autonomous and non-cell-autonomous apoptosis that may contribute to alveolar injury. We conclude that 3D alveolar organoid cultures serve as a robust platform for studying the effect of SARS-CoV-2 infection on adult distal lung alveolar epithelium.

### 3D Alveolar Organoid Cultures as a Tool to Study Host Response to SARS-CoV-2 Infection

Having established a 3D alveolar organoid model that enables robust infection of AT2 cells by SARS-CoV-2, we further evaluated the utility of this system to study host pathogen responses. Distal organoid cultures were infected with SARS-CoV-2 and cells were harvested 2 dpi for transcriptomic analysis via global RNA-Sequencing. Transcript profiles were compared between SARS-CoV-2 infected and mock organoid cultures. Heatmap (FIG. 21A) and volcano plots (FIG. 21B) of differentially expressed genes revealed detection of high levels of SARS-CoV-2 viral RNA such as coding sequences for Virus_N, virus ₋ORFlab, virus ₋ORF3ab, further confirming SARS-CoV-2 genome replication and/or gene transcription in organoid cultures. Furthermore, infected organoids showed robust induction of host response genes including cytokines such as IFNB1 and antiviral response genes OAS1, OAS2, ISG15 and MX1. We did not see a significant change in expression of the AT2 cell marker, SFTPC, or host genes involved in viral infection such as ACE2 and TMPRSS2. Analysis of differentially regulated canonical pathways revealed that Interferon (IFN) signaling was the most upregulated pathway. Other upregulated pathways included NF-kB activation, TLR signaling and IL1 signaling (FIG. 21C). On the other hand, antigen presentation, Th1 and Th2 activation pathways were downregulated (FIG. 21D). Taken together, these data indicate that SARS-CoV-2 infection induces significant alteration of innate immune response genes in alveolar cells without the participation of recruited immune cells. We speculate that the epithelial innate immune response may provide activating signals leading to global activation of the host immune response and provide therapeutic targets for mitigation of uncontrolled lung inflammation and adverse patient outcomes.

### Organoid and ALI Culture Systems as a Tool to Screen Therapeutic Targets Against SARS-CoV-2 Infection

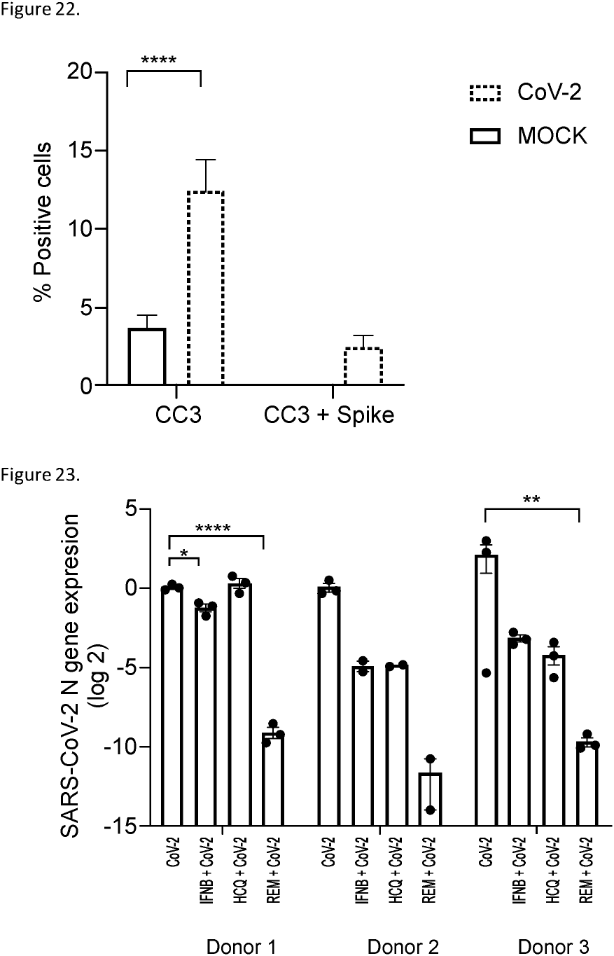

Given that animal models such as mice are not natural hosts to SARS-CoV-2 infection, there is a vital need for development of alternate pre-clinical models that closely recapitulate the human lung for screening potential therapeutic agents that target SARS-CoV-2 infection and replication. We utilized the 3D alveolar organoid and proximal airway ALI culture systems to study the effect of a selected panel of drugs which included the known anti-viral cytokine, IFNB1 and investigational drugs for COVID-19 treatment, Remdesivir and Hydroxychloroquine. Treatment of 3D alveolar organoid cultures with IFNB1 lead to a 3.2-log reduction in viral N gene RNA compared to control infected organoids. Hydroxychloroquine led to an overall 2.4-log reduction in viral N gene expression compared to average infection in untreated organoids (FIG. 3E). However, variable effects of hydroxychloroquine were observed on viral replication/gene expression that were donor epithelium-dependent (FIG. 22). Remdesivir showed the strongest effect on viral replication in alveolar organoids, resulting in a 9-log decrease in viral N gene expression compared to the average infection in untreated organoids (FIG. 21E). This effect was consistent irrespective of donor origin of epithelial cells confirming it as a direct acting antiviral (DAA) agent targeting viral specific RNA polymerase (FIG. 22). IFNB1 and Hydroxychloroquine did not show a significant effect on viral N gene RNA in proximal ALI cultures. Proximal airway ALI cultures used for this study were derived from separate donors compared to alveolar organoid cultures. The lack of response to IFNB1 may reflect differences in disease susceptibility observed in different COVID-19 patients. However, further analysis is warranted to confirm this observation. However, Remdesivir resulted in a 10.2-log reduction in viral N gene RNA abundance, confirming its effect as a DAA (FIG. 3F). Taken together, our data show that 3D alveolar organoid models and proximal ALI cultures represent a highly relevant preclinical tool to assess SARS-CoV-2 infection and replication, and serve as a sensitive platform for drug screening and validation. Overall, our study provides a novel model to investigate SARS-CoV-2 infection along the proximal-distal axis of the human lung epithelium. SARS-CoV-2 infection is accompanied by a cell-autonomous proinflammatory response and viral infection and/or replication is strongly suppressed by the candidate drug, Remdesivir. This platform can help better understand the pathogenesis of COVID-19. Furthermore, our platform provides a relevant preclinical model for rapid screening of drugs against COVID-19 and future emergent respiratory pathogens.

### Example 15B

### Tissue Procurement

Human lung tissue was obtained from ‘normal’ (without prior history of lung disease) deceased organ donors in compliance with consent procedures developed by International Institute for the Advancement of Medicine (IIAM) and approved by the Internal Review Board at Cedar-Sinai Medical Center.

Donor 1: 52-year-old Male; Donor 2: 37-year-old Male; Donor 3: 60-year-old Female; Donor 4: 78-year-old Female; Donor 5:18 year old Male; Donor 6: 78-year-old Female; Donor 7: 72-year-old Female 10.

### Primary Cell Cultures

Human proximal airway cells (tracheo-bronchial epithelial cells) and distal lung small airway epithelial cells were isolated from freshly excised normal human lungs obtained from transplant donors with lungs unsuitable for transplant, procured from IIAM under IRB-approved protocol.

Proximal cells expanded in Pneumault Ex medium and differentiated in Pneumacult ALI medium as described in methods details. Distal small airway epithelial cells were cultured as 3D alveopsheres in Pneumacult ALI medium as described in method details.

### Virus Strain

SARS-CoV-2, isolate USA-WA1/2020 (NR-52281), was sourced from the Biodefense and Emerging Infections (BEI) Resources of the National Institute of Allergy and Infectious Diseases (NIAID, GenBank: MT020880) All studies involving SARS-CoV-2 infection of proximal airway and AT2 cells were conducted in the UCLA BSL3 High-Containment Facility. SARS-CoV-2 was amplified once in Vero-E6 cells and viral stocks were stored at -80 oC. Vero-E6 cells were cultured in Eagle’s minimal essential medium (MEM) (Coming) supplemented with 10% fetal bovine serum (Corning), penicillin-streptomycin, L-glutamine (Gibco) and HEPES (Gibco). Specifics of viral culture medium are described in method details.

### Cell Isolation

Human lung tissue was processed as described previously with the following modifications. For isolation of proximal airway cells, trachea and the first 2-3 generation of bronchi were slit vertically and enzymatically digested with Liberase (50 µg/mL) and DNase 1 (25 µg/mL) incubated at 37° C. with mechanical agitation for 20 minutes, followed by gentle scraping of epithelial cells from the basement membrane. The remaining tissue was finely minced and further digested for 40 minutes at 37° C. For distal alveolar cell isolation, small airways of 2 mm diameter or less and surrounding parenchymal tissue was minced finely and enzymatically digested for 40-60 minutes as described before. Total proximal or distal dissociated cells were passed through a series of cell strainers of decreasing pore sizes from 500 µm to 40 µm under vacuum pressure and depleted of immune and endothelial cells by magnetic associated cell sorting (MACS) in accordance to the manufacturing protocol (Miltenyi Biotec). Viable epithelial cells were further enriched by fluorescence associated cell sorting (FACS) using DAPI (ThermoFisher Scientific) and antibodies against EPCAM (CD326), CD45 and CD31 (Biolegend) on a BD Influx cell sorter (Becton Dickinson).

### Culture and Differentiation of Proximal Airway Epithelial Cells at Air Liquid Interface

FACS enriched proximal airway epithelial cells were expanded in T25 or T75 flasks coated with bovine type I collagen (Purecol, Advanced biomatrix) in Pneumacult Ex media (STEMCELL Technologies), supplemented with 1X Penicillin-Streptomycin-Neomycin (PSN) Antibiotic Mixture (Thermo Fisher Scientific) and 10 µM Rho kinase inhibitor, Y-27632 (STEMCELL technologies). Upon confluence cells were dissociated using 0.05% Trypsin-EDTA (ThermoFisher Scientific) and seeded onto collagen coated 0.4 µm pore size transparent cell culture insert in a 24-well supported format (Coming) at a density of 7.5 × 10⁴ cells per insert. Cells were initially cultured submerged with 300 µL of Pneumacult Ex media in the apical chamber and 700 µL in the basement chamber for 3-5 days. Upon confluence, cells were cultured at air liquid interface in 700 µL Pneumacult ALI media (STEMCELL Technologies) supplemented with 1X PSN and media was changed every 48 hrs. Cultures were maintained at 37° C. in a humidified incubator (5% CO₂) and used for SARS-CoV-2 infection after 16-20 days of differentiation.

### Culture of 3D Alveospheres

Five thousand FACS enriched distal lung epithelial cells were mixed with 7.5 × 104 MRC5 human lung fibroblast cells (ATCC CCL-171) and resuspended in a 50:50 (v/v) ratio of ice cold Matrigel® (Coming) and Pneumacult ALI medium. 100 uL of the suspension was seeded onto the apical surface of a 0.4 µm pore-size cell culture insert in a 24 well supported format. After polymerization of Matrigel®, 700 µL of Pneumacult ALI medium was added to the basement membrane. Media was supplemented with 50 µg per ml of Gentamycin (Sigma Aldrich) for the first 24 hrs. and 10 µM Rho kinase inhibitor for the first 48 hrs. 2 µM of the Wnt pathway activator, CHIR-99021 (STEMCELL technologies) was added to the media at 48 hrs and maintained for the entire duration of culture. Media was changed every 48 hrs. Cultures were maintained at 37° C. in a humidified incubator (5% CO₂) and used for SARS-Co-V2 infection after 15-20 days.

### SARS-CoV-2 Infection of Proximal ALl and Alveolar Type 2 Cultures

All studies involving SARS-CoV-2 infection of proximal airway and AT2 cells were conducted in the UCLA BSL3 High-Containment Facility. SARS-CoV-2, isolate USA-WA1/2020, was sourced from the Biodefense and Emerging Infections (BEI) Resources of the National Institute of Allergy and Infectious Diseases (NIAID). SARS-CoV-2 was amplified once in Vero-E6 cells and viral stocks were stored at -80 oC. Vero-E6 cells were cultured in Eagle’s minimal essential medium (MEM) (Coming) supplemented with 10% fetal bovine serum (Coming), penicillin-streptomycin (100 units/ml, Gibco), 2 mM L-glutamine (Gibco) and 10 mM HEPES (Gibco). Cells were incubated at 37° C. in a humidified incubator (5% CO₂). Prior to infection of AT2 cultures, Matrigel® was dissolved by adding 500 µL of Dispase (500 µg/ml, Gibco) to the apical and basement chambers of inserts and incubating for 1 hour at 37° C.

Alveospheres were harvested, washed, gently dispersed with a P1000 tip by pipetting up and down 3 times such that the alveospheres were ‘popped open’, exposing the apical surface of the cells. Hereafter the cultures were referred to as AT2 cultures. SARS-CoV-2 inoculum (1×10^4 TCID50 22 per well) was added to the AT2 cultures in a 2 ml conical tube and incubated for 2 hours at 37° C. (5% CO₂). Every 15 minutes, tubes were gently mixed to facilitate virus adsorption on to the cells. Subsequently, inoculum was replaced with fresh Pneumacult ALI medium and AT2 cultures were transferred to the apical chamber of the insert in 100 µL volume with 500 µL of the medium in the basement chamber. Cultures were incubated at 37° C. (5% CO₂) and harvested at indicated time points for sample collection.

For infection of proximal airway ALI cultures, 100 µl of SARS-CoV-2 virus inoculum (1×10^4 TCID50 per well) was added on to the apical chamber of inserts. Cells were incubated at 37° C. (5% CO₂) for 2 hours for virus adsorption. Subsequently, the cells were washed and fresh Pneumacult ALI media (500 µl in the base chamber) was added. Cells were incubated at 37° C. (5% CO₂ and harvested for analysis at indicated time points for sample collection.

### Viral Titres

AT2 cultures were infected with 1×10^4 TCID50 per well of SARS-CoV-2 as described before. At each timepoint (1,2 and 3 dpi), media from the basal chamber from mock and SARS-CoV-2 infected wells were collected and stored at -80° C. Viral production by infected AT2 cultures was measured by quantifying TCID50 (Median Tissue Culture Infectious Dose). Briefly, Vero-E6 cells were plated in 96-well plates at a density of 5 ×10³ cells/well. The following day, samples collected from AT2 cultures at the different timepoints were subjected to 10-fold serial dilutions (10⁻¹ to 10⁻⁶) and inoculated onto Vero-E6 cells. The cells were incubated at 37° C. with 5% CO2. After 72 hours, each inoculated well was examined for presence or absence of viral CPE and percent infected dilutions immediately above and immediately below 50% were determined. TCID50 was calculated based on the method of Reed and Muench.

### Drug Validation Experiments

Hydroxychloroquine (Selleck Chemicals Cat. No. S4430) and Remdesivir (Selleck Chemicals Cat. No. S8932) were dissolved in DMSO to a stock concentration of 10 mM. IFNB1 stock of 10^6 units/ml was provided by Dr. Jay Kolls. To test the efficacy of the drugs against SARS-CoV-2 infection/replication in AT2 cells, cultures were suspended in media containing treated with 10 µM of Hydroxychloroquine or Remdesivir, or 100 units/ml of IFNB1 3 hours prior to SARS-CoV-2 infection. Viral infections were performed as described previously. Drugs were maintained in the media for the duration of culture post infection. AT2 cultures without drug treatment in the presence or absence (mock) of viral infections were included as controls.

### Immunofluorescence Staining

1-3 days after SARS-CoV-2 infection, cultures were fixed by adding 500 µL of 4% paraformaldehyde in phosphate-buffered saline (PBS) for 20 minutes. Fixed samples were permeabilized and blocked for 1 hour in a ‘blocking buffer’ containing PBS, 2% bovine serum albumin, 5% goat serum, 5% donkey serum and 0.3% Triton X-100. Primary antibodies diluted in the blocking buffer were applied to samples and incubated overnight at 4° C. The following primary antibodies were used: FOXJ1 (1:300, Thermo Fisher Scientific, Cat. No. 14-9965-82); Mucin 5AC, 45M1 (1:500, Thermo Fisher Scientific, Cat. No. MA5-12178); HTII-280 (1:500, Terrace Biotech, Cat. No. TB-27AHT2-280); HTI-56 (1:100, Terrace Biotech, Cat. No. TB-29AHT1-56) SARS-CoV-2 spike (S) protein (1:100, BEI Resources Polyclonal Anti-SARS Coronavirus (antiserum, Guinea Pig), NR-10361); Cleaved caspase-3 (1:200, Cell Signaling Technology Cat. No. 9661). Samples were washed 3 times for 5 minutes each with PBS. Appropriate secondary antibodies conjugated to fluorophores (Thermo Fisher Scientific) were applied to the samples for 1 hour at room temperature. Samples were washed 3 times for 5 minutes each with PBS followed by addition of DAPI (1:5000) for 5 minutes. Insert membranes were carefully detached from their transwell support with a fine scalpel, and transferred to a glass microscopic slide. Samples were mounted using Fluoromount G (Thermo Fisher Scientific), imaged on a Zeiss LSM 780 Confocal Microscope and images were processed using Zen Blue software (Zeiss).

### Real Time Quantitative PCR (RT-qPCR)

Total RNA was extracted from mock and SARS-CoV-2 infected proximal airway ALI and AT2 cultures lysed in Trizol (Thermo Fisher Scientific) using the chloroform-iso-propanol-ethanol method. 500 ng of RNA was reversed transcribed into cDNA in a 20 µL reaction volume using iScript cDNA synthesis kit (Biorad) in accordance to manufacturer’s guidelines. RT-qPCR was performed on 10 ng of CDNA per reaction in triplicates for each sample using SYBR green master mix (Thermo Fisher Scientific) on a 7500 Fast Real Time PCR system (Applied Biosystems).

Primers sequences for detection of SARS-CoV-2 N gene were obtained from the Center for Disease Control’s resources for research labs.

### Bulk RNA Sequencing

2 days after SARS-CoV-2 infection, cells were lysed in RLT buffer and total RNA was extracted for bulk RNA sequencing using a Qiagen RNEasy Mini Kit. RNA quality was analyzed using the 2100 Bioanalyzer (Agilent Technologies) and quantified using QubitTM (ThermoFisher Scientific). Library construction was performed by the Cedars-Sinai Applied Genomics, Computation and Translational Core using the Lexogen RiboCop rRNA Depletion kit and Swift Biosciences RNA Library Sciences. Sequencing was performed using the NovaSeq 6000 (Illumina) with single-end 75 bp sequencing chemistry. On average, about 20 million reads were generated from each sample. Raw reads were aligned using Star aligner 2.6.1 (Dobin et al., 2013)/RSEM 1.2.28 (Li and Dewey, 2011) with default parameters, using a custom human GRCh38 transcriptome reference downloaded from www.gencodegenes.org, containing all protein coding and long non-coding RNA genes based on human GENCODE version 33 annotation with SARS-Cov2 virus genome MT246667.1 www.ncbi.nlm.nih.gov/nuccore/MT246667.1.

Differential gene expression was determined by DESeq2 (Love et al., 2014). Top differential genes were determined based on fold change and test statistics. Pathway analysis was performed using Ingenuity Pathway Analysis.

### Statistics

Statistical analysis was performed using GraphPad Prism version 8 software. Data is presented as linear fold change or log2 fold change ± SEM. SARS-CoV-2 infection time course for ALI and AT2 cultures was analyzed using Two- Way ANOVA with Sidak’s post hoc correction. Drug validation experiments were analyzed using One-way ANOVA with Tukey’s post hoc correction. N numbers and p-values are indicated in corresponding figure legends. TPM counts and relative gene expression for ISGs between MOCK and infected cultures were analyzed using Two-tailed t-tests. *p<0.5, **p<0.01, ***p<0.001, ****p<0.0001.

### Results

### SARS-CoV-2 Infects and Replicates Within Human Proximal Airway Epithelial Cells

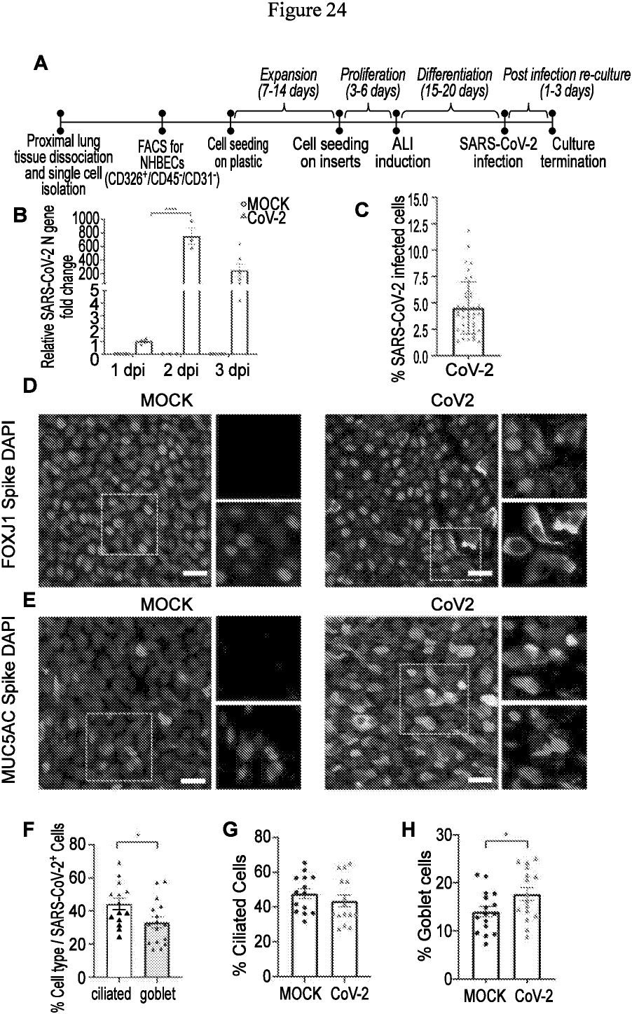

Since the upper respiratory tract represents the most likely initial site of respiratory virus infection, we initially utilized the well-established ALI culture system to study the effect of SARS-CoV-2 infection on proximal airway epithelial cells. Human tracheo-bronchial epithelial cells (HBECs) isolated from trachea and upper bronchi were cultured at ALI for 16-20 days to yield a well differentiated, pseudostratified mucociliary epithelium with approximately 1×105 cells per insert, of which 47% (±10.69%) were ciliated cells and 14% (±5.3%) were goblet cells. ALI cultures of proximal airway epithelial cells were infected apically with SARS-CoV-2 (1×104 TCID50 per well) and harvested for analysis at 1 to 3 days post infection (dpi) (FIG. 24A). SARS-CoV-2 readily infected the well-differentiated proximal airway cells leading to viral replication and gene expression as indicated by induction of SARS-CoV-2 Nucleoprotein gene (N gene) RNA in infected samples. N gene abundance, reflective of either viral mRNA expression or a result of genome replication increased 750-fold at 2 dpi compared to 1 dpi and declined at 3 dpi, however, the later decline was not statistically significant. Viral N gene expression was undetectable in corresponding mock cultures at all 3 time points (FIG. 24B).

Further analysis of proximal airway ALI cultures at 2 dpi revealed that on an average, 4.5% of total cells were infected by SARS-CoV-2 (FIG. 24C). The infection was heterogenous; both ciliated and goblet cells were infected, evidenced by colocalization of SARS-CoV-2 viral capsid “spike” protein with the ciliated cell marker, FOXJ1 (FIG. 24D) and the goblet cell marker, MUC5AC (FIG. 24E). Ciliated cells were infected at a significantly higher rate than goblet cells. Of the total infected cells, 44.2% were ciliated and 33.1% were goblet cells (FIG. 24F). We further assessed whether SARS-CoV-2 infection triggered a change in the proportions of the two cell types. We found no change in the percentage of ciliated cells after SARS-CoV-2 infection (FIG. 24G). However, the proportion of Muc5ac producing goblet cells significantly increased post infection 10 (FIG. 24H).

### SARS-CoV-2 Infects and Replicates Within Human AT2 Cells

Although infection initiates in the proximal airways, a number of recent studies have shown that in severe cases of COVID-19, acute respiratory distress syndrome (ARDS) results due to viral infection of AT2 cells in the distal lung epithelium (Hou et al., 2020, Ziegler et al., 2020). As such, physiologically relevant alveolar infection models including both AT1 and AT2 cells are needed to study the response of alveolar epithelial cells to SARS-CoV-2.

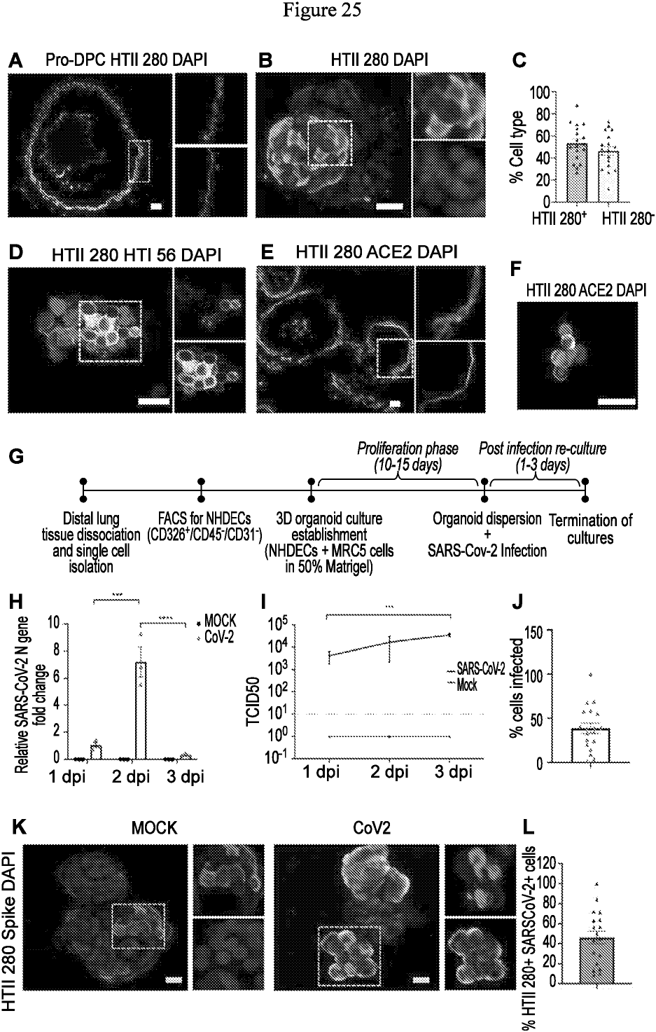

We previously published the development 1 of the first 3D spheroid culture model of the human alveolar epithelium by co-culturing primary epithelial cells isolated from normal human distal lung tissue in the presence of stromal support cells, embedded in Matrigel®. Alveolar spheroid models were further refined to allow culture of AT2 cells in serum free, chemically defined conditions. Our method enabled culture of 3 dimensional alveospheres composed of self-renewing AT2 cells which express the well-established AT2 cell markers, HTII-280 and surfactant protein C (SPC) (FIGS. 25A and 25B). In addition, our cultures also showed presence of some HTII-280 negative cells which on further analysis were found to express the AT1 cell marker HTI 56 (FIGS. 25C and 25D), indicating AT2 to AT1 cell differentiation capacity. We further assessed expression of the SARS-CoV-2 entry receptor, ACE2 in our alveosphere cultures found ACE2 staining localized to HTII-280+ AT2 cells (FIGS. 25E and 25F). This observation is consistent with previous reports of single cell transcriptomic and immunofluorescence analysis of ACE2 expression in human lung.

We went on to utilize our alveosphere system to model SARS-CoV-2 infection of AT2 cells in vitro. Briefly, isolated HTII-280+ AT2 cells were cultured in Matrigel® for 10-15 days to form 3D alveospheres which were then enzymatically released from Matrigel®, followed by gently “opening” alveospheres to allow viral access to the apical membrane by pipetting 3 times with a P1000 tip (Hereafter referred to as AT2 cultures). We then exposed AT2 cultures to SARS-CoV-2 (1×10⁴ TCID50 per well) or PBS (as MOCK controls) and harvested cells for analysis 1-3 dpi (FIG. 25G). Study of viral infection kinetics from 1 dpi to 3 dpi demonstrated that viral N gene expression in cell lysates peaked at 2 dpi. N gene expression at 2 dpi was 7-fold higher compared to the mean infection at 1 dpi and declined significantly by 3 dpi. Viral N gene expression was below the detection limits in corresponding mock cultures at all 1 3 time points (FIG. 25H). In parallel, we also assessed the viral load present in the supernatant medium. Viral load increased with each day in culture, peaking at 3 dpi (FIG. 25I). Differences in the viral N gene expression in cell lysates versus viral loads in supernatant medium at 3 dpi likely reflect increased cell death and virus release at the 3 dpi recovery time point. We used immunofluorescence confocal microscopy to verify SARS-CoV-2 infection of epithelial cells in cultures. The efficiency of infection was significantly higher in AT2 cultures compared to proximal airway ALI cultures, with SARS-CoV-2 infecting 38.6% and 4.5% of total cells in distal and proximal cultures, respectively (FIGS. 25J and 24C). Infection of AT2 cells was confirmed by co-localization of the viral spike protein with HTII-280+ in 2 dpi cultures (FIG. 25K). Of the total HTII 280+ AT2 cells, 46% were infected by SARS-CoV-2 (FIG. 25L).

### SARS-CoV-2 Infection of AT2 Cells Induces a Robust Pro-Inflammatory Response

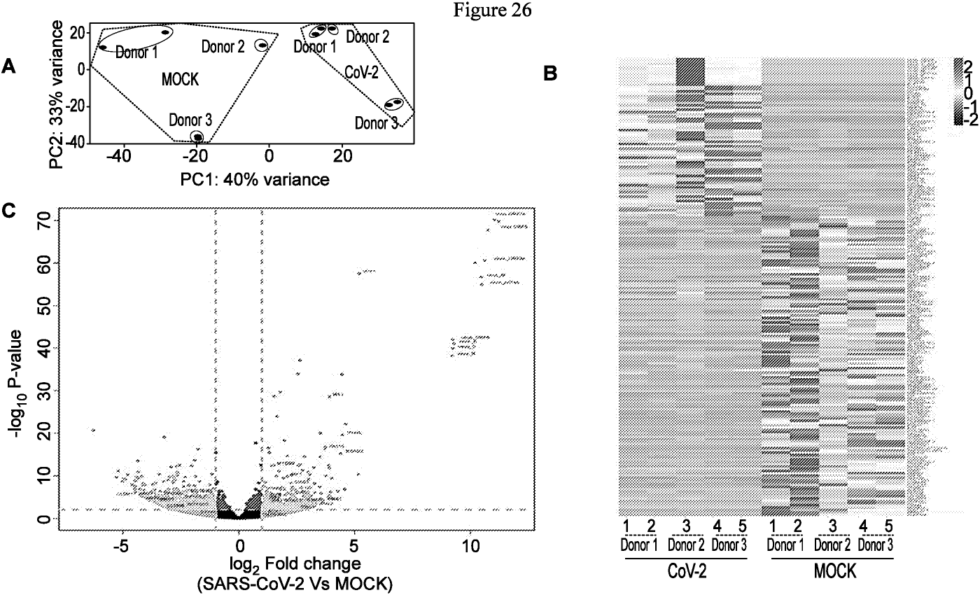

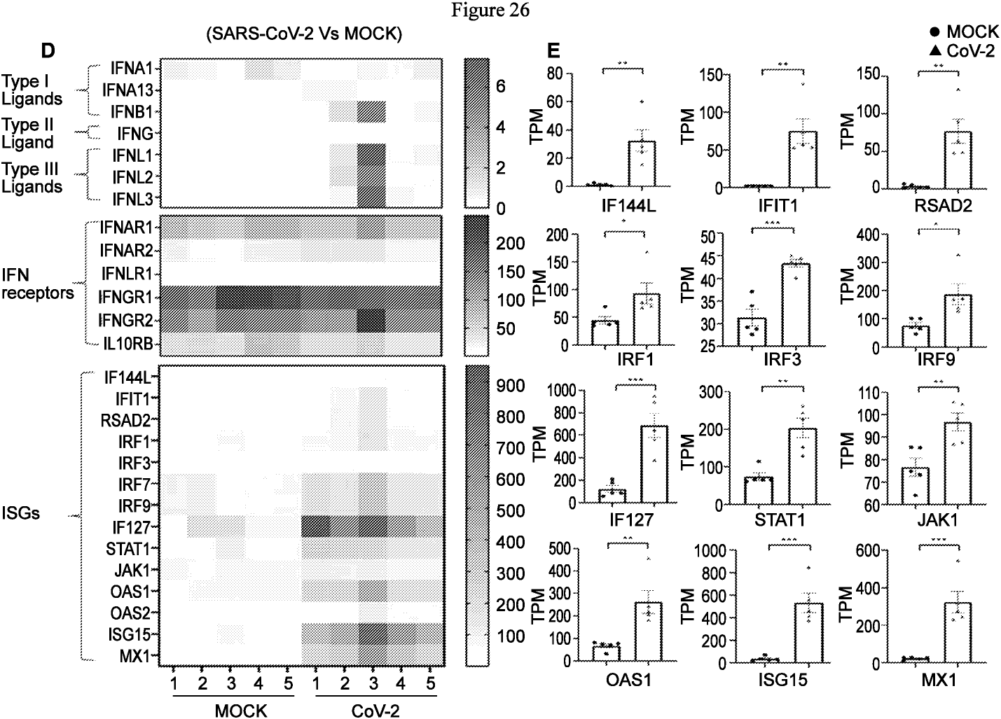

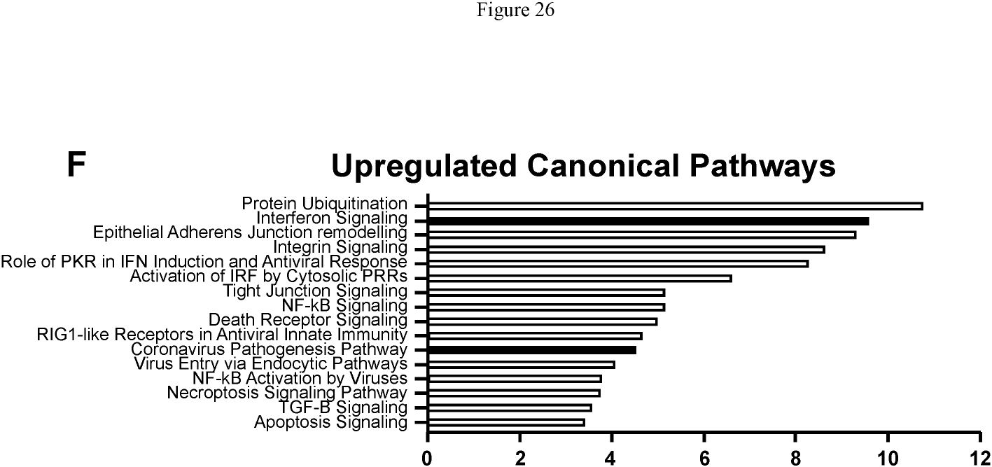

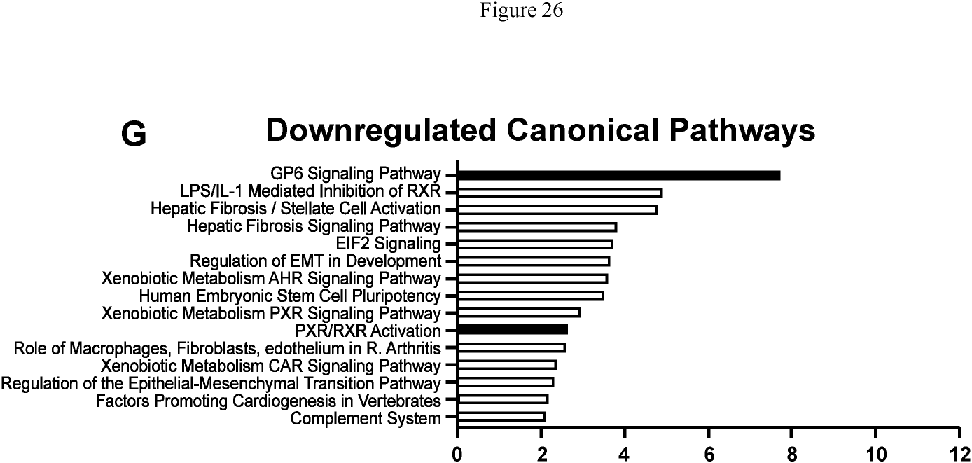

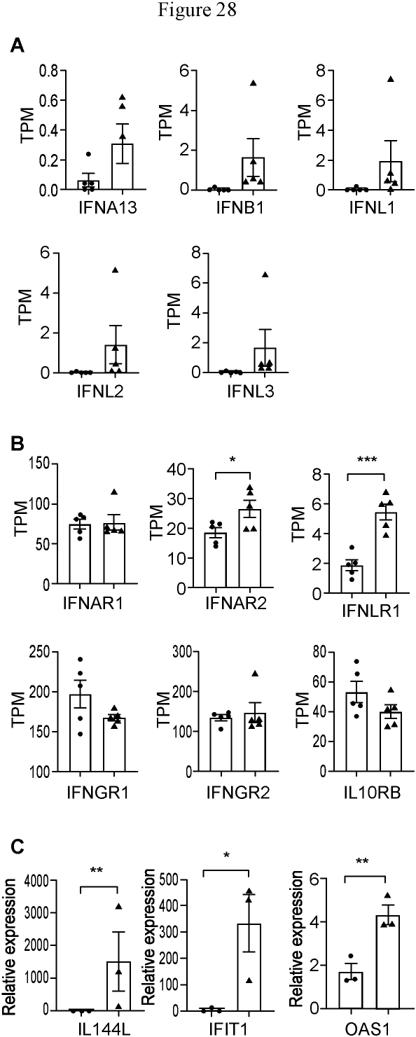

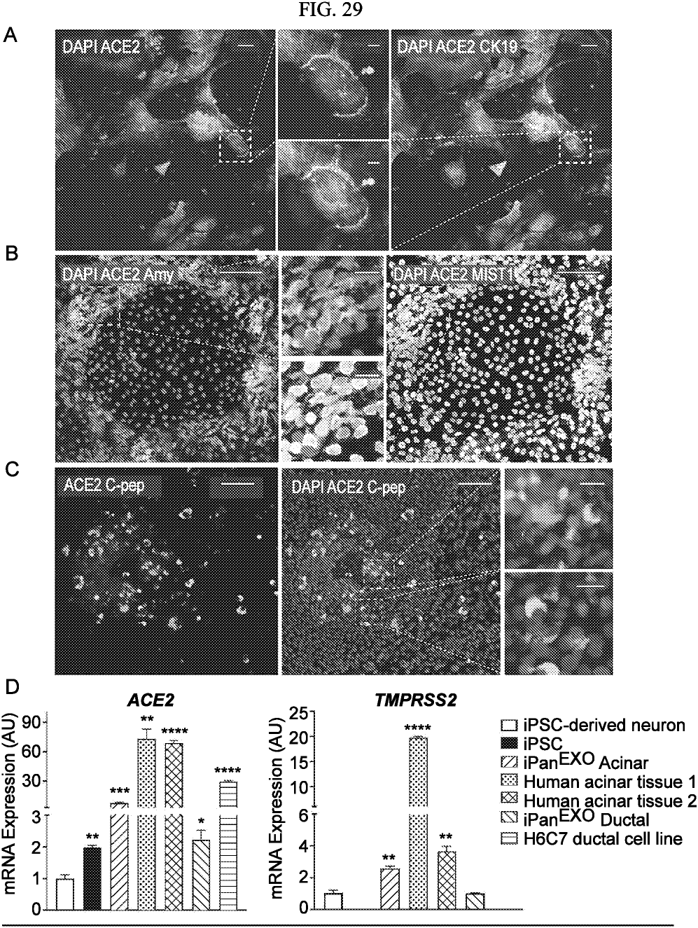

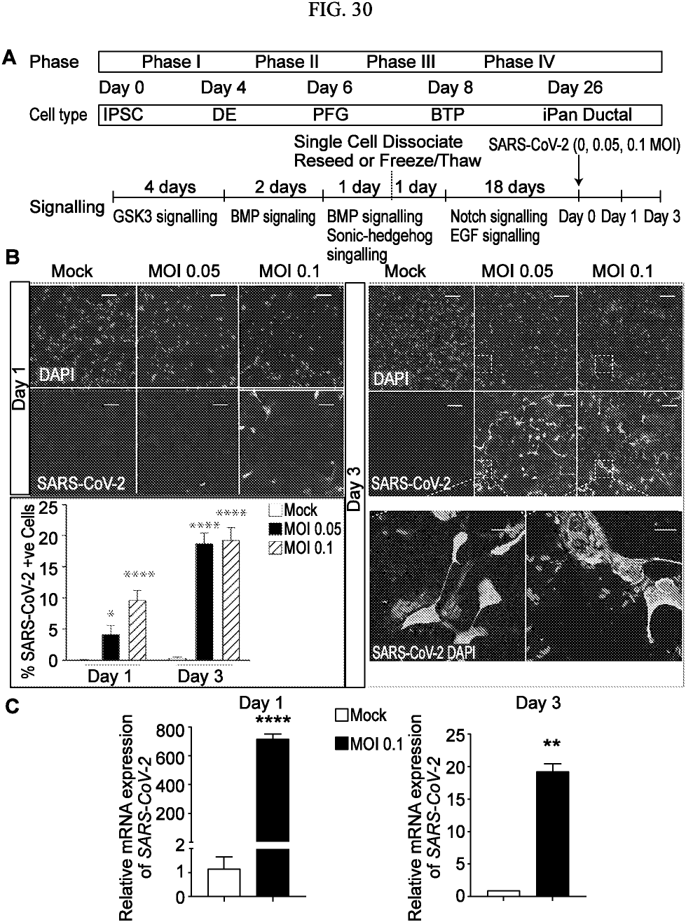

Having established a model that enables robust infection of AT2 cells by SARS-CoV-2, we further evaluated the utility of this system to study host pathogen responses. AT2 cultures were infected with SARS-CoV-2 and cells were harvested 2 dpi for transcriptomic analysis via global RNA-Sequencing (5 samples overall; of which 3 donor cell preparations were represented for both MOCK and SARS-CoV-2 infected cultures). Significant changes in transcript profiles were observed between SARS-CoV-2 infected and mock AT2 cultures (FIG. 26A). Heatmap (FIG. 26B) and volcano plots (FIG. 26C) of differentially expressed genes revealed high levels of SARS-2 CoV-2 viral RNA, such as coding sequences for Virus_N, virus_ORF1ab, virus_ORF3a, 3 virus_ORF7a, further confirming SARS-CoV-2 genome replication and/or gene transcription in AT2 cultures. Infected cultures showed induction of a number of proinflammatory transcripts related to viral infections including type 1 and type 3 interferon related genes and their downstream targets. The type 1 interferon ligands IFNA13 and IFNB1 and type 3 interferon ligands, IFNL1, IFNL2 and IFNL3 were upregulated in the infected cultures (FIG. 26D), although their cumulative changes were not statistically significant (FIGS. 26D and 28A). Our AT2 cultures also expressed interferon receptor transcripts irrespective of viral infection status, with IFNAR2 and IFNLR1 being significantly upregulated in infected cultures compared to mock infected controls (FIGS. 26D and 28B). Donor variability was seen in magnitude of infection between cultures, wherein a direct correlation existed with induction of IFN-related ligand and receptor gene expression.

Additionally, multiple downstream interferon signaling genes (ISGs) such as IF144L, IFIT1, RSAD2 IRF1, IRF3, IRF7, IRF9 IF127, STAT1, JAK1, OAS1, OAS2 ISG15 and MX1 were significantly upregulated in infected cultures (FIGS. 26D and 26E). These observed increases in gene transcript levels were further validated by qRT-PCR (FIG. 28C).

Analysis of differentially regulated canonical pathways revealed that other proinflammatory and viral-sensing pathways, such as NF-kB activation, TGFβ signaling and RIG1-like receptor signaling, were also up-regulated in virus-infected cells (FIG. 26F). On the other hand, various signaling pathways related to regulation of stem cell activation and fate, such as Notch, Wnt and BMP signaling, were downregulated (FIGS. 26C and 26F). However, we did not observe significant changes in expression of the AT2 cell markers including SFTPC, SFTPA1, SFTPA2 and SFTPD (FIG. 26C). Furthermore, we did not observe a change in transcript levels for viral entry genes such as ACE2 and TMPRSS2 (FIG. 26C). Taken together, these data indicate that SARS-CoV-2 infection induces significant upregulation of innate immune response genes in alveolar cells without the participation of recruited immune cells.

### SARS-CoV-2 Infection of AT2 Triggers Cellular Apoptosis

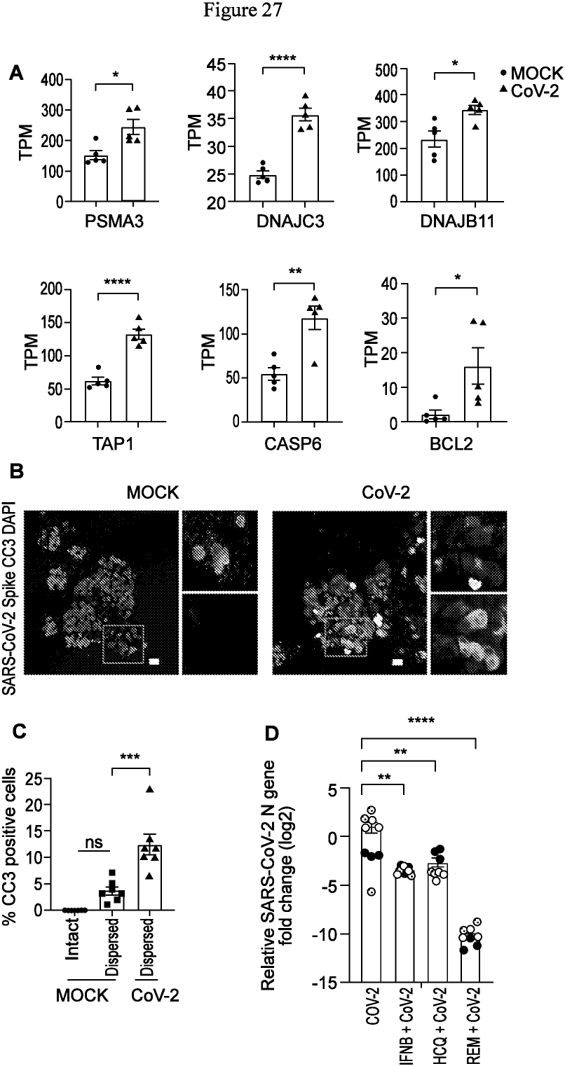

Another key pathway that was activated in SARS-CoV2-infected AT2 cultures included the protein ubiquitination pathway. In particular, we saw a significant increase in transcripts for genes whose products act either as chaperons/co-chaperons or sensitize cells to apoptosis. For example, we saw a significant upregulation PSMA3, DNAJC3, DNAB11 and TAP1 (FIG. 27A), all of which are typically upregulated by cellular stress and interact with key components of apoptotic pathways. Furthermore, we also observed upregulation of apoptosis related genes CASP6 and BCL2 in SARS-CoV-2 infected cultures (FIG. 27A). In order to confirm increased cellular apoptosis in SARS-CoV2-infected cultures, we performed immunofluorescent staining of MOCK and infected cultures for the apoptotic marker, cleaved caspase 3 (CC3). By 3dpi, infected cultures exhibited significantly enhanced staining for CC3 compared 1 to mock cultures. Interestingly only a proportion of the apoptotic cells were infected, thus suggesting that SARS-CoV-2 infection led to direct and indirect cytopathic effect on cultured alveolar epithelial cells (FIGS. 27B and 27C).

SARS-CoV-2 induced apoptosis of neighboring uninfected epithelial cells suggests the potential for a non-cell autonomous effects of viral infection on alveolar epithelial integrity. Together, our data suggest that SARS-CoV-2 infection triggers both cell-autonomous and non-cell-autonomous apoptosis that may contribute to alveolar injury. We conclude that cultures of primary alveolar epithelial cells serve as a robust model for studying the effect of SARS-CoV-2 infection on adult distal lung alveolar epithelium.

### AT2 Cultures as a Tool to Screen Therapeutic Targets Against SARS-CoV-2 Infection

Finally, we utilized the distal AT2 cultures to study the effect of a selected panel of drugs against SARS-CoV-2 infection and replication, including the known anti-viral cytokine, IFNB1 and investigational drugs for COVID-19 treatment, Remdesivir and Hydroxychloroquine. Treatment of AT2 cultures with IFNB1 lead to a statistically significant, 3.2-log reduction in viral N gene RNA compared to control infected AT2 cultures (FIG. 27D). Hydroxychloroquine led to an overall 2.4-log reduction in viral N gene expression compared to average infection in untreated AT2 cultures (FIG. 27D).

However, variable effects of hydroxychloroquine were observed on viral replication/gene expression that were donor epithelium-dependent (FIG. 23). Remdesivir showed the strongest effect on viral replication in AT2 cultures, resulting in a 9-log decrease in viral N gene expression compared to the average infection in untreated cultures (FIG. 27D). This effect was consistent irrespective of donor origin of epithelial cells confirming it as a direct acting antiviral (DAA) agent targeting viral specific RNA polymerase (FIG. 23). 1 Taken together, our data show that our model of primary human AT2 cultures represent a highly relevant preclinical tool to assess SARS-CoV-2 infection and replication, and serves as a sensitive platform for drug screening and validation.

### Example 16

### Ethics Statement and Institutional Approvals

Human cell lines, tissues and histology specimens were obtained or created at Cedars-Sinai under the auspices of the Cedars-Sinai Medical Center Institutional Review Board (IRB) approved protocols. Specifically, the iPSC cell lines and differentiation protocols in the present study were carried out in accordance with the guidelines approved by Stem Cell Research Oversight committee (SCRO) and IRB, under the auspices of IRB-SCRO Protocols Pro00032834 (iPSC Core Repository and Stem Cell Program) and Pro00036896 (Sareen Stem Cell Program). Infections of iPSC-derived cells were performed under the auspices of UCLA’s Stem Cell Oversight Committee under protocol #2020-004-01 and UCLA Biosafety Committee protocol BUA-2020-015-004-A. Post-mortem tissues were collected by Cedars-Sinai’s Biobank and Translational Research core in accordance with protocol Pro00036514.

### Human iPSC Culture

The induced pluripotent stem cell (iPSC) lines utilized in this study were generated from healthy volunteers at the iPSC Core at Cedars-Sinai Medical Center from the peripheral blood mononuclear cells (PMBCs) utilizing non-integrating oriP/EBNA1-based episomal plasmid vectors, as described in (Rajamani et al., 2018). This approach results in highly cytogenetically stable iPSCs as tested by G-band karyotyping. All undifferentiated iPSCs were maintained in mTeSR⁺ media (StemCell Technologies, Cat 05825) onto BD Matrigel™ matrix-coated plates.

### Generation of iPSC-Differentiated Pancreatic Progenitors

iPSCs were single-cell dissociated using Accutase and plated onto Matrigel-coated plates at a density of 300.000 cells/cm² using mTeSR⁺ and 10 µM Rho kinase Inhibitor (Stem Cell Tech). The following day, cells were directed into Definitive Endoderm (DE) using Phase I medium, which was composed of base medium MCDB 131 (Fisher Sci) supplemented with 100 ng/ml Activin A (R&D), 2 µM CHIR99021 (Stemgent), and 10 µM Rho kinase Inhibitor (Stem Cell Tech.) for 1 day. For the next two days, the same base medium was used, but supplemented instead with 100 ng/mL Activin A and 5 ng/mL FGF2 (Peprotech). Following this phase, cells were directed to form Posterior Foregut (PFG) using Phase II medium, which was composed of the same base medium as Phase I but supplemented with 50 ng/mL FGF10 (Peprotech), 0.25 µM CHIR99021 and 50 ng/ml Noggin (Peprotech), for 2 days. To reach a Pancreatic Progenitor (PP) stage, cells were fed with Phase III medium, which was composed of DMEM supplemented with 50 ng/mL Noggin, 50 ng/mL FGF10, 2 µM Retinoic Acid (Sigma), and 0.25 µM SANT1 (Sigma), for four days.

### Generation of iPSC-Differentiated Pancreatic Endocrine (iPan^{ENDO}), Acinar (iPan^{EXO} Acinar) and Ductal (iPan^{EXO} Ductal) Cells

To generate iPan^{EXO} Ductal cells, on Day 6 of differentiation, cells were single cell dissociated and seeded at 103.1 k cells/cm² in Phase III medium supplemented with 10 µM Rho kinase Inhibitor on a Matrigel-coated plate. From Day 7 to Day 22, cells were grown in iPan^{EXO} Ductal phase media, which consisted of Phase III base medium supplemented with 25 ng/ml FGF10, 50 ng/ml EGF (Peprotech), and 50 ng/ml sDLL-1 (Peprotech). To generate iPan^{ENDO} and iPan^{EXO} Acinar cells, after 4 days of Phase III, PPs were fed with Phase III base medium supplemented with 20 ng/mL FGF10, 1 µM XXI (Sigma Aldrich), 50 ng/mL Noggin, 10 mM Nicotinamide (Sigma-Aldrich), and 25 ng/mL Wnt3a (Peprotech), for seven days, daily feeding.

Phase I & II base medium

| Component | Amount | Final concentration |
|---|---|---|
| MCDB131 (FisherScientific) | 98 ml | |
| Glutamax (FisherScientific) | 1 ml | 2 mM |
| Vitamin C (Sigma) | 100 µl | 250 µM |
| BSA (VWR) | 500 mg | 0.5% |
| NaHCO3 (Sigma) | 150 mg | 1.5 g/L |
| Pen/Strep (Sigma) | 1 ml | 1% |
| Phase III base medium | | |
| Component | Amount | Final concentration |
| DMEM (ThermoFisher) | 98 ml | |
| Vitamin C | 100 µl | 250 µM |
| B27 supplement without Vitamin A (ThermoFisher) | 100 µl | 1% |
| Pen/Strep | 1 ml | 1% |

### Generation and Maintenance of iPan^{EXO} Organoid Cultures

On the last day of Phase III, PPs were roughly dissociated via scraping and trituration. They were centrifuged at 170 × G for 3 minutes, and then resuspended with a solution composed of Phase III medium and Matrigel® at a 1:4 ratio (1 of medium and 4 of Matrigel®). 30 µL of this solution with cells was plated into each well of a 96 round bottom plate and then incubated at 37° C. for 20 minutes, before the plate was flipped upside down for 10 minutes. 100 µL of Phase III base medium supplemented with 20 ng/mL FGF10, 50 ng/mL Noggin, 10 mM Nicotinamide, and 25 ng/mL Wnt3a was then added to the cells. Cells were fed every other day with the same media until Day 57 or when organoids contained lumen and were at least 150 µm large.

### SARS-CoV-2 Stock

SARS-CoV-2, isolate USA-WA1/2020, was obtained from the Biodefense and Emerging Infections (BEI) Resources of the National Institute of Allergy and Infectious Diseases (NIAID). Importantly, all studies involving SARS-CoV-2 infection of iPSC-derived pancreatic cells were conducted within a Biosafety Level 3 high containment facility at UCLA. SARS-CoV-2 was passed once in Vero-E6 cells and viral stocks were aliquoted and stored at - 80° C. Virus titer was measured in Vero-E6 cells by TCID50 assay. Vero-E6 cells were cultured in DMEM growth media containing 10% FBS, 2 mM glutamine, pen/step, and 10 mM HEPES. Cells were incubated at 37° C. with 5% CO₂.

### SARS-CoV-2 Infection of iPSC-Derived Pancreatic (iPan) Cultures

SARS-CoV-2 viral inoculum (MOI of 0.05 and 0.1) was prepared using acinar or ductal cell specific media. Human iPSCs were differentiated into iPSC-derived pancreatic (iPan) cultures containing iPan^{ENDO}, iPan^{EXO} Acinar and iPan^{EXO} Ductal cells in 96-well or 24-well plates before infection as detailed above, and the culture media at Day 26 of differentiation for iPan^{EXO} Ductal and Day 16 for iPan^{EXO} Acinar were replaced with 100 µl of prepared inoculum. For Mock infection, cell type specific media (100 µl/well) alone was added. The inoculated plates were incubated for 1 hour at 37° C. with 5% CO₂. At the end of incubation, the inoculum was replaced with fresh iPan^{EXO} Acinar or iPan^{EXO} Ductal culture medium. Cells remained at 37° C. with 5% CO₂for 24 hours or 72 hours before analysis. All studies involving active SARS-CoV-2 infection of iPSC-derived pancreatic cell cultures were conducted within a Biosafety Level 3 facility at University of California in Los Angeles (UCLA), CA, USA.

### Immunofluorescence and Imaging of Cells

Cells subjected to immunofluorescence were fixed with 4% paraformaldehyde (PFA) in phosphate-buffered saline (PBS) for 20 minutes and subsequently washed with PBS. Fixed cells were then permeabilized and blocked for 1 hour in a blocking buffer containing PBS with 10% donkey serum (Millipore) and 0.1% Triton-X (Bio-Rad). Primary antibodies were diluted in the blocking buffer and added to the cells overnight at 4° C. The following primary antibodies and dilutions were used: SARS Coronavirus (1:400, NR-10361, BEI Resources) SARS-CoV-2 Spike S1 (1:100, 40150-R007, SinoBiological), eCAD (1:100, AF68, R&D Systems) , MIST1 (1:100, MA1517, Invitrogen), CK19 (1:100, PIMA512663, Thermofisher), SOX9 (1:250, AB5535, Millipore), ACE2 (1:100, AB15348, abcam), Chymotrypsin (1:100, MAB1476, Millipore), Amylase (1:100, A8273, Sigma), SMA (1:100, Millipore, A2547), CD68 (1:100, R&D Systems, MAB20401), CD31 (1:100, Cell Signaling, 3528). After washing using PBS with 0.1% Tween-20 (ThermoFisher), cells were incubated with appropriate species-specific Alexa Fluor-conjugated secondary antibodies (ThermoFisher) diluted in a blocking buffer (1:1,000) for 1 hour at room temperature. After washing in PBS with 0.1% Tween-20, cells were incubated in Hoechst 33342 diluted in PBS (1:2,500) for 15 min. Immunofluorescence images were visualized using appropriate fluorescent filters using ImageXpress Micro XLS (Molecular Devices) and analyzed using ImageJ Software. Image quantification was performed using CellProfiler Software (v3.1.9).

### Real-Time qPCR

Relative gene expression was quantified using RT-qPCR. For this, cells were washed with PBS and the total RNA was isolated with RLT (Qiagen). RNA was then extracted with RNeasy Micro Kit (Qiagen) according to the manufacturer’s instructions. The concentration of RNA was determined by spectrophotometric analysis (Qubit 4 Fluorometer, ThermoFisher) and the purity with NanoDrop (ThermoFisher); all samples had a A_{260/280} ratio around 2.0 (Desjardins and Conklin, 2010). After, RNA (1 µg) was reverse transcribed to cDNA with oligo(dT) using the High Capacity cDNA Reverse Transcription kit (ThermoFisher). Real-time qPCR was performed in triplicates using SsoAdvanced Universal SYBR Green Supermix (Biorad) and specific primer sequences to each gene, on a CFX384 Real Time system (Bio-Rad). Human RPL13 was used as the reference gene and relative expression was determined using 2^{-ΔΔ} Ct method.

### Post-Mortem Human Pancreatic Tissues

a) Real-Time qPCR from snap-frozen tissues. Post-mortem pancreatic samples were isolated from the head of the pancreas (preferably) from patients that were infected with SARS-CoV-2 and passed from complications related to the disease (COVID-19 patients), or patients that were not infected with SARS-CoV-2 and passed from complications not related to it (Control patients). These samples were isolated 1-3 days after the patient’s death and were snap-frozen in liquid nitrogen. These samples were stored for longer in -80° C. before used for either RNA extraction or immunohistochemistry. For RNA isolation, Trizol (Thermo) was used and for RNA extraction, RNeasy Mini Kit (Qiagen) was used according to the manufacturer’s instructions. The concentration of RNA was determined by spectrophotometric analysis (Qubit 4 Fluorometer) and the purity with NanoDrop, as described above; all samples had a A_{260/280} ratio around 2.0. After, RNA (2 µg) was reverse transcribed to cDNA with oligo(dT) using the High Capacity cDNA Reverse Transcription kit. Real-time qPCR was performed in triplicates using SsoAdvanced Universal SYBR Green Supermix (Bio-Rad). qPCR was performed on a CFX384 Real Time system. Human β-ACTIN was used as the reference gene and relative expression was determined using 2^{-ΔΔ} Ct method.

b) Immunohistochemistry from paraffin embedded tissues. A different set of COVID-19 patients were utilized for immunohistochemistry. For this, pancreatic tissues were fixed in 10% formalin and then paraffin embedded. Blocks were sectioned at 4 µm thickness and mounted at microscope slides (Superfrost Plus microscope slides, Fisher). Slides were washed 2x in Xylene for 10 minutes each. This was followed by 2x 5-minute washes with 100% ethyl alcohol, 1x 3-minute wash with 95% ethyl alcohol, and 1x 3 minute wash with 75% alcohol for rehydration. They were then washed 3x with PBS for 5 minutes before beginning antigen retrieval. Samples were submerged in 10 mM pH 6.0 sodium citrate Buffer, and then microwaved for 10 minutes at 80% power. Once cooled at room temperature for one hour, they were washed 3x for 5 minutes each with PBS. Samples were blocked for 2 hours in the same blocking buffer as mentioned above (PBS with 10% donkey serum and 0.1% Triton-X). Samples were incubated 4° C. overnight with primary antibodies in the same concentrations as mentioned above. The following day, they were washed 3x for 10 minutes each in PBS, incubated one hour at room temperature with the secondary antibodies at concentrations of 1:1000, and then finally washed 3x for 10 minutes each before mounted with ProLong™ Gold Antifade Mountant with DAPI (Invitrogen).

### RNA Sequencing (RNASeq)

a) RNA extraction and sequencing. RNA was extracted from pelleted cells using RNeasy Micro Kit (Qiagen) and was prepared for sequencing with the Illumina TruSeq Stranded mRNA library preparation kit (Illumina, San Diego, CA) by the Cedars-Sinai Applied Genomics, Computation, and Translational Core. Concentration and quality of RNA was assessed on a Qubit fluorometer (ThermoFisher Scientific, Waltham, MA) and 2100 Bioanalyzer (Agilent Technologies, Santa Clara, CA) respectively. Complementary DNA was reverse transcribed using Invitrogen’s Reverse Transcriptase kit (Carlsbad, CA) and converted into double-stranded DNA (dsDNA). The dsDNA was then enriched using PCR before purification with Agencourt AMPure XP beads (Beckman Coulter, Brea, CA). The enriched purified DNA was then quantified and resolved via Qubit and Bioanalyzer. Sample libraries were then multiplexed and sequenced on Illumina’s NextSeq 500 platform (San Diego, CA) using 75 bp single-end sequencing.

b) Analysis. Raw reads were quantified by mapping them with the STAR aligner (version 2.5.0) (Dobin et al., 2013) / RSEM (version 1.2.25) (Li and Dewey, 2011) to the GRCh38 human reference transcriptome based on human GENCODE version 33 (www.gencodegenes.org) as well as the GenBank: MT246667.1 SARS-CoV-2 reference viral genome. Expression tables were post-processed with a custom R script that filtered out non-coding RNA based on the transcript biotype assigned by biomaRt. Initial analyses were performed on the BioJupies platform. Principal Component Analysis was then performed in R using the prcomp package. Differential expression tables were calculated in R using the DESeq2 package. Differential expression tables were used to plot heatmaps and volcano plots in ggplot. Enrichment analysis was performed by uploading the top 500 upregulated and downregulated transcripts to the Enrichr gene enrichment analysis portal (Chen et al., 2013; Kuleshov et al., 2016). Enrichment results of interest (including Gene Ontology enrichment and COVID-related gene enrichment) were exported from Enrichr as text files and imported into R for plotting using ggplot.

### Statistical Analyses

Data are presented as mean ± standard error of the mean (SEM). Statistical significance between groups was determined by One-way ANOVA followed by Dunnet post-test. Two-tailed paired Student’s test was used as appropriate. P values <0.05 were considered statistically significant. Statistical analyses and graphs were generated using GraphPad Prism 7 for Windows Software (GraphPad Software).

### Results

### iPSC-Derived Pancreatic Cells Exhibit ACE2 and TMPRSS2 Expression

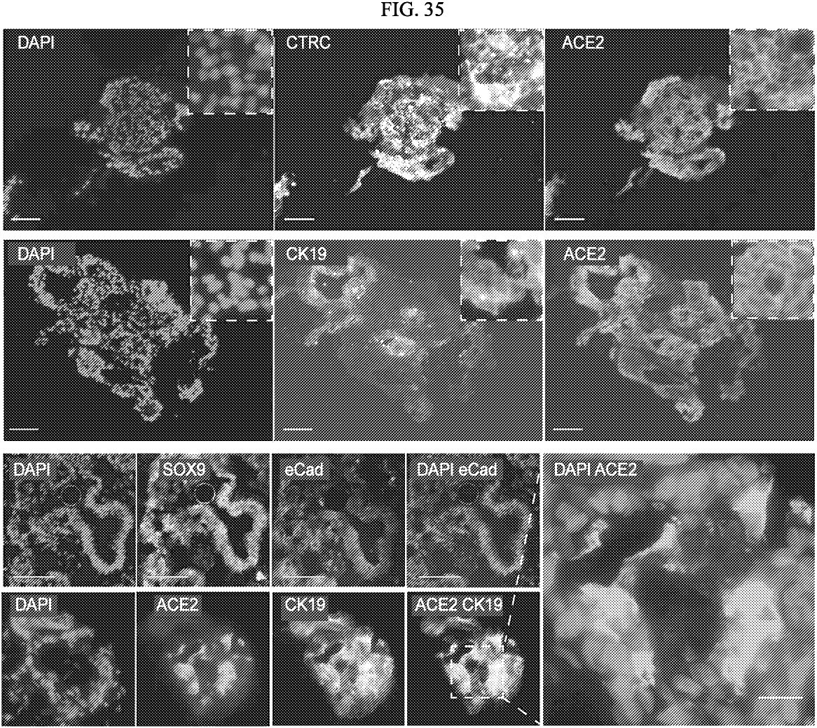

Human iPSCs were differentiated into pancreatic adherent (2D) and organoid (3D) cultures. Protocols for differentiation after the pancreatic progenitor stage were directed to bias the cell fate of the cultures containing either exocrine acinar cells (iPan^{EXO} Acinar) or exocrine ductal cells (iPan^{EXO} Ductal). iPan^{EXO} Acinar cultures express cytoplasmic staining of digestive enzyme Amylase (AMY), digestive enzyme Chymotrypsin C (CTRC), and nuclear transcription factor MIST1, but also contain some C-peptide expressing endocrine β-cells (iPan^{ENDO}), while iPan^{EXO} Ductal cultures express cytoskeletal staining of Cytokeratin 19 (CK19) and nuclear staining of SOX9. Interestingly, both cultures exhibit ACE2 protein expression (FIGS. 29A-C), which is now a confirmed entry point receptor for SARS-CoV-2. ACE2 and TMPRSS2 gene expression was measured in pluripotent iPSC stage cells, iPSC-derived pancreatic cultures, human cadaveric pancreatic tissue, and human pancreatic duct epithelium cell line, H6C7. Relative to iPSCs, iPan^{EXO} Acinar and iPan^{EXO} Ductal cultures contain significantly higher levels of ACE2. There is also a significantly higher expression of TMPRSS2 in iPan^{EXO} Acinar and human acinar tissues, but no detection of this gene in H6C7 cell line and low detection in iPan^{EXO} Ductal cells (FIG. 29D). Differentiated iPan^{EXO} 3D organoids also show co-localization of ACE2 protein along with acinar and ductal markers (FIG. 35).

### SARS-CoV-2 Can Directly Infect iPan^{EXO} Ductal Cultures and Elicit Abnormal Cellular Phenotypes

After 26 days of pancreatic ductal differentiation starting from iPSCs, cultures were infected at a multiplicity of infection (MOI) 0.05 and 0.1 for 24 hours (Day 1) and 72 hours (Day 3) (FIG. 30A). The Mock condition was treated with ductal media with no virus as the control condition. MOI 0.05 condition shows 4% and 19% of cells infected with SARS-CoV-2 on Day 1 and Day 3 respectively, while MOI 0.1 condition shows 10% and 19% of infection, respectively (FIG. 30B). As expected, the Mock condition has negative SARS-CoV-2 staining (FIG. 30B). SARS-CoV-2 mRNA expression was increased on Days 1 and 3 on infected cells compared to Mock condition (FIG. 30C).

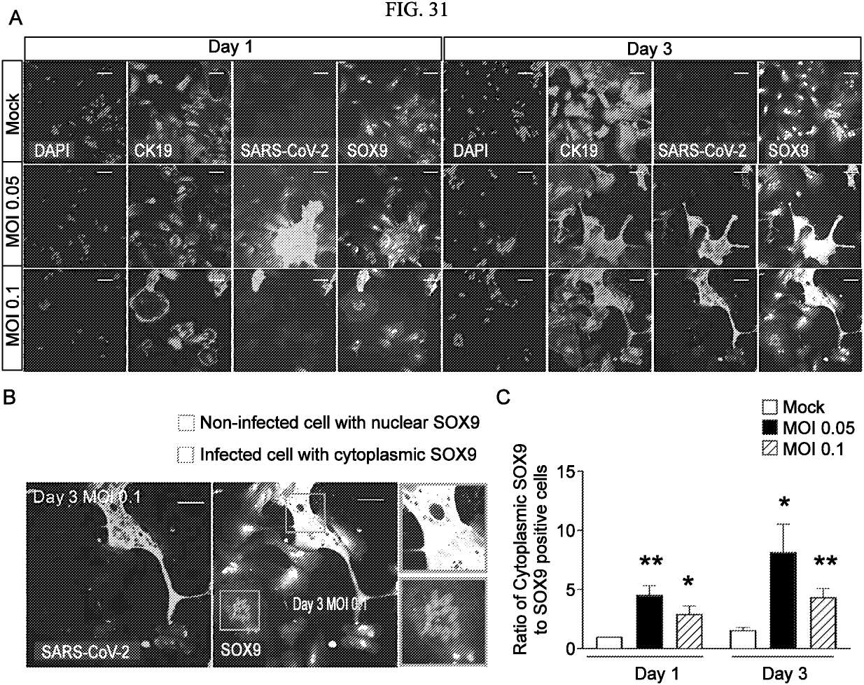

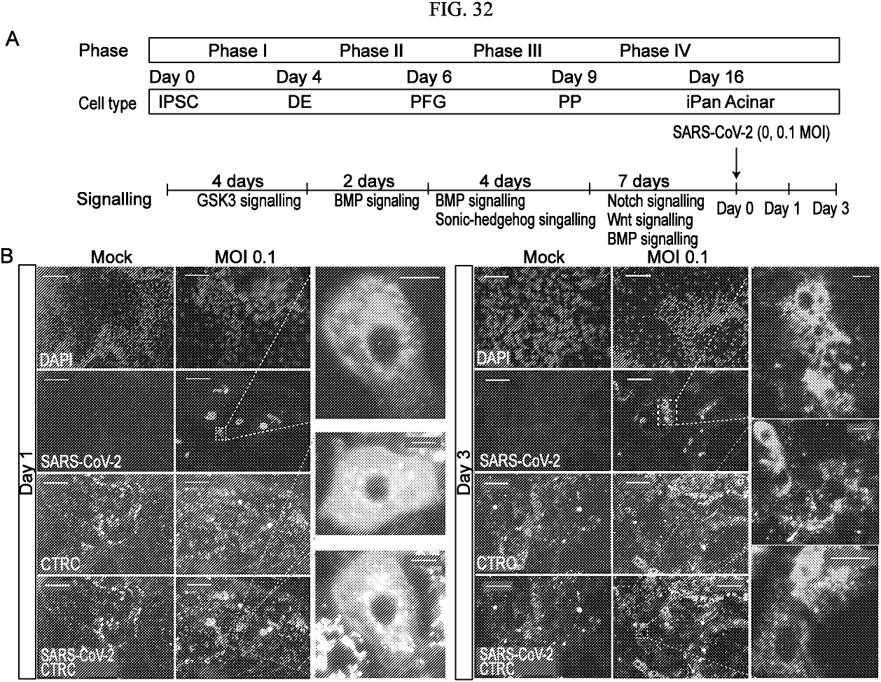

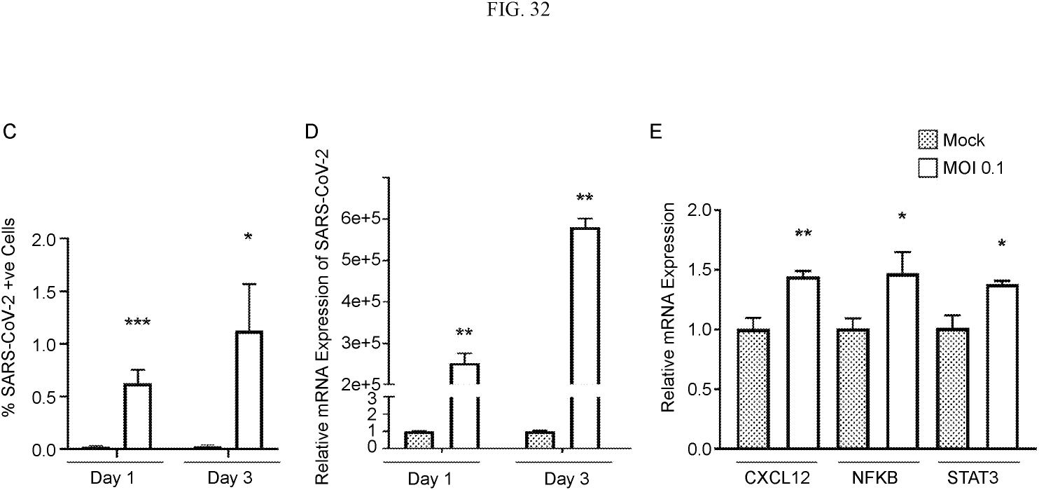

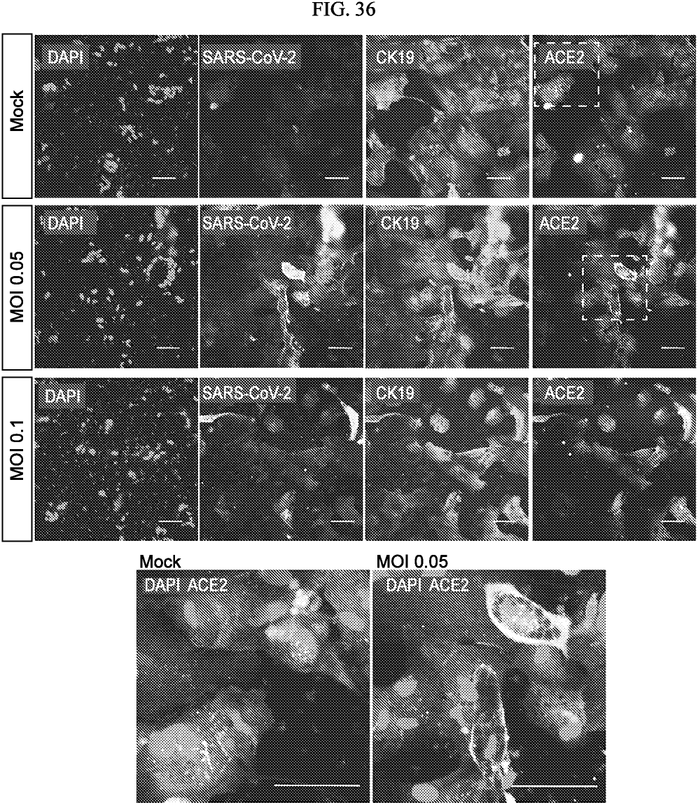

SOX9 is a well-established nuclear transcription factor involved in determining ductal specification from pancreatic progenitors. Interestingly, SARS-CoV-2 positive cells in the infected iPan^{EXO} Ductal cultures at both MOIs show a more enlarged cellular morphology as observed by CK19 and SOX9 staining pattern (FIG. 31A) and abnormal cytoplasmic SOX9 mislocalization instead of the typical nuclear localization observed in normal ductal cells as evident in the mock control condition (FIG. 31B). Compared to the Mock condition there was an increase in ductal cells with mislocalization of SOX9 into the cytoplasm in the infected cultures at 24 hours (Day 1) and 72 hours (Day 3) post SARS-CoV-2 inoculation. At MOI 0.05, there were 5-fold and 8-fold increase in cells with mis-localized cytoplasmic SOX9 at Day 1 and Day 3, respectively (FIG. 31C). At MOI 0.1, there were 3-fold and 4-fold greater cells with cytoplasmic SOX9 compared to Day 1 and Day 3 Mock conditions, respectively (FIG. 31C). Meanwhile, CK19 is present across all treatments and timepoints, with no obvious difference in staining localization of the positively infected population. ACE2 expression is observed in Day 3 cells with no distinct differences between Mock, MOI 0.05 and 0.1 conditions (FIG. 36).

### SARS-CoV-2 Can Infect iPan^{EXO} Acinar and iPan^{ENDO} Cells and Upregulate Proinflammatory Genes

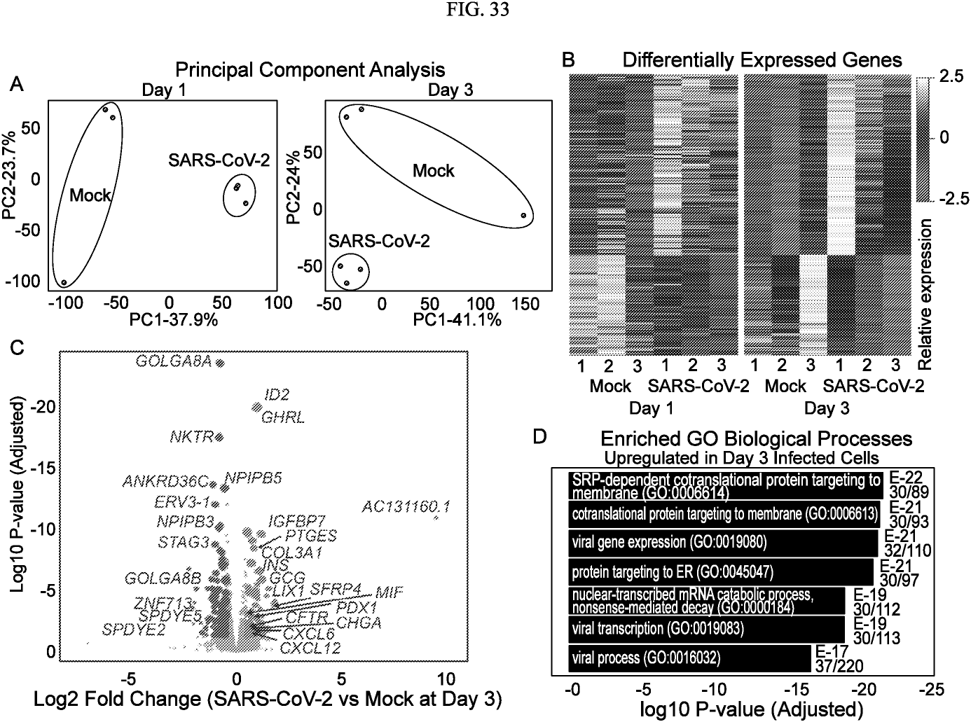

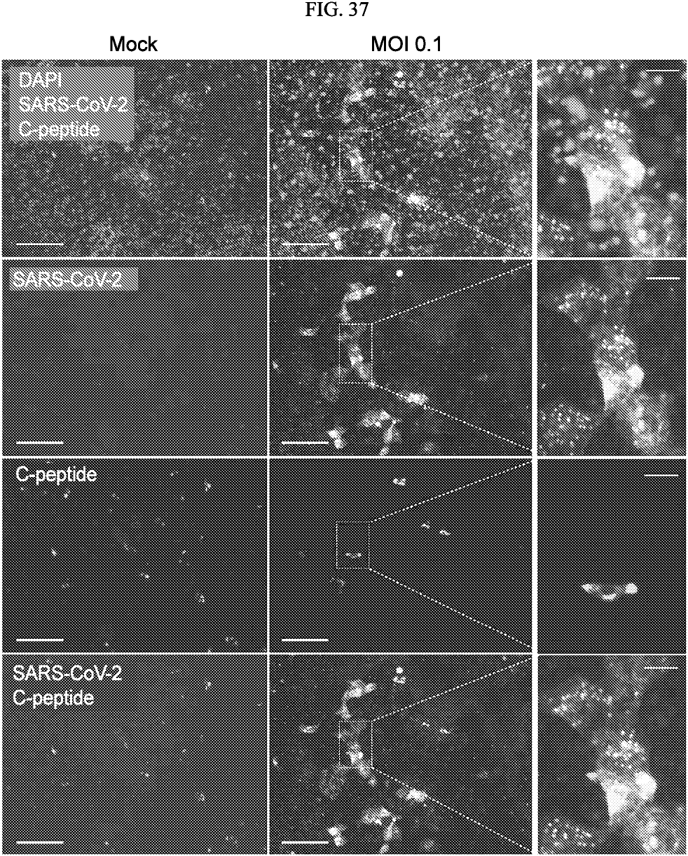

iPan^{EXO} Acinar cultures containing predominantly Chymotrypsin C (CTRC) and Amylase (AMY) positive cells and few iPan^{ENDO} C-peptide positive β-cells were differentiated for 16 days before being infected with SARS-CoV-2 at an MOI of 0.1. A separate Mock condition was cultured in parallel and not infected. Like the iPan^{EXO} Ductal cultures, infected and uninfected cultures were fixed or lysed after 24 hours (Day 1) or 72 hours (Day 3) after the cultures were treated with the virus (FIG. 32A). In comparison to the Mock condition, on both days the infected conditions showed SARS-CoV-2 positive cells, with clear co-staining of SARS-CoV-2 in CTRC-positive cells (FIG. 32B). The CTRC stain has granular morphology that likely reflects zymogen granule formation. A subpopulation of these SARS-CoV-2 positive cells were also C-peptide positive (FIG. 37). On day 1 post-infection, 0.63% of total cells were infected, which increased to 1.12% by Day 3. For both days, the difference in percentage of infected cells was statistically significant compared to Mock (FIG. 32C). These results were confirmed by RT-qPCR for SARS-CoV-2, which showed an approximately 250,000-fold upregulation in the infected cells compared to Mock on Day 1, and an approximately 1,500,000-fold upregulation compared to Mock on Day 3 (FIG. 32D).

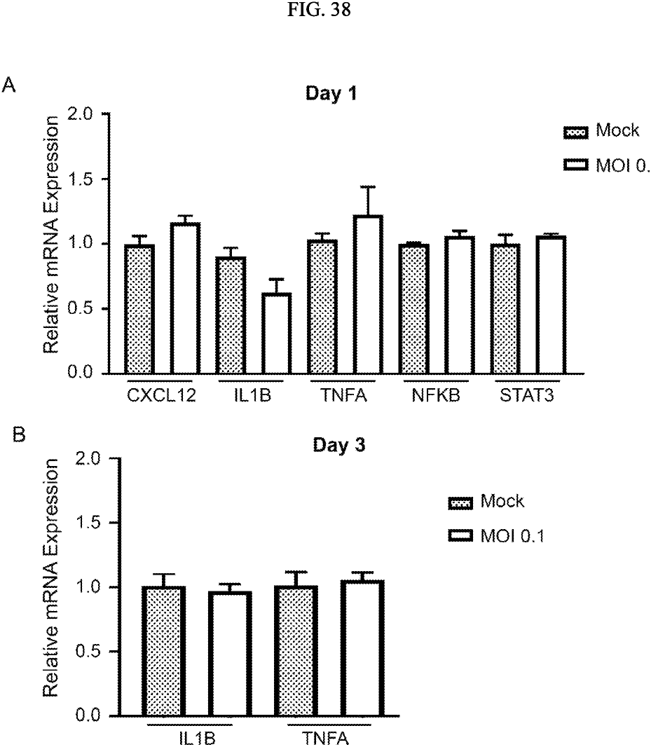

To investigate whether SARS-CoV-2 infection perturbed the inflammatory pathway, multiple genes known to be associated with pancreatitis-related inflammation were assessed using RT-qPCR. Among those, the genes CXCL12, NFKB1, and STAT3 showed significant upregulation at Day 3 in infected cells compared to Mock condition (FIG. 32E). No significant change in expression was seen on Day 1 (FIG. 38). RT-qPCR was also performed for other inflammatory markers, IL1B and TNFA, however, no significant change in expression was seen on either Day 1 or Day 3 (FIG. 38).

### Transcriptional Analysis of SARS-CoV-2 Infected iPSC-Derived Pancreatic Cultures Demonstrates Active Viral Transcription and Pancreas-Specific COVID-19 Associated Disease Signatures

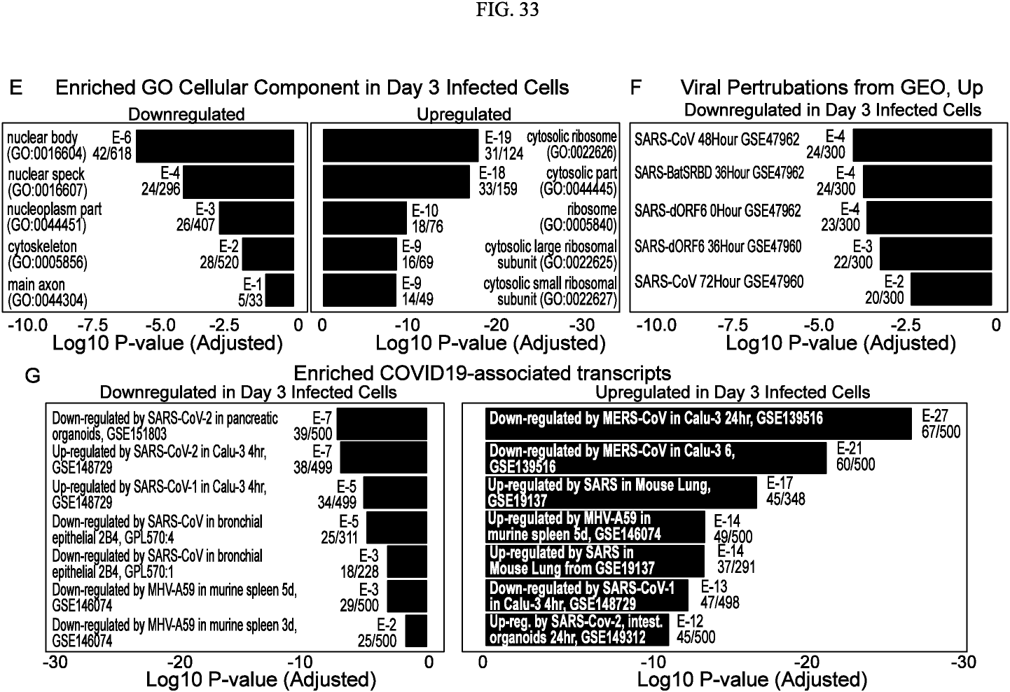

The iPan cultures containing iPan^{EXO} Acinar and iPan^{ENDO} cells and infected with 0.1 MOI SARS-CoV-2 for 24 h (Day 1) and 72 h (Day 3) were also harvested for transcriptomic analysis after mRNA sequencing. After mapping genomic reads in infected cultures, mapped reads were detected from the SARS-CoV-2 genome that confirm active SARS-CoV-2 viral genome replication within infected iPan^{EXO} Acinar cultures. Principal component analysis (PCA) of Day 1 and Day 3 infected cultures demonstrated that 37.9% and 41.1% of the variance in the gene expression differences could be attributed to principal component 1 (PC1) in Day 1 and Day 3 infected cultures, respectively (FIG. 33A). Both PCA and the heatmaps of differentially expressed genes clearly demonstrated transcriptomic clustering of samples in either the Mock or SARS-CoV-2 infected condition in both days (FIG. 33B). Upon further interrogation of the Day 3 data, SARS-CoV-2 infection induced significant pancreas-specific gene expression changes within iPSC-derived pancreatic cultures including upregulation of PDX1, INS, GCG, CHGA, CFTR, IGFBP7 and GHRL and downregulation of genes involved in cellular secretory pathway such as GOLGA8A and GOLGA8B. Significant changes in expression of chemokine and immunomodulatory genes were detected and most were upregulated such as PTGES, MIF, CCR7, CXCL6 and CXCL12, which encodes immune cytokines known to be transcriptionally upregulated during SARS-CoV infection. Similarly, genes reflecting pathogenic interaction and antiviral responses in host cells such as THOC1, TRIM28, CD37, TRAF3IP1, DDX17, NCBP3, HYAL2, were also perturbed. Gene enrichment pathway analysis confirmed significant upregulation of biological processes transcriptional pathways related to signal recognition particle (SRP)-dependent translation and targeting of proteins to the endoplasmic reticulum (ER) membrane, viral transcription and viral gene expression (FIG. 33D), consisting of mainly ribosomal complex large and small subunit genes. This is consistent with the idea that SARS-CoV-2 like many other viruses requires recruitment of a variety of host cell factors including ribosomal proteins to participate in viral protein biosynthesis, in order to survive, accumulate and propagate in the pancreatic cells. The most significant cellular components that are downregulated include nuclear body, nuclear specks and nucleoplasm (FIG. 33E), which is also consistent with the downregulation of the specific nuclear pore complex (NPC) genes such as NPIPB3, 4, 5 (FIG. 33C). Numerous viral pathogens have evolved different mechanisms to hijack the NPC in order to regulate trafficking of viral proteins, genomes and even capsids into and out of the nucleus thus promoting virus replication (Le Sage and Mouland, 2013). As expected, the cytosolic ribosomal units are the most significantly upregulated cellular component (FIG. 33E). Notably, the virus perturbation signatures with SARS-CoV-2 in iPSC-derived pancreatic cells were similar to responses by previously published SARS-CoV, SARS-dORF6 and SARS-BatSRBD infection of human lung alveolar epithelium cells (FIG. 33F) (Mitchell et al., 2013). Upon comparing COVID-19 associated transcripts published and available in the Enrichr database, similar signatures were significantly perturbed in SARS-CoV-2-infected iPan^{Exo} Acinar, consistent with prior reports examining viral infection in pancreas (FIG. 33G). Taken together, these results indicate that SARS-CoV- 2 infection induces significant transcriptional changes within iPSC-derived pancreatic cultures.

### Post-Mortem Human Pancreatic Tissues From COVID-19 Patients Show Infectivity and Perturbed Expression of Pancreatic Genes

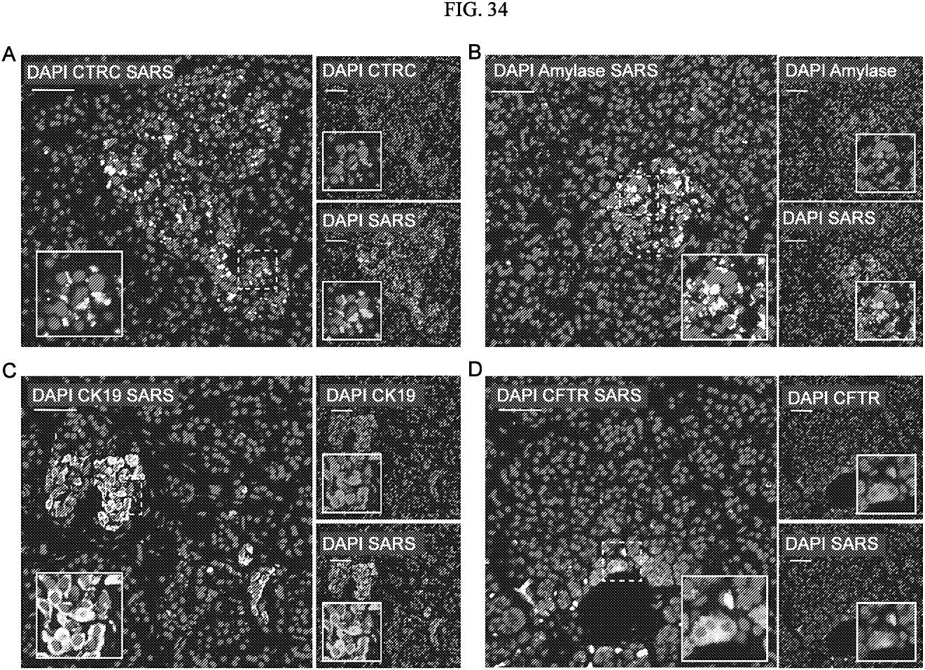

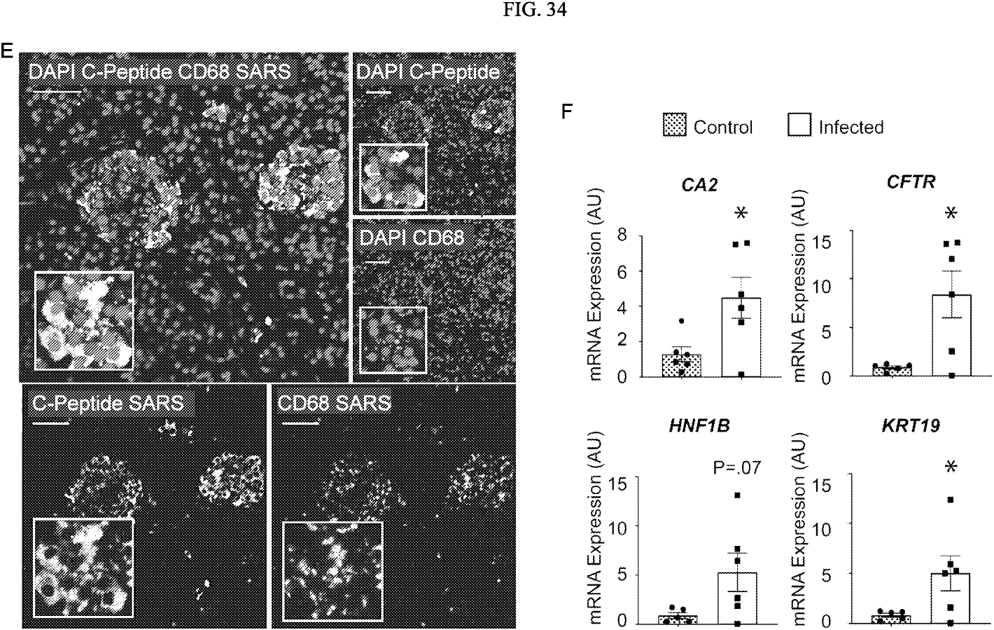

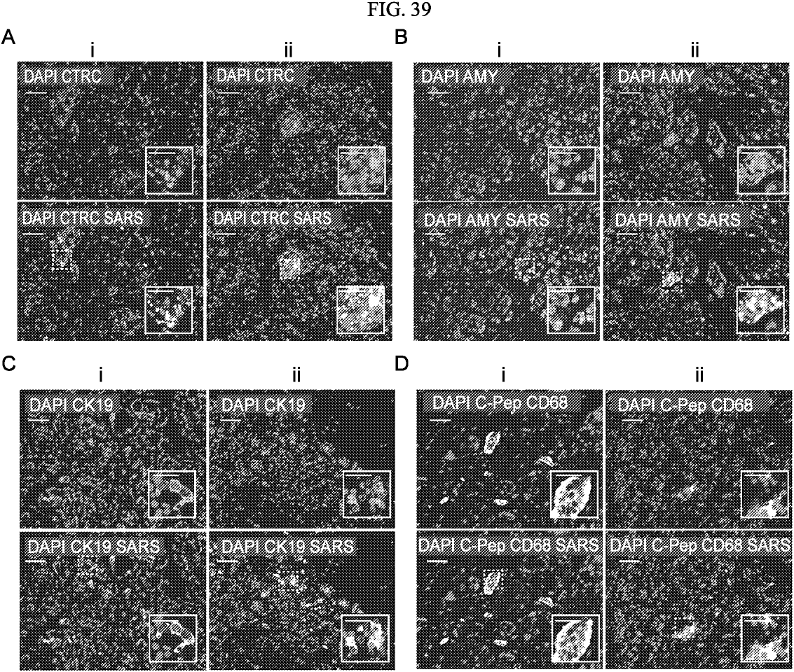

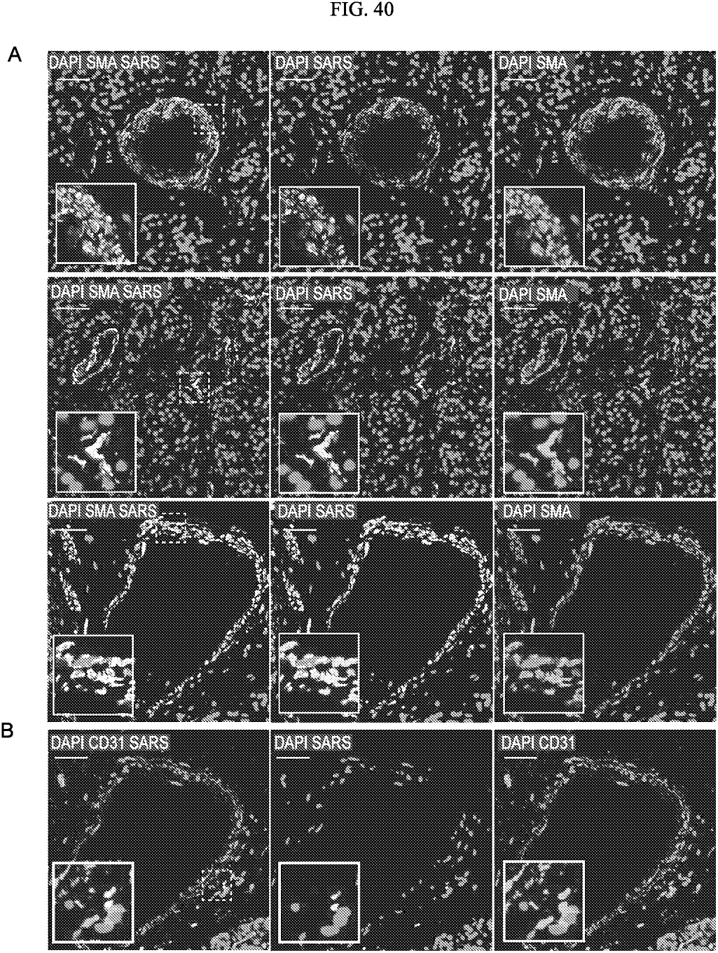

Post-mortem human pancreatic samples were obtained from individuals who succumbed from complications related to COVID-19 infection (COVID-19 patients) or from those who were not infected by SARS-CoV-2 and were deceased due to complications unrelated to COVID-19 (Control patients). Clinical metadata of the patients whose samples were utilized in this study. Samples were processed for immunohistochemistry of SARS-CoV-2 and pancreatic markers and processed for analysis of changes in gene expression. As shown in FIGS. 34 A-Eand FIGS. 39 and 40, SARS-CoV-2 was detected in pancreata of COVID-19 patients, demonstrating the susceptibility of the human pancreas to the virus. To better understand which cell types were infected by SARS-CoV-2 in the pancreas, cells were stained for specific endocrine and exocrine markers, such as Amylase and Chymotrypsin (CTRC) for acinar cells, Cytokeratin 19 (CK19) and Cystic fibrosis transmembrane conductance regulator (CFTR) for ductal cells, and C-peptide for endocrine β-cells. Interestingly, many of the pancreatic cell types tested here co-localized with SARS-CoV-2 in some or all patients. The virus was present in the majority of the CTRC⁺ and some clusters of Amylase⁺ cells (FIGS. 34A-B). Similar staining patterns of CTRC and Amylase with SARS-CoV-2 in two other patients can be found in FIGS. 39A-B. As for the ductal population, SARS-CoV-2 was also detected in many CK19⁺ cells (FIG. 34C, FIG. 39C) and few cells expressing CFTR (FIG. 34D). SARS-CoV-2 staining is frequently concentrated in C-Peptide⁺ islet clusters (FIG. 34E, FIG. 39D) along with some peripheral CD68⁺ macrophages that are present within and surrounding the islets (FIG. 34E). Other cell types present in the pancreas were also investigated, such as vascular endothelial and smooth muscle cells, and were stained with CD31 and Smooth Muscle Actin (SMA), respectively (FIG. 40). CD31 cells seem less prone to SARS-CoV-2 infection, while SMA⁺ cells showed evidence of overlapping with SARS-CoV-2 staining in post-mortem tissues.

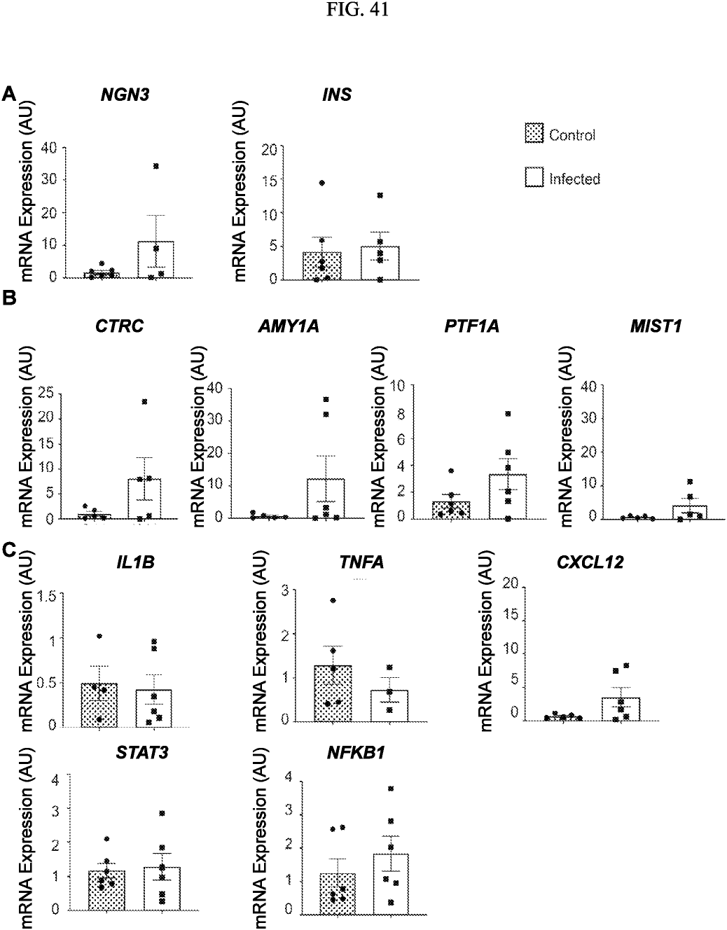

To examine whether the infected pancreatic tissues from COVID-19 patients may have differential expression of specific genes, snap-frozen post-mortem tissues from COVID-19 patients and Control subjects were assessed changes in expression of pancreas-specific genes. Interestingly, mRNA expression of ductal markers was increased in the COVID-19 samples, such as KRT19, CFTR, CA2, and HNF1B as seen in FIG. 34F. Other pancreatic and inflammatory markers were also tested and although showed trends of perturbations, they were not statistically different between the groups likely due to the higher variability in a subset of COVID-19 patients (FIG. 41). These results from COVID-19 and Control human tissues corroborate our novel results in iPan cell cultures, where we show that both pancreatic endocrine and exocrine cells can be infected by SARS-CoV-2, and the infection causes perturbations in pancreas-specific genes and the pancreatic cellular machinery.

### Additional Representative Embodiments

A method, comprising: providing one or more populations of cell types in a fluidic device; infecting the one or more populations of cell types with a virus; adding one or more candidate agents to the infected one or more populations of cell types; measuring one or more phenotypes of interest in the infected one or more populations of cell types after adding the one or more candidate agents; and selecting one or more candidate agents based on measurements of the one or more phenotypes of interest in the one or more populations of cell types.

The method of the preceding paragraph, wherein the one or more phenotypes of interest is wherein phenotype of interest is viral infection, replication, or both.

The method of a preceding paragraph, wherein the virus is a coronavirus.

The method of a preceding paragraph, wherein the coronavirus is SARS-Cov-1 or SARS-Cov-2.

The method of a preceding paragraph, wherein the one or more phenotypes of interest is modulating of IFN, TLR, NFKB, Th1 and Th2 pathways.

The method of a preceding paragraph, wherein each of the one or more populations of cell types is organized as an organoid.

The method of a preceding paragraph, comprising lung, heart, or organoid.

The method of a preceding paragraph, wherein the one or more populations of cell types in a fluidic device is an air liquid interface.

The method of a preceding paragraph, wherein the one or more populations of cell types in a fluidic device is an organ chip.

The method of a preceding paragraph, wherein the one or more populations of cell types in a fluidic device is an organ chip.

The method of a preceding paragraph, wherein the one or more candidate agents is a nucleotide analog.

The method of a preceding paragraph, wherein the one or more candidate agents is an anti-inflammatory molecule.

The method of a preceding paragraph, wherein the one or more candidate agents is selected from the group consisting of: interferon beta, ribavirin, chloroquine, azithromycin, favipiravir, lopinavir/ritonavir, and remdesivir.

The method of a preceding paragraph, wherein the one or more populations of cell types are selected from the group consisting of: lung, heart, and endothelial cells.

The method of a preceding paragraph, wherein the lung cells are proximal air way cells.

The method of a preceding paragraph, wherein the lung cells are distal alveolar organoids.

The method of a preceding paragraph, wherein the heart cells are cardiac myocytes.

The method of a preceding paragraph, wherein the one or more population of cell types are derived from induced pluripotent stem cells (iPSCs).

An apparatus comprising one or more populations of cell types in a fluidic device comprising an apical chamber a basement chamber.

An apparatus comprising one or more populations of cell types in a fluidic device comprising an organ chip.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the openended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A system for infection modeling or test agent screening, comprising:
a population of cells comprising cells selected from the group consisting of progenitor cells derived from induced pluripotent stem cells (iPSCs), cells differentiated from iPSCs, an organoid comprising the cells differentiated from iPSCs, or primary cells; and
a fluidic device, a cell culture plate, or a multi-well culture plate;
wherein an infectious agent and the population of cells, or the infectious agent, a test agent, and the population of cells, are in contact in the fluidic device, the cell culture plate, or the multi-well culture plate.

2. The system of claim 1, wherein:
the fluidic device is an air-liquid interface culture or a Transwell system comprising the population of cells;
the fluidic device is a microfluidic device comprising the population of cells; and/or
the fluidic device is an organ chip.

3. (canceled)

4. (canceled)

5. The system of claim 1, wherein:
the progenitor cells comprise progenitor heart cells or progenitor endothelial cells;
the primary cells comprise heart cells or endothelial cells; and/or
the heart cells are cardiac myocytes.

6. (canceled)

7. (canceled)

8. The system of claim 1, wherein:
the progenitor cells comprise progenitor lung cells or progenitor pancreatic cells;
the primary cells comprise lung cells or pancreatic cells;
the lung cells are epithelial cells; and/or
the epithelial cells are proximal airway cells, or distal alveolar cells.

9. (canceled)

10. (canceled)

11. (canceled)

12. The system of claim 1, wherein:
the infectious agent is a virus, a bacterium, a fungus, or a parasite;
the virus is a coronavirus; and/or
the virus is SARS-CoV-1, MERS, or SARS CoV-2.

13. (canceled)

14. (canceled)

15. The system of claim 1, wherein:
the test agent is an anti-viral agent; or
the test agent is a nucleotide analog, an anti-inflammatory agent, interferon beta, ribavirin, chloroquine, azithromycin, favipiravir, lopinavir/ritonavir or remdesivir.

16. (canceled)

17. A method selecting an agent of interest, comprising:
contacting a test agent with a population of cells comprising cells selected from the group consisting of progenitor cells derived from induced pluripotent stem cells (iPSCs), cells differentiated from iPSCs, an organoid comprising the cells differentiated from iPSCs, or primary cells, wherein the test agent and the population of cells are in contact in a fluidic device, a cell culture plate, or a multi-well culture plate;
infecting the population of cells with an infectious agent before, simultaneously or after contacting the test agent with the population of cells;
measuring a parameter in the population of cells; and
selecting the test agent as the agent of interest based on the measured parameter in the population of cells.

18. A method of studying an infectious agent, comprising:
contacting an infectious agent with a population of cells comprising cells selected from the group consisting of progenitor cells derived from induced pluripotent stem cells (iPSCs), cells differentiated from iPSCs, an organoid comprising the cells differentiated from iPSCs, or primary cells, wherein the infectious agent and the population of cells are in contact in a fluidic device, a cell culture plate, or a multi-well culture plate; and
measuring a parameter in the population of cells.

19. The method of claim 17, wherein:
the parameter comprises a phenotype of interest, an expression level of a gene, or an expression level of a protein of interest, or combination thereof;
the fluidic device is an air-liquid interface culture or a Transwell system comprising the population of cells; and/or
the fluidic device is a microfluidic device comprising the population of cells.

20. (canceled)

21. (canceled)

22. The method of claim 17, wherein:
the progenitor cells comprise progenitor lung cells, progenitor heart cells, progenitor endothelial cells, or pancreatic progenitor cells;
the primary cells comprise lung cells, heart cells, endothelial cells, or pancreatic cells;
the lung cells are epithelial cells;
the epithelial cells are proximal airway cells, or distal alveolar cells; and/or
the heart cells are cardiac myocytes.

23. (canceled)

24. (canceled)

25. (canceled)

26. (canceled)

27. (canceled)

28. (canceled)

29. (canceled)

30. The method of claim 17, wherein the test agent is an anti-viral agent.

31. The method of claim 17, wherein the test agent is a nucleotide analog, an anti-inflammatory agent, interferon beta, ribavirin, chloroquine, azithromycin, favipiravir, lopinavir/ritonavir or remdesivir.

32. The method of claim 17, wherein the parameter comprises a phenotype of interest, expression level of a gene of interest, expression level of a protein of interest, or combinations thereof in the population of cells.

33. A method of selecting an agent of interest, comprising:
contacting a test agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof;
measuring a phenotype of interest or expression level of a gene or protein of interest in the population of cells; and
selecting the test agent as the agent of interest based on the measurement of the phenotype of interest or expression level of the gene or protein of interest.

34. The method of claim 33, further comprising:
infecting the population of cells with an infectious agent before, simultaneously or after contacting the test agent with the population of cells.

35. A method of studying an infectious agent, comprising:
contacting an infectious agent with a population of cells comprising cells selected from the group consisting of pancreatic progenitor cells derived from induced pluripotent stem cells (iPSCs), pancreatic ductal cells derived from iPSCs, pancreatic ductal cells derived from pancreatic progenitor cells, pancreatic acinar cells derived from iPSCs, pancreatic acinar cells derived from pancreatic progenitor cells, an organoid comprising pancreatic acinar cells derived from iPSCs, an organoid comprising pancreatic acinar cells derived from pancreatic progenitor cells, an organoid comprising pancreatic ductal cells derived from iPSCs, an organoid comprising pancreatic ductal cells derived from pancreatic progenitor cells, and combinations thereof; and
measuring a phenotype of interest or expression level of a gene or protein of interest in the population of cells.

36. The method of claim 33, wherein the test agent and the population of cells are in contact in a fluidic device, or a cell culture plate, or a multi-well culture plate.

37. The method of claim 36, wherein the fluidic device, or the cell culture plate, or the multi-well culture plate is a Transwell system.

38. The method of claim 36, wherein the fluidic device is a microfluidic device.

39. The method of claim 33, wherein:
the population of cells selected are from the group consisting of pancreatic progenitor cells derived from induced pluripotent stem cells (iPSCs), pancreatic ductal cells derived from iPSCs, pancreatic acinar cells derived from iPSCs, an organoid comprising derived from iPSCs, an organoid comprising pancreatic acinar cells derived from iPSCs, an organoid comprising pancreatic ductal cells derived from iPSCs, and combinations thereof; or
the population of cells selected are from the group consisting of pancreatic ductal cells derived from pancreatic progenitor cells, pancreatic acinar cells derived from pancreatic progenitor cells, an organoid comprising pancreatic acinar cells derived from pancreatic progenitor cells, an organoid comprising pancreatic ductal cells derived from pancreatic progenitor cells, and combinations thereof.

40. (canceled)

41. The method of claim 34, wherein;
the infectious agent is a virus, bacterium, parasite, or fungus;
the infectious agent is a coronavirus; and/or
the infectious agent is SARS-CoV-2.

42. (canceled)

43. (canceled)

44. The method of claim 33, wherein the test agent is selected from the group consisting of interferon beta, ribavirin, chloroquine, azithromycin, favipiravir, lopinavir/ritonavir, and remdesivir.
